SEQUENCE LISTING (114 sequences)

SEQ ID NO: 1 - protein, 133 aa, Homo sapiens [sequence omitted]

SEQ ID NO: 2 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSII

SEQ ID NO: 3 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIL

SEQ ID NO: 4 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADRTATIVEFLNRWITFCQSII

SEQ ID NO: 5 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWYTFCQSII

SEQ ID NO: 6 - protein, 129 aa, synthetic construct
APTSSSTKKTQLQLEHLLLDLQMILNGPNNYKNPKLTRMLTFKFYMPKKATELKHLQCLEEELKPLEEVLNLAQSKNFHLRPRDLISNINVIVLELKGSETTFMCEYADETATIVEFLNRWITFCQSII

SEQ ID NO: 7 - protein, 129 aa, synthetic construct
APTSSSTKKTQLQLEHLLLDLQMILNGINNYDNPKLTRMLTFKFYMPKKATELKHLQCLEEELKPLEEVLNLAQSKNFHLRPRDLISNINVIVLELKGSETTFMCEYADETATIVEFLNRWITFCQSII

SEQ ID NO: 8 - protein, 129 aa, synthetic construct
APTSSSTKKTQLQLEHLLLDLQMILNGINNYKNPKLTRMLTFKFYMPKKATGLKHLQCLEEELKPLEEVLNLAQSKNFHLRPRDLISNINVIVLELKGSETTFMCEYADETATIVEFLNRWITFCQSII

SEQ ID NO: 9 - protein, 129 aa, synthetic construct
APTSSSTKKTQLQLEHLLLDLQMILNGINNYKNPKLTRMLTFKFYMPKKATELKHLQCLEEELKPLEEVLNLAQSRNFHLRPRDLISNINVIVLELKGSETTFMCEYADETATIVEFLNRWITFCQSII

SEQ ID NO: 10 - protein, 129 aa, synthetic construct
APTSSSTKKTQLQLEHLLLDLQMILNGINNYKNPKLTRMLTFKFYMPKKATELKHLQCLEEELKPLEEVLNLAQSKNGHLRPRDLISNINVIVLELKGSETTFMCEYADETATIVEFLNRWITFCQSII

SEQ ID NO: 11 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRYRDLISNINVIVLELKGSETTFMCEYADETATIVEFLNRWITFCQSII

SEQ ID NO: 12 - protein, 129 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADRTATIVEFLNRWITFCQSIL

SEQ ID NO: 13 - protein, 129 aa, synthetic construct
APTSSSTKKTQLQLEHLLLDLQMILNGINNYDNPKLTRMLTFKFYMPKKATELKHLQCLEEELKPLEEVLNLAQSKNFHLRPRDLISNINVIVLELKGSETTFMCEYADETATIVEFLNRWITFCQSIL

SEQ ID NO: 14 - protein, 133 aa, synthetic construct
APTSSSTKKTQLQLEHLLLDLQMILNGINNYKNPKLTRMLTVKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 15 - protein, 361 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 361 aa, synthetic construct
APTSSSTKKTQLQLEHLLLDLQMILNGINNYKNPKLTRMLTVKFAMPKKATELKHLQCLEEELKPLEEVLNGAQSKNFHLRPRDLISNINVIVLELKGSETTFMCEYADETATIVEFLNRWITFCQSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 17 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADRTATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 357 aa, synthetic construct; identical to SEQ ID NO: 19

SEQ ID NO: 21 - protein, 357 aa, synthetic construct
APTSSSTKKTQLQLEHLLLDLQMILNGPNNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 357 aa, synthetic construct
APTSSSTKKTQLQLEHLLLDLQMILNGINNYDNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 357 aa, synthetic construct
APTSSSTKKTQLQLEHLLLDLQMILNGINNYKNPKLTRMLTFKFYMPKKATGLKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 24 - protein, 357 aa, synthetic construct
APTSSSTKKTQLQLEHLLLDLQMILNGINNYKNPKLTRMLTFKFYMPKKATELKHLQCLEEELKPLEEVLNLAQSRNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - protein, 357 aa, synthetic construct
APTSSSTKKTQLQLEHLLLDLQMILNGINNYKNPKLTRMLTFKFYMPKKATELKHLQCLEEELKPLEEVLNLAQSKNGHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRYRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 357 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWYTFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 357 aa, synthetic construct
APTSSSTKKTQLQLEHLLLDLQMILNGINNYDNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADRTATIVEFLNRWYTFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 358 aa, synthetic construct
APTSSSTKKTQLQLEHLLLDLQMILNGINNYDNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 446 aa, synthetic construct
EIQLQQSGPELVKPGASVKVSCKASGYSFTDYNMYWVKQSHGESLEWIGYIDPYNGGTRYNQKFKGKATLTVDKSSSTAFMHLNSLTSEDSAVYYCARNGHWDGAWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 34 - protein, 214 aa, synthetic construct
DIQMNQSPSSLSASLGDTITITCHASQNINVWLSWYQQKPGNIPKLLIYKASNLHTGVPSRFSGSRSGTGFTLTISSLQPEDIATYYCHQGQSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 198 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCHHHHHH

SEQ ID NO: 36 - protein, 230 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDLEGGGGSGGGGHHHHHH

SEQ ID NO: 37 - protein, 249 aa, synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENLEGGGGSGGGGHHHHHH

SEQ ID NO: 38 - protein, 370 aa, synthetic construct
APTSSSTSSSTAEAQQQQQHLEQLRMDLEELLSRMENYRNLKLPRMLTFKFYLPKQATELKDLOCLEDELGPLRHVLDLTOSKSFQLEDAENFISNIRVTVVKLKGSDNTFECOFDDESATVVDFLRRWIAFCHSIISTSPOESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 39 - protein, 361 aa, synthetic construct
APTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCKSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 40 - protein, 594 aa, synthetic construct
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 41 - protein, 442 aa, synthetic construct
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 42 - protein, 219 aa, synthetic construct
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 362 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 45 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 576 aa, synthetic construct
EVQLVESGGGLVKPGGSLELSCAASGFTFSSYWMSWVRQAPEKGLEWVAAISPSGGSTYYADSVKGRFTISRDNAKNTLFLQMTSLRSEDTAMYYCAKESWGAYYDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGAWVNVISDLKKIEDLIQSMHIDATLYTESNVHPSCKVTAMKCFLLGLQRISLESGDASIHDTVENLIILANNSLSSNGNVTESGCKECEELEEKNIKEFLQSFVHIVQMFINTS

SEQ ID NO: 47 - protein, 448 aa, synthetic construct
EVQLVESGGGLVKPGGSLELSCAASGFTFSSYWMSWVRQAPEKGLEWVAAISPSGGSTYYADSVKGRFTISRDNAKNTLFLQMTSLRSEDTAMYYCAKESWGAYYDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
DIVMTQSPSSLSVSAGDKVTMSCRASQGISSWLAWYQQKPWQPPKLLIYKASTLESGVPDRFTGSGSGTDFTLTISSVQAEDLAVYYCQQSYSTPWTFGGGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 447 aa, synthetic construct
QVQLVQSGAEVKKPGSSVKVSCKASGYTFTSYWINWVRQAPGQGLEWMGNIYPGSSITNYAQKFQGRVTITADESTSTAYMELSSLRSEDTAVYYCARLTTGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 575 aa, synthetic construct
QVQLVQSGAEVKKPGSSVKVSCKASGYTFTSYWINWVRQAPGQGLEWMGNIYPGSSITNYAQKFQGRVTITADESTSTAYMELSSLRSEDTAVYYCARLTTGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGGWVNVISDLKKIEDLIQSMHIDATLYTESNVHPSCKVTAMKCFLLGLQRISLESGDASIHDTVQNLIILANNSLSSNGNVTESGCKECEELEEKNIKEFLQSFVHIVQMFINTS

SEQ ID NO: 51 - protein, 220 aa, synthetic construct
DIQMTQSPSSLSASVGDRVTITCKSSQSLWDSGNQKNFLTWYQQKPGKAPKLLIYWTSYRESGVPSRFSGSGSGTDFTLTISSLQPEDFATYYCQNDYFYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 358 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 358 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 358 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIIESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 358 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 442 aa, synthetic construct; identical to SEQ ID NO: 41

SEQ ID NO: 58 - protein, 219 aa, synthetic construct; identical to SEQ ID NO: 42

SEQ ID NO: 59 - protein, 358 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADRTATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 362 aa, synthetic construct
APTSSSTKKTQLQLEHLRLDLEMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCKSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 362 aa, synthetic construct
APTSSSTKKTQLQLEHLLHDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFIESIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 362 aa, synthetic construct
APASSSTKKTQLQLEHLLLNLQMILNGINNYKNPKLTRMLTIKFYMPKKATELKHLQCLEEELKPLEEVLKLAQSKNFHLRPRDLISNINVIVLELKGSETTFMCEYADETATIVEFLNRWITFSQSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 358 aa, synthetic construct
APTSSSTKKTQLDLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRKLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLIRNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISKINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINEIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVRVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVDVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVDELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLRLKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLRRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWIKFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 471 aa, synthetic construct
QVQLVESGGGVVQPGGSLRLSCAASDSIDSLVNMGWYRQAPGKQRELVALIATYITHYADFVKGRFTISRDNSKNTLYLQMNSLRAEDTAVYYCYARNIIVDYWGQGTLVTVSSGGGGSGGGGSGGGGSQVQLVESGGGVVQPGGSLRLSCAASDSIDSLVNMGWYRQAPGKQRELVALIATYITHYADFVKGRFTISRDNSKNTLYLQMNSLRAEDTAVYYCYARNIIVDYWGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 76 - protein, 362 aa, synthetic construct
APTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCKSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 362 aa, synthetic construct
APTSSSTKKTQLQLEHLLHDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIESIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 362 aa, synthetic construct
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 129 aa, synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSII

SEQ ID NO: 80 - protein, 129 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSIL

SEQ ID NO: 81 - protein, 129 aa, synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADRTATIVEFLNRWITFCQSIL

SEQ ID NO: 82 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWITFCQSIL

SEQ ID NO: 83 - protein, 133 aa, synthetic construct
APTSSSTKKTQLQLEHLRLDLEMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 84 - protein, 133 aa, synthetic construct
APTSSSTKKTQLQLEHLLHDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFIESIISTLT

SEQ ID NO: 85 - protein, 133 aa, synthetic construct
APASSSTKKTQLQLEHLLLNLQMILNGINNYKNPKLTRMLTIKFYMPKKATELKHLQCLEEELKPLEEVLKLAQSKNFHLRPRDLISNINVIVLELKGSETTFMCEYADETATIVEFLNRWITFSQSIISTLT

SEQ ID NO: 86 - protein, 129 aa, synthetic construct
APTSSSTKKTQLDLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWITFCQSIL

SEQ ID NO: 87 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRKLISNINVIVLELKGSETTFMCEYADETATIVEFLNRWITFCQSIL

SEQ ID NO: 88 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLIRNINVIVLELKGSETTFMCEYADETATIVEFLNRWITFCQSIL

SEQ ID NO: 89 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISKINVIVLELKGSETTFMCEYADETATIVEFLNRWITFCQSIL

SEQ ID NO: 90 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINEIVLELKGSETTFMCEYADETATIVEFLNRWITFCQSIL

SEQ ID NO: 91 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVRVLELKGSETTFMCEYADETATIVEFLNRWITFCQSIL

SEQ ID NO: 92 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVDVLELKGSETTFMCEYADETATIVEFLNRWITFCQSIL

SEQ ID NO: 93 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVDELKGSETTFMCEYADETATIVEFLNRWITFCQSIL

SEQ ID NO: 94 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLRLKGSETTFMCEYADETATIVEFLNRWITFCQSIL

SEQ ID NO: 95 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLRRWITFCQSIL

SEQ ID NO: 96 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWIKFCQSIL

SEQ ID NO: 97 - protein, 129 aa, synthetic construct
APTSSSTKKTQLRLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWITFCQSIL

SEQ ID NO: 98 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRTLISNINVIVLELKGSETTFMCEYADETATIVEFLNRWITFCQSIL

SEQ ID NO: 99 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLIININVIVLELKGSETTFMCEYADETATIVEFLNRWITFCQSIL

SEQ ID NO: 100 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINSIVLELKGSETTFMCEYADETATIVEFLNRWITFCQSIL

SEQ ID NO: 101 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLYLKGSETTFMCEYADETATIVEFLNRWITFCQSIL

SEQ ID NO: 102 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLQRWITFCQSIL

SEQ ID NO: 103 - protein, 129 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWIVFCQSIL

SEQ ID NO: 104 - protein, 358 aa, synthetic construct
APTSSSTKKTQLRLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 357 aa, synthetic construct
APTSSSTKKTQLQLEHLLLDLQMILNGINNYDNPKLTRMLTIKFYMPKKATELKHLQCLEEELKPLEEVLNLAQSKNFHLRPRTLISNINVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 106 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLIININVIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINSIVLELKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLYLKGSETTFMCEYADETATIVEFLNRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLQRWITFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 358 aa, synthetic construct
APTSSSTKKTQLQLEHLLLDLQMILNGINNYDNPKLTRMLTIKFYMPKKATELKHLQCLEEELKPLEEVLNLAQSKNFHLRPRDLISNINVIVLELKGSETTFMCEYADETATIVEFLNRWIVFCQSILESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 129 aa, synthetic construct
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIL

SEQ ID NO: 112 - protein, 133 aa, synthetic construct
APTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 113 - protein, 133 aa, synthetic construct
APTSSSTKKTQLQLEHLLHDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIESIISTLT

SEQ ID NO: 114 - protein, 133 aa, synthetic construct
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLT

(19) United States
(12) Patent Application Publication
(10) Pub. No.: US 2026/0232769 A1
(43) Pub. Date: Aug. 13, 2026

(54) IL-2 VARIANTS WITH IMPROVED STABILITY AND COMPOSITIONS THEREOF

(71) Applicant: WUXI BIOLOGICS IRELAND LIMITED, Co Louth (IE)

(72) Inventors: Lei Wu, Shanghai (CN); Mengmeng Sun, Shanghai (CN); Shuang Wang, Shanghai (CN); Meng Wu, Shanghai (CN); Jianqing Xu, Shanghai (CN); Jijie Gu, Shanghai (CN)

(21) Appl. No.: 19/469,869

(22) PCT Filed: Mar. 29, 2024
(86) PCT No.: PCT/CN2024/084866

§ 371 Date: Sep. 26, 2025

(30) Foreign Application Priority Data
Mar. 29, 2023 (WO) PCT/CN2023/084766

(51) Int. Cl. A61K 38/20; A61P 35/00; C07K 14/55

(52) U.S. Cl. CPC A61K 38/2013; A61P 35/00; C07K 14/55

(57) ABSTRACT

Provided are IL-2 variants, polypeptide complexes comprising the IL-2 variants and compositions comprising the polypeptide complexes, the methods of producing the same and the uses thereof. The IL-2 variants and polypeptide complexes of the disclosure can be used as a potent agent for the treatment of cancers, autoimmune and inflammatory diseases.

### CROSS-REFERENCES

This application is a national stage application under 35 U.S.C. § 371 of International Patent Application No. PCT/CN2024/084866, filed Mar. 29, 2024, which claims priority to International Patent Application No. PCT/CN2023/084766, filed on Mar. 29, 2023, the contents of each of which are incorporated by reference herein in their entireties.

### SEQUENCE LISTING

The instant application contains a sequence listing which has been submitted electronically in XML format and is hereby incorporated by reference in its entirety. Said XML copy, created on Sep. 26, 2025, is named 000516-0002-301-SL.xml and is 130,082 bytes in size.

### FIELD

This application generally relates to IL-2 variants, fusion proteins and polypeptide complexes comprising the IL-2 variant, a method for preparing the same, and the uses thereof.

### BACKGROUND

Interleukin-2 (IL-2) is a four α-helical bundle cytokine identified as a growth factor of T cells, involving in the growth and proliferation of many immune cells [Morgan D A, Ruscetti F W, and Gallo R. Selective in vitro growth of T lymphocytes from normal human bone marrows. Science 1976; 193:1007-1008]. IL-2 is mainly produced by activated T cells. The biological activity of IL-2 is mediated through three transmembrane receptor subunits: IL-2Rα (CD25), IL-2Rβ (CD122) and common γ chain (CD132). NK cells and steady state T cells express IL-2Rβ and γ, and only regulatory T cells (Treg) and activated T cells express IL-2Rα in addition to IL-2Rβ and γ. IL-2Rα serves to facilitate the delivery of IL-2 to the other 2 subunits, and IL-2Rβ/γ transduce activating signals through STAT5-related pathway. The IL-2Rα/β/γ trimer on Treg and activated T cells works as the high affinity receptor (K_{D}~10⁻¹¹M) of IL-2, and IL-2Rβ/γ (on NK cells and steady state T cells) is the intermediate affinity receptor (K_{D}~10⁻⁹ M) [Takeshita T, Asao H, Ohtani K, et al. Cloning of the gamma chain of the human IL-2 receptor. Science 1992; 257:379-382]. The high affinity receptor is critical to capture IL-2 in lower concentration and makes Treg and activated T cells more sensitive to IL-2 to support their respective functions. Especially for cancer immunotherapy, IL-2Rα engagement with IL-2 plays an important role to program stem-like T cells [Hashimoto M, Araki K, Cardenas M A, et al. PD-1 combination therapy with IL-2 modifies CD8+ T cell exhaustion program. Nature. 2022; 610:173-181].

IL-2 and its variants have been tested in various clinical trials. IL-2 has shown promising efficacy in cancer immunotherapy, but its toxicity profile has limited its efficacy and use. Recombinant IL-2 showed short half-life and severe side effects such as vascular leak syndrome (VLS). IL-2 variants have been developed to mitigate such problems, however, they have encountered issues including mitigated efficacy or elevated immunogenicity. Fusion proteins containing IL-2 or its variants, such as IL-2-Fc fusion proteins, can have fragile structure, low stability, and bad pharmacokinetic profile. These features have severely limited its clinical prospect.

Cancer immunotherapy using immuno-checkpoint inhibitors such as anti-PD (L) 1 antibodies and other immune modulatory drugs have achieved breakthrough in recent years. However, a significant proportion of cancer patients still remain resistant or refractory to existing cancer immunotherapies. IL-2, as a T and NK cells modulatory cytokine, has the potential to augment responses induced by existing therapies, like therapeutic antibodies to checkpoints (PD1, PD-L1, CTLA4, LAG-3, etc.) or tumor association antigens (Rituximab, Trustuzumab, Daratumumab, etc).

Treg cells play a critical role to maintain immune homeostasis and self-tolerance, and is crucial in controlling the development of allergies and autoimmune diseases. IL-2 is able to induce the proliferation of Treg cells by signaling through high affinity IL-2 receptor. Therefore, expanding Treg population with IL-2 therapy has the potential to balance and limit pathogenic T cells in inflammatory diseases. At present, IL-2 therapy is being tested in clinical trials for inflammatory diseases including graft-versus-host disease, type 1 diabetes and systemic lupus erythematosus [Ye C, Brand D, and Zheng S. G. Targeting IL-2: an unexpected effect in treating immunological diseases. Sig Transduct Target Ther 2018]. Nevertheless, existing IL-2 molecules are still burdened by fast clearance and potential toxicity.

Therapeutic IL-2, therefore, still need further exploration and optimization to generate ideal drug molecules with powerful efficacy, in vivo stability, good pharmacokinetics, and limited toxicity. IL-2 based therapies may meet the huge medical needs in immuno-oncology and autoimmune disease therapeutic areas.

The present disclosure provides potency reduced, stabilized IL-2 variants and IL-2 fusion proteins thereof with improved pharmacokinetics, which may serve as a novel immunotherapy agent with improved therapeutic efficacy.

### SUMMARY

These and other objectives are provided for by the present disclosure which, in a broad sense, is directed to compounds, methods, compositions and articles of manufacture that provide proteins with improved efficacy. The benefits provided by the present disclosure are broadly applicable in the field of therapeutics and diagnostics and may be used in conjunction with antibodies that react with a variety of targets.

The present invention is directed to human IL-2 variants, fusion proteins and polypeptide complexes comprising the IL-2 variants. The IL-2 is herein engineered with a unique approach to produce variants—with better in vivo stability and pharmacokinetics, attenuated affinity to IL-2Rβ/γc, to IL-2Rα, or to both and the combined IL-2Rα/β/γc complex. Through stabilization, the IL-2 variant gets better thermal-stability, better serum stability, extended in vivo half-life, slower in vivo clearance, but with limited toxicity. Though attenuated, the IL-2 variants and the polypeptide complexes comprising the IL-2 variants still retain binding capacity to IL-2Rα, to IL-2Rβ/γc, and to the combined IL-2Rα/β/γc complex. The IL-2 variants have higher affinity to IL-2Rα/β/γc complex than to IL-2Rα or IL-2Rβ/γ and remain stable in vivo.

The IL-2 variants and the polypeptide complexes comprising the IL-2 variants showed different potency and toxicity. The IL-2 variants and the polypeptide complexes comprising the IL-2 variants hence may serve as a novel immunotherapy agent for cancer and autoimmune disease, as a standalone therapy or in combination with other treatment options. In addition, the IL-2 variants and the polypeptide complexes comprising the IL-2 variants may serve as an immunomodulatory agent for autoimmunity and other inflammatory diseases.

In some aspects, the present disclosure provides an IL-2 variant, wherein the IL-2 variant has a modified (more specifically, reduced) binding affinity to at least one of IL-2Rα, IL-2Rβ, common γ chain and IL-2Rα/β/γc complex, or improved in vivo stability and pharmacokinetics, and has an amino acid sequence comprising one or more mutation(s) selected from a C-terminal truncation, substitutions at position 32, 129, 13, 18, 19, 20, 22, 28, 38, 42, 52, 71, 76, 78, 82, 84, 87, 88, 89, 91, 92, 94, 95, 110, 119, 122, 123, 125 and 126 and any combinations thereof, compared to the amino acid sequence as set forth in SEQ ID NO: 1.

In some aspects, the present disclosure provides a composition comprising a polypeptide complex or a nucleic acid molecule(s) encoding the polypeptide complex as active ingredient, and an excipient,
wherein the polypeptide complex comprises an interleukin-2 (IL-2) variant domain, a first dimerization domain and a second dimerization domain, the IL-2 variant domain comprises an amino acid sequence with a truncation of 1-20 amino acids from the C terminal and a substitution at one or more positions selected from positions 32, 129, 13, 18, 19, 20, 22, 28, 38, 42, 52, 71, 76, 78, 82, 84, 87, 88, 89, 91, 92, 94, 95, 110, 119, 122, 123, 125 and 126 in relation to SEQ ID NO: 1, and the first dimerization domain and the second dimerization domain associates together to form a dimer,
wherein the polypeptide complex has a reduced binding affinity to at least one of IL-2Rα, IL-2Rβ/γc and IL-2Rα/β/γc complex compared to an otherwise identical polypeptide complex comprising wild-type IL-2 instead of the IL-2 variant.

In some embodiments, the polypeptide complex or the nucleic acid molecule encoding the polypeptide complex is less than 90%, less than 80%, less than 70%, less than 60% or less than 50% by weight of the composition.

In some embodiments, the IL-2 variant comprises a truncation of 20, 15, 10, 9, 8, 7, 6, 5, 4, 3, 2 or 1 amino acid(s) from the C terminal in relation to SEQ ID NO: 1.

In some embodiments, the IL-2 variant comprises a substitution at one or more positions selected from positions 32, 28, 52, 76, 78 and 82, in combination with a substitution at one or more positions selected from positions 129, 110 and 122, in relation to SEQ ID NO: 1.

In some embodiments, the IL-2 variant comprises a substitution at one or more positions selected from positions 13, 84, 87, 88, 91, 92, 94, 119 and 123, in combination with a substitution at one or more positions selected from positions 32, 42 and 129, in relation to SEQ ID NO: 1.

In some embodiments, the IL-2 variant comprises a substitution at one or more positions selected from positions 3, 18, 19, 20, 22, 71, 125 and 126, in combination with a substitution at one or more positions selected from positions 32, 42 and 129, in relation to SEQ ID NO: 1.

In some embodiments, the IL-2 variant comprises a substitution at position 32 selected from K32D, K32A, K32E, K32G, K32V, K32F, K32M, K32W, K32P, K32Q, K32I, K32S, K32T, K32N, K32R, K32H, K32L and K32Y, preferably the K residue is substituted by an amino acid residue with opposite charge. Additionally or alternatively, the IL-2 variant comprises a substitution at position 129 selected from I129L, I129A, I129E, I129G, I129V, I129F, I129M, I129W, I129P, I129Q, I129K, I129S, I129T, I129N, I129D, I129R, I129H and I129Y.

In some embodiments, the IL-2 variant comprises a substitution selected from any of the following:
(a) a substitution at position 110 selected from E110R, E110A, E110K, E110G, E110V, E110F, E110M, E110W, E110P, E110Q, E110I, E110S, E110T, E110N, E110D, E110H, E110L and E110Y;
(b) a substitution at position 52 selected from E52G, E52A, E52K, E52V, E52F, E52M, E52W, E52P, E52Q, E52I, E52S, E52T, E52N, E52D, E52R, E52H, E52L and E52Y;
(c) a substitution at position 76 selected from K76R, K76A, K76E, K76G, K76V, K76F, K76M, K76W, K76P, K76Q, K76I, K76S, K76T, K76N, K76D, K76H, K76L and K76Y;
(d) a substitution at position 78 selected from F78G, F78A, F78K, F78V, F78E, F78M, F78W, F78P, F78Q, F78I, F78S, F78T, F78N, F78D, F78R, F78H, F78L and F78Y;
(e) a substitution at position 82 selected from P82Y, P82E, P82G, P82V, P82F, P82M, P82W, P82K, P82Q, P82I, P82S, P82T, P82N, P82D, P82R, P82H, P82L and P82A;
(f) a substitution at position 28 selected from I28P, I28A, I28E, I28G, 128V, I28F, I28M, I28W, 128Q, I28K, I28S, 128T, I28N, I28D, I28R, 128H, I28L and I28Y; and
(g) a substitution at position 122 selected from I122Y, I122E, I122G, I122V, I122F, I122M, I122W, I122P, I122Q, I122K, I122S, I122T, I122N, I122D, I122R, I122H, I122L and I122A.
(h) a substitution at position 13 selected from Q13P, Q13A, Q13E, Q13G, Q13V, Q13F, Q13M, Q13W, Q13I, Q13K, Q13S, Q13T, Q13N, Q13D, Q13R, Q13H, Q13L and Q13Y; and
(i) a substitution at position 42 selected from F42P, F42A, F42E, F42G, F42V, F42Q, F42M, F42W, F42I, F42K, F42S, F42T, F42N, F42D, F42R, F42H, F42L and F42Y;
(j) a substitution at position 84 selected from D84P, D84A, D84E, D84G, D84V, D84F, D84M, D84W, D84I, D84K, D84S, D84T, D84N, D84Q, D84R, D84H, D84L and D84Y;
(k) a substitution at position 88 selected from N88P, N88A, N88E, N88G, N88V, N88F, N88M, N88W, N88I, N88K, N88S, N88T, N88Q, N88D, N88R, N88H, N88L and N88Y;
(l) a substitution at position 91 selected from V91P, V91A, V91E, V91G, V91Q, V91F, V91M, V91W, V91I, V91K, V91S, V91T, V91N, V91D, V91R, V91H, V91L and V91Y; and
(m) a substitution at position 92 selected from 192Y, 192E, 192G, 192V, 192F, 192M, 192W, 192P, 192Q, 192K, 192S, 192T, 192N, I92D, I92R, 192H, 192L and I92A;
(n) a substitution at position 94 selected from L94P, L94A, L94E, L94G, L94V, L94F, L94M, L94W, L94I, L94K, L94S, L94T, L94N, L94D, L94R, L94H, L94Q and L94Y;
(o) a substitution at position 95 selected from E95G, E95A, E95K, E95V, E95F, E95M, E95W, E95P, E95Q, E95I, E95S, E95T, E95N, E95D, E95R, E95H, E95L and E95Y;
(p) a substitution at position 119 selected from N119P, N119A, N119E, N119G, N119V, N119F, N119M, N119W, N119I, N119K, N119S, N119T, N119Q, N119D, N119R, N119H, N119L and N119Y;
(q) a substitution at position 123 selected from T123G, T123A, T123K, T123V, T123F, T123M, T123W, T123P, T123Q, T123I, T123S, T123E, T123N, T123D, T123R, T123H, T1232L and T123Y.

In some embodiments, the IL-2 variant comprises or consists of the amino acid sequence as shown in any of SEQ ID Nos: 2-13, 79-103 and 111-114.

The polypeptide complex as disclosed herein may be IL-2 bivalent fusion proteins. In some embodiments, the polypeptide complex comprises two IL-2 variants in two chains respectively, each chain comprises from N terminal to C terminal one IL-2 variant operably linked to one dimerization domain.

The polypeptide complex as disclosed herein may be bifunctional fusion proteins. In some embodiments, the polypeptide complex further comprises one or more antigen-binding moieties. The antigen-binding moiety may be in the format of a Fab, Fab′, VHH or scFv.

In some embodiments, the polypeptide complex comprises one IL-2 variant and one antigen-binding moiety in Fab format, the polypeptide complex comprises two heavy chains and one light chain, wherein from N-terminal to C-terminal:
the first heavy chain comprises the IL-2 variant operably linked to the first dimerization domain;
the second heavy chain comprises heavy chain of the Fab operably linked to the second dimerization domain; and
the light chain comprises light chain of the Fab.

In some embodiments, the polypeptide complex comprises one IL-2 variant and two antigen-binding moieties in VHH format, the polypeptide complex comprises two chains, wherein from N-terminal to C-terminal:
the first chain comprises the IL-2 variant operably linked to the first dimerization domain; and
the second chain comprises two VHHs in tandem operably linked to the second dimerization domain.

In some embodiments, the polypeptide complex comprises one IL-2 variant and one antigen-binding moiety in VHH format, the polypeptide complex comprises two chains, wherein from N-terminal to C-terminal:
the first chain comprises the IL-2 variant operably linked to the first dimerization domain; and
the second chain comprises the VHH operably linked to the second dimerization domain.

In some embodiments, the polypeptide complex comprises two IL-2 variants and two antigen-binding moieties in Fab format, the polypeptide complex comprises two heavy chains and two light chains, wherein from N-terminal to C-terminal:
the heavy chain comprises the IL-2 variant operably linked to the first or second dimerization domain, which is operably linked to heavy chain of the Fab; and
the light chain comprises light chain of the Fab.

In some embodiments, the polypeptide complex comprises two IL-2 variants and two antigen-binding moieties in VHH or scFv format, the polypeptide complex comprises two chains, wherein from N-terminal to C-terminal:
each chain comprises the IL-2 variant operably linked to the first or second dimerization domain, which is operably linked to the VHH or scFv.

In some embodiments, the antigen-binding moiety specifically binds to an antigen selected from a Tumor associated antigen (TAA), I/O checkpoints, tumor microenvironment targets, autoimmune associated targets and inflammatory disease associated targets, including but not limited to PD-1, PD-L1, PD-L2, CTLA-4, LAG3, TIM-3, TIM4, 4-1BB, OX-40, OX-40L, GITR, A2aR, TIGIT, CD96, PVRIG, CD226, 5T4, VISTA, VSIG3, VSIG4, ICOS, CD28, CD3, CD4, CD8, CD45, CD44v6, CD27, CD47, SIRPA α, SLAMF7, CD24, Siglec10, Siglec15, Siglec8, VSIR, VSIG4, PSGL-1, C5AR1, BTN1A1, BTN3A1, CD70, RANKL, CSFIR, CSF2RB, TNFRSF1/1a/1b, BDCA2, BTLA, C5aR, NKG2A, NKG2D, NKp30, NKp46, CD16a, CD56, CD166, FCGR3, CD2, Neurophilin-1, CCR8, CCR2, CCR4, CCR5, CCR6, CCR7, CCR8, GCGR, CXCR2, CXCR4, CXCR5, CALCRL, ETAR, GLP1R, CX3CR1, GPR1, GPR17, GPR20, GPR30, GPR34, GPR-65, GPCR78, GPRC5D, GPR84, LGR4, LGR5, VEGF, VEGFR, HER2, HER3, Trop2, pCAD, ER α, EGFR, de2-7 EGFR, EGFRVIII, PSMA, PSCA, PSA, TAG-72, SEZ6, SEZ6L, SEZ6L2, SEMA4D, DLL3, GD2, GPC3, KLB, KLRB1, KLRG1, GPC1, PCSK9, EpCAM, p-Cadherin, Caludin 6, Caludin 18.2, FGFR2b, FGFR3, FGFR4, MUC1, MUC13, MUC16, MUC17, MUCL3, FolRa, TfR, TF, TFR, TFPI, c-Met, NY-ESO-1, GUCY2C, LIV-1, Integrin α v β 6, Integrin α10β1, Intergrin a3, Integrin α5β4, Integrin αvβ3, Integrin αvβ8, ROR1, ROR2, PRLR, PTK7, B7-H3, Nectin-4, NetG1, Ax1, CD147, LRRC15, Napi2b, STEAP1, LY6G6D, LYPD1, MACRO, MerTK, MICA, MICB, MSLN, Mkars, G12D, CDH3, CDH6, CDH17, APLA2, CAIX, CD46, CD47, CLDN6, EphA3, Fucosyl-GM1, ITGA3, Kallikrein, MISRII, Podocalyxin, RON, ROBO1, PAUF, PLA2, Podocalyxin, PRLR, PTK7, TM4SF1, TMEFF2, TREAKR, TREM-1, TREM-2, uPARAP, TYRP1, KAAG1, RU2AS, CD146, CD63, Endoglin, Globo H, IGF-1R, TEM1, TEM8, TAXIBP3, ADAM-9, ENPP3, EphA2, EphA3, FcRH5, NaPi3b, TWEAK, DLK1, SORT1, SSTR2, STEAP1, CD25, CD39, GARP, LRRC33, LAIRA, LAMP3, LAP, LEPR, LILRB1, LILRB2, LILRB4, RAGE, FGL1, TPBG, PDGFRB, TGFBR2, CEACAMI, CEACAM5, CEACAM6, Carcinoembryonic antigen (CEA), ICAM1, A33, CAMPATH-1 (CDw52), Carboanhydrase IX (MN/CA IX), CD248, PDPN, ITGB1, ITGAV, CD20, CD19, CD21, CD22, CLL, BCMA, DCLK1, DDR1, DLK1, DPEP3, DKK1, CD5, CD13, CD30, CD33, CD34, CD36, CD37, CD38, CD43, CD52, CD55, CD94, CD99, CD7, CD71, CD73, CD74, CD79a, CD79b, CD229, CD132, CD133, G250, CSFIR (CD115), HLA-DR, HLA-G, HTRA1, TRA-1-60, IGFR, IL-2 receptor, MCSP (Melanoma-associated cell surface chondroitin sulphate proteoglycane), ART1, ASGR1, B7H3, B7-H4, B7H6, CD124, c-Kit (CD117), CD7, Clex12A, Clever-1, IL-13RA2, IL-11RA, IL-31RA, IL-4RA, IFNAR, ActRIIb, IL-7R, SLAMF7, Fms-like tyrosine kinase 3 (FLT-3, CD135), GFRA1, BTLA, GloboH, CSF2RB, chondroitin sulfate proteoglycan 4 (CSPG4, melanoma-associated chondroitin sulfate proteoglycan), ITGA4, Clec5a, Clec7a, Clec9a, Clec12a, CLEC14, CD205, CD206, CD200R1, CD228, CD229, CD40, CD40L, FcRn, TLR8, TLR9, TNFR2, LTBR, CD44, CD93, PDGF, PDGFR-alpha (CD140a), PDGFR-beta (CD140b), CD146, CD147, CRTH2, TNF-α, TGF-β, ILIRAcP, TSLP, DR5, ST2, fibroblast activating protein (FAP), CDCP1, Derlin1, Tenascin, frizzled 1-10, the vascular antigens VEGFR2 (KDR/FLK1), VEGFR3 (FLT4, CD309), Endoglin, Tie2.

In some embodiments, the antigen-binding moiety is a PD-1 binding Fab or PD-1 binding VHH. The polypeptide complexes thus formed may also be designated as PD-1/IL-2 fusion proteins.

In some embodiments, the first dimerization domain is one chain of an immunoglobulin Fc region, and the second dimerization domain is the other chain of the immunoglobulin Fc region, optionally, the Fc region further comprises a partial or whole hinge region.

In some embodiments, the Fc region is an IgG4, IgG1, IgG2 or IgG3 Fc region, and optionally comprises one or more substitutions compared to wild type human Fc to promote heterodimerization or homodimerization, to extend half-life or to remove N-glycosylation.

In some embodiments, the Fc region is selected from:
(a) a human IgG1 Fc region, optionally engineered to comprise one or more of the following: L234A/L235A mutations, M252Y/S254T/T256E mutations, G236R/L328R mutations and a “knob into hole” structure;
(b) a human IgG4 Fc region, optionally engineered to comprise one or more of the following: S228P mutation, F234A/L235A mutations, M252Y/S254T/T256E mutations and a “knob into hole” structure.

In some embodiments, the IL-2 variant and/or the antigen-binding moiety is operably linked to the Fc region via a linker, optionally the linker is a GS linker, such as (G4S) n linker, n is an integer ≥ 0, such as 0-30, 0-20, 0-15, 0-10 and 0-5.

In some embodiments, the polypeptide complex has an improved stability compared to an otherwise identical polypeptide complex comprising wild-type IL-2 instead of the IL-2, wherein stability is one or more selected from thermostability (e.g. measured by DLS), serum stability, extended serum half-life (e.g. by pharmacokinetics analysis) and structural stability.

In some embodiments, the polypeptide complex comprises an amino acid sequence of any of SEQ ID NOs: 32, 29, 30 and 31.

In some embodiments, the polypeptide complex comprises:
a first heavy chain comprising the amino acid sequence of any of SEQ ID NOs: 52-56; a second heavy chain comprising the amino acid sequence of SEQ ID NO: 57; and a light chain comprising the amino acid sequence of SEQ ID NO: 58.

In some embodiments, the polypeptide complex comprises:
a first chain comprising the amino acid sequence of any of SEQ ID NOs: 59-74, 76-78 and 104-110; and a second chain comprising the amino acid sequence of SEQ ID NO: 75.

In some embodiments, the polypeptide complex comprises:
a first heavy chain comprising the amino acid sequence of any of SEQ ID Nos: 17-28;
a second heavy chain comprising the amino acid sequence of SEQ ID NO: 33; and
a light chain comprising the amino acid sequence of SEQ ID NO: 34.

In some embodiments where the polypeptide complex is a homodimer comprising two chains, the composition comprises a nucleic acid molecule encoding one chain of the polypeptide complex as active ingredient. Specifically, the nucleic acid molecule may encode the amino acid sequence of any of SEQ ID NOs: 32, 29, 30 and 31.

In some embodiments where the polypeptide complex is a heterodimer comprising two heavy chains and one light chain, the composition comprises three nucleic acid molecules encoding each chain of the polypeptide complex respectively as active ingredient. Specifically, the nucleic acid molecules may encode: the amino acid sequence of any of SEQ ID NOs: 19-28, the amino acid sequence of SEQ ID NO: 33, and the amino acid sequence of SEQ ID NO: 34, respectively.

In some embodiments where the polypeptide complex is a heterodimer comprising two heavy chains and one light chain, the composition comprises three nucleic acid molecules encoding each chain of the polypeptide complex respectively as active ingredient. Specifically, the nucleic acid molecules may encode: the amino acid sequence of any of SEQ ID NOs: 52-56, the amino acid sequence of SEQ ID NO: 57, and the amino acid sequence of SEQ ID NO: 58, respectively.

In some embodiments where the polypeptide complex is a heterodimer comprising two heavy chains and one light chain, the composition comprises three nucleic acid molecules encoding each chain of the polypeptide complex respectively as active ingredient. Specifically, the nucleic acid molecules may encode: the amino acid sequence of any of SEQ ID NOs: 59-74, 76-78, 104-110, the amino acid sequence of SEQ ID NO: 75, respectively.

In some aspects, the present disclosure provides a polypeptide complex comprising an interleukin-2 (IL-2) variant domain, a first dimerization domain and a second dimerization domain, wherein the IL-2 variant domain comprises any of the amino acid sequences as set forth in SEQ ID Nos: 81-103 and 112-114.

In some embodiments, the polypeptide complex comprises one IL-2 variant and two antigen-binding moieties in VHH format, wherein the polypeptide complex comprises two chains, from N-terminal to C-terminal:
the first chain comprises the IL-2 variant operably linked to the first dimerization domain; and the second chain comprises two VHHs in tandem operably linked to the second dimerization domain.

In some embodiments, the polypeptide complex comprises: a first chain comprising the amino acid sequence of any of SEQ ID Nos: 59-74, 76-78 and 104-110; and a second chain comprising the amino acid sequence of SEQ ID NO: 75.

In some aspects, the present disclosure provides an isolated nucleic acid molecule, comprising a nucleic acid sequence encoding the polypeptide complex disclosed herein.

In some aspects, the present disclosure provides a vector or host cell comprising the nucleic acid molecule disclosed herein.

In some aspects, the present disclosure provides a method of improving the stability and/or pharmacokinetics properties (preferably also attenuating the binding of the IL-2 polypeptide to IL-2Rβ/γc, to IL-2Rα, or to both and the combined IL-2Rα/β/γc complex) of IL-2 polypeptide compared to wild-type IL-2, comprising:
(a) introducing a substitution at one or more positions selected from positions 32, 129, 13, 18, 19, 20, 22, 28, 38, 42, 52, 71, 76, 78, 82, 84, 87, 88, 89, 91, 92, 94, 95, 110, 119, 122, 123, 125, and 126 of the amino acid sequence of SEQ ID NO: 1 and truncating 1-20 amino acids from the C terminal;
(b) optionally, fusing the IL-2 polypeptide to a non-IL-2 moiety that extends its half-life.

In some embodiments, the substitution at position 32 is selected from K32D, K32A, K32E, K32G, K32V, K32F, K32M, K32W, K32P, K32Q, K32I, K32S, K32T, K32N, K32R, K32H, K32L and K32Y, preferably the K residue is substituted by an amino acid residue with opposite charge.

In some embodiments, the method comprises truncating 20, 15, 10, 9, 8, 7, 6, 5, 4, 3, 2 or 1 amino acid(s) from the C terminal in relation to SEQ ID NO: 1.

In some embodiments, the method further comprises introducing a substitution at position 129 selected from I129L, I129A, I129E, I129G, I129V, I129F, I129M, I129W, I129P, I129Q, I129K, I129S, I129T, I129N, I129D, I129R, I129H and I129Y.

In some embodiments, the non-IL-2 moiety is selected from PEG, lipids, an immunoglobulin Fc region, human serum albumin (HSA) and anti-HSA moiety.

In some embodiments, the stability is one or more selected from thermostability, serum stability, extended serum half-life and structural stability, and the pharmacokinetics properties includes serum half-life in vivo and clearance rate.

In some embodiments, the non-IL-2 moiety further comprises an antigen-binding moiety.

In some embodiments, the method as disclosed herein obtains an IL-2 comprising polypeptide complex that comprises an amino acid sequence of any of SEQ ID NOs: 32, 29, 30 and 31.

In some embodiments, the method as disclosed herein obtains an IL-2 comprising polypeptide complex that comprises two heavy chains and one light chain, wherein the first heavy chain comprises an amino acid sequence of any of SEQ ID NOs: 19-28; the second heavy chain comprises an amino acid sequence of SEQ ID NO: 33; and the light chain comprises an amino acid sequence of SEQ ID NO: 34.

In some embodiments, the method as disclosed herein obtains an IL-2 comprising polypeptide complex that comprises two heavy chains and one light chain, wherein the first heavy chain comprises an amino acid sequence of any of SEQ ID NOs: 52-56; the second heavy chain comprises an amino acid sequence of SEQ ID NO: 57; and the light chain comprises an amino acid sequence of SEQ ID NO: 58.

In some embodiments, the method as disclosed herein obtains an IL-2 comprising polypeptide complex that comprises two chains, wherein the first chain comprises an amino acid sequence of any of SEQ ID NOs: 59-74, 76-78, 104-110; the second chain comprises an amino acid sequence of SEQ ID NO: 75.

In some embodiments, the method as disclosed herein obtains an IL-2 comprising polypeptide complex that comprises two chains, wherein the first chain comprises an amino acid sequence of any of SEQ ID NOs: 59-74, 76-78, 104-110; the second chain comprises a heavy chain of an anti-PD-1 antibody. In some embodiments, the IL-2 is an IL-2 variant as disclosed herein.

Preferably, the polypeptide complexes as disclosed herein comprise one or more following properties:
(a) an improved stability compared to an otherwise identical polypeptide complex comprising wild-type IL-2 instead of the IL-2, wherein stability is one or more selected from thermostability (e.g. measured by DLS), serum stability, extended serum half-life (e.g. by pharmacokinetics analysis) and structural stability;
(b) a reduced binding affinity to at least one of IL-2Rα, IL-2Rβ/γc and IL-2Rα/β/γc complex compared to an otherwise identical polypeptide complex comprising wild type IL-2 instead of the IL-2 variant; and
(c) an attenuated activity compared to an otherwise identical polypeptide complex comprising wild-type IL-2 instead of the IL-2.

In some aspects, the present disclosure provides a method of modulating an immune response in a subject, comprising administering the composition as disclosed herein to the subject, optionally the immune response is NK cell, CD8+ cell, or CD4+ T cell (especially Treg) related.

In some aspects, the present disclosure provides a method for treating or preventing cancer in a subject, comprising administering an effective amount of the composition as disclosed herein to the subject.

In some embodiments, the method further comprises administering an additional anti-tumor therapy, such as cell immunotherapy including tumor-infiltrating lymphocyte (TIL) therapy, T cell receptor (TCR) therapy, chimeric antigen receptor (CAR) T cell therapy, macrophage cell therapy, and NK cell therapy, targeted therapy, chemotherapy and gene therapy (e.g. a gene therapy that uses lentivirus, AAV, poxvirus, herpes zoster virus, oncolytic virus, or other RNA/DNA vectors).

In some embodiments, the cancer is selected from colon cancer, breast cancer, lung cancer (such as NSCLC), ovarian cancer, melanoma, bladder cancer, renal cell carcinoma, liver cancer, prostate cancer, stomach cancer, pancreatic cancer, lymphoma (such as non-Hodgkin's lymphoma and diffuse large B-cell lymphoma), leukemia (such as chronic lymphocytic leukemia) and multiple myeloma, optionally the cancer is colon cancer. In some embodiments, the cancer is a PD-1 related cancer.

In some aspects, the present disclosure provides a method for treating or preventing an autoimmune disease or an inflammatory disease in a subject, comprising administering an effective amount of the composition as disclosed herein to the subject.

In some embodiments, the autoimmune or inflammatory disease is selected from inflammatory bowel disease, multiple sclerosis, rheumatoid arthritis, systemic lupus erythematosus, aplastic anemia, coeliac disease, type1 diabetes, graves' disease, psoriasis, scleroderma.

In some aspects, the present disclosure provides use of the composition as disclosed herein in the manufacture of a medicament for treating or preventing cancer, autoimmune diseases, or inflammatory diseases.

In some aspects, the present disclosure provides the composition as disclosed herein for use in treating or preventing cancer, autoimmune diseases, or inflammatory diseases.

In some aspects, the present disclosure provides a kit, comprising a container comprising the composition as disclosed herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates a schematic description of various fusion protein and polypeptide complex formats comprising wild type IL-2 or an IL-2 variant, according to some embodiments of the disclosure. The conic shape indicates the IL-2 moiety, the ladder shape indicates the hinge region, and the oval shape indicates the antigen-binding moiety and Fc region. (a, b) one IL-2 moiety+one antigen moiety (a, Fab; b, VHH) both at N-terminal; (c) two IL-2 moieties at the N-terminal; (d, e, f) two IL-2 moieties at N-terminal+two antigen moieties (d, Fab; e, VHH; f, scFv) at C-terminal; (g) one IL-2 moiety+two VHHs both at N-terminal. The F114 format comprises two Fabs at the N terminal and one IL-2 at the C terminal. These formats can accommodate different antigen-binding moieties such as Fabs, VHHs and scFvs comprising variable regions targeted to different epitopes/antigens.

FIG. 2 illustrates IL-2 variants showed different potency in primary human CD8+ T cells, determined by STAT5 phosphorylation assay.

FIG. 3 illustrates IL-2 variants showed different potency in activated CD8+T (a) and primary human CD8+ T cells (b), determined by STAT5 phosphorylation assay.

FIG. 4 illustrates serum stability of bivalent IL-2 variants Z20-1 (a), BMK8 (b), Z20-4 (c) and Z20-5 (d), which showed different potency in human activated CD8+ T cells, as determined by STAT5 phosphorylation assay.

FIG. 5 illustrates the serum concentration profiles of Z20-1, Z20-5 and BMK7 in C57BL/6 mice, detected by ELISA of Fc-Fc.

FIG. 6 illustrates modeling of interaction between receptors and T2U0.E44-49 (a), and between receptors and Z20-5 (b).

FIG. 7 illustrates the change in tumor volume of MC38 colon carcinoma model after treated with IL-2 variants.

FIG. 8 illustrates the IHC of lungs from MC38 colon carcinoma model after treated with IL-2 variants, arrow noted inflammatory cells infiltration.

FIG. 9 illustrates PD-1/IL-2 fusion proteins showed similar binding affinity on CHO-PD1 engineered cell line.

FIG. 10 illustrates PD-1/IL-2 fusion proteins showed different potency in activated CD8+T (a) and primary human CD8+ T cells (b), determined by STAT5 phosphorylation assay.

FIG. 11 illustrates PD-1/IL-2 fusion proteins showed different potency in activated CD8+T (a) and primary human CD8+ T cells (b), determined by STAT5 phosphorylation assay.

FIG. 12 illustrates PD-1/IL-2 fusion proteins showed different potency in activated CD8+T determined by STAT5 phosphorylation assay.

FIG. 13 illustrates the change in tumor volume of CT-26 colon carcinoma model after treated with PD-1/IL-2 fusion proteins.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

### Definitions

In order to better understand the disclosure, the definitions and explanations of the relevant terms are provided as follows.

The term “IL-2” or “Interleukin-2”, as used herein, is intended to encompass any form of IL-2, for example, 1) native unprocessed IL-2 molecule, “full-length” IL-2 protein or naturally occurring variants of IL-2; 2) any form of IL-2 that results from processing in the cell; or 3) full length, a fragment (e.g., a truncated form) or a modified form. IL-2 is a cytokine mainly produced from activated T cells and contributes to proliferation and activation of various immune cells. Human mature IL-2 has a molecular weight of about 15 kDa (133 amino acids, as shown in SEQ ID NO: 1) and has a four-α-helix bundle structure. As used in the disclosure, “IL-2” may be either wild-type IL-2 or an IL-2 variant, and “IL-2 domain” comprises a wild-type IL-2 or an IL-2 variant peptide.

The term “variant”, with regard to polypeptide or protein, means a biologically active polypeptide which includes one or more amino acid mutations in the native protein sequence. Optionally, the one or more amino acid mutations include amino acid substitution, deletion, and/or insertion at certain positions in the amino acid sequence. A variant has at least about 80%, and preferably at least about 85%, more preferably at least about 90%, and even more preferably at least about 95% (e.g. at least 96%, 97%, 98%, or 99% or higher) amino acid sequence identity with the corresponding native sequence polypeptide. Such variants include, for instance, polypeptides wherein one or more amino acid (naturally occurring amino acid and/or a non-naturally occurring amino acid) residues are added, or deleted, at the N- and/or C-terminus of the polypeptide. Variants also include polypeptide fragments (e.g., subsequences, truncations, etc.), typically biologically active, of the native sequence.

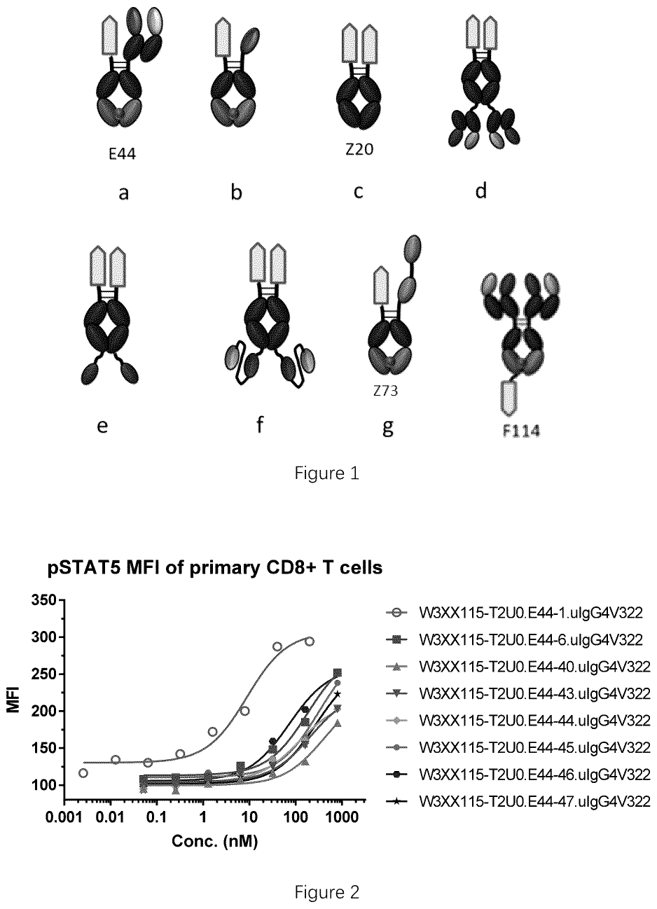

The term “IL-2 variant”, as used herein, includes all proteins which are produced by adding any modifications to wild-type IL-2, and has a function similar to wild-type IL-2, such as a specific binding to IL-R2a, IL-2Rβ/γc, and/or the combined IL-R2α/β/γc complex (although preferably the binding affinity is attenuated compared to the wild-type IL-2), activation of immune cells such as T cells (e.g. CD8+ T cells, NK cells, and Treg cells), phosphorylation of STAT5, among others. Examples of the variants include an IL-2 variant in which the wt IL-2 is modified by an amino acid modification (for example, deletion, substitution or addition), an IL-2 variant in which the wt IL-2 is modified by saccharide modification, and IL-2 variant in which the wt IL-2 is modified by chemical modification. Depending on the context, the term “IL-2 variant” may also refer to a polypeptide complex comprising the IL-2 variant, e.g. in E44, Z20 or Z73 format as shown in FIG. 1.

The term “antibody” or “Ab” herein is used in the broadest sense, which encompasses various antibody structures, including polyclonal antibodies, monospecific and multispecific antibodies (e.g. bispecific antibodies) and the polypeptide complexes as disclosed herein. A native intact antibody generally is a Y-shaped tetrameric protein comprising two heavy (H) and two light (L) polypeptide chains held together by covalent disulfide bonds and non-covalent interactions. Light chains of an antibody may be classified into k and A light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), Chothia & Lesk (1987) J. Mol. Biol. 196:901-917; Chothia et al., (1989) Nature 342:878-883, Contact numbering, AbM numbering or IMGT numbering. Antibodies may be of different antibody isotypes, for example, IgG (e.g., IgG1, IgG2, IgG3 or IgG4 subtype), IgA1, IgA2, IgD, IgE or IgM antibody. In a broad sense, the polypeptide complexes as disclosed herein comprising an antigen-binding moiety also belongs to an antibody.

The term “antigen-binding moiety” as used herein refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)2, an Fv fragment, a disulphide stabilized Fv fragment (dsFv), a (dsFv)2, a bispecific dsFv (dsFv-dsFv′), a disulphide stabilized diabody (ds diabody), a multispecific antibody, a camelized single domain antibody, a nanobody, a domain antibody, a VHH, a bivalent domain antibody and a TCR. The term “Fab”, as used herein, is meant the polypeptide that comprises the VH, CH1, VL and CL immunoglobulin domains. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in (Spiess et al., Molecular Immunology, 67 (2), pp. 95-106 (2015), and Brinkman et al., mAbs, 9 (2), pp. 182-212 (2017), which are incorporated herein by their entirety.

The term “hinge region”, as used herein, has a same meaning as used with regard to an antibody, which refers to a short sequence of the heavy chains (H) of immunoglobulins linking the Fab (Fragment antigen binding) region to the Fc (Fragment crystallizable) region. The hinge region may be a full or a partial hinge region. The hinge region may consist of at least 2 (e.g., 5, 10, 15, 20, 40, 60 or more) amino acids which result in a flexible or semi-flexible linkage between adjacent variable and/or constant domains in a single polypeptide molecule. In some embodiments, the hinge region comprised in the polypeptide complex as disclosed herein is a C-terminal or N-terminal truncated hinge region. It will be appreciated that the hinge region is a specific linker and may be suitably replaced by other linker sequences in constructing the fusion protein or polypeptide complex herein.

The term “Fc”, as used herein, has a same meaning as used with regard to an antibody, which refers to that portion of the antibody comprising the second (CH2) and third (CH3) constant regions of a first heavy chain bound to the second and third constant regions of a second heavy chain via disulfide bonding. The Fc region of the antibody is responsible for various effector functions such as ADCC and CDC, but generally does not function in antigen binding. In the present disclosure, the term “Fc” includes both wild-type Fc, Fc variants and grafted Fc.

The term “modification”, with respect to an amino acid residue/position as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more amino acids adjacent to said residue/position, and deletion of said residue/position. An “amino acid substitution”, or variation thereof, refers to the replacement of an existing amino acid residue in a predetermined (starting) amino acid sequence with a different amino acid residue. Generally, the modification results in alteration in at least one physicobiochemical activity of the variant polypeptide compared to a polypeptide comprising the starting (or “wild type”) amino acid sequence. For example, in an IL-2 variant, a physicobiochemical activity that is altered can be binding affinity, binding capability and/or binding effect upon a target molecule. As used herein, two or more substitutions in an amino acid sequence may be expressed with “+” or “/” between each substitution.

As used herein, the term “fusion protein” refers to a chimeric polypeptide comprising an IL-2 moiety and a non-IL-2 moiety (and optionally more portions) operably linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the non-IL-2 moiety can extend the half-life of IL-2 in vivo. In some embodiments, the non-IL-2 moiety is an immunoglobulin constant region comprising the hinge region and Fc region, thus the generated fusion protein is referred to as IL-2/Fc fusion protein. The IL-2/Fc fusion protein comprises the IL-2 moiety operably linked to the Fc region (optionally linked via a hinge region), and generally is a dimer.

The term “polypeptide complex” as used herein refers to a polypeptide complex comprising an IL-2 domain operably linked to a dimerization domain such as an immunoglobulin Fc region (i.e. IL-2/Fc polypeptide complex). Such polypeptide complexes are structurally similar to a conventional antibody, in view that the IL-2 domain replaces the Fab or VHH in a conventional antibody present on two antigen-binding arms. The term “bivalent” as used herein means there are two IL-2 domains present in the polypeptide complex or fusion protein. The term “monovalent” as used herein means there are one IL-2 domain present in the polypeptide complex or fusion protein.

The terms “operably linked” as used herein refer to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antigen-binding moiety may be operably linked to a Fc region so as to provide for a stable product with antigen-binding activity. By “operably linked to”, the antigen-binding moiety may be directly linked to the Fc region as long as the two parts can function normally, or more preferably, the antigen-binding moiety may be indirectly linked to the Fc region via a linker sequence, such as a hinge region. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “gene therapy”, as used herein, refers to a therapy that aims to treat diseases by replacing, inactivating or introducing genes into cells-either inside the body (in vivo) or outside of the body (ex vivo). In some embodiments, a DNA or RNA sequence encoding the IL-2 variant(s) as disclosed herein is administered to a subject. Gene therapy includes using lentivirus, AAV, poxvirus, herpes zoster virus, oncolytic virus and other RNA/DNA vectors to deliver the DNA or RNAs encoding the IL-2 variants.

The term “cell therapy”, as used herein, refers to a therapy that aims to treat diseases by restoring or altering certain sets of cells or by using cells to carry a therapy through the body. With cell therapy, cells are cultivated or modified outside the body before being injected into the patient. The cells may originate from the patient (autologous cells) or a donor (allogeneic cells). Current cell therapy includes using CAR-T, TCR-T, TIL, CAR-NK, CAR-γδT, CAR-macrophage and other engineered immune cells. In some embodiments herein, the treatment method further includes the cell therapy.

The term “EC₅₀,” as used herein, which is also termed as “half maximal effective concentration” refers to the concentration of a drug, antibody or toxicant which induces a response halfway between the baseline and maximum after a specified exposure time.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as 2 phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, a vector encoding the heavy chain and/or light chain of the polypeptide complex was transfected into 293F cells.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017 Nov. 9). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “antibody-dependent cell-mediated cytotoxicity” or “ADCC,” as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The term “subject” includes any human or nonhuman animal, preferably humans.

The term “cancer,” as used herein, refers to any tumor or a malignant cell growth, proliferation or metastasis-mediated, solid tumors and non-solid tumors such as leukemia, which can initiate a medical condition.

The term “autoimmune disease,” as used herein, refers to any conditions arising from an abnormal immune response to a functioning body part such as rheumatoid arthritis, systemic lupus erythematosus, inflammatory bowel disease, multiple sclerosis, which can initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of diseases or conditions such as cancers, refers to an active agent, a drug or an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

### IL-2 Variants

In some aspects, the present disclosure provides IL-2 variants which comprise one or more modification(s), e.g. insertion, substitution and/or deletion, compared to the wild-type IL-2 protein, such as human wild-type IL-2 protein. The mature form of human wild-type IL-2 protein is exemplified in SEQ ID NO: 1.

In some embodiments, the IL-2 variant has a truncation of one or more amino acids from the C terminal of the wild-type IL-2. Said truncation of one or more amino acids may be a truncation of 1, 2, 3, 4, 5, 6, 7, 8, 9, 10, 15, 20 or more amino acid(s), as long as the IL-2 variant can retain (preferably, attenuate) the binding capacity to IL-R2a, to IL-2Rβ/γc, and/or to the combined IL-2Rα/β/γc complex. The IL-2 variant may comprise an amino acid sequence that differs from SEQ ID NO: 1 only by a C-terminal truncation. In some specific embodiments, the amino acid sequence of the IL-2 variant is as set forth in SEQ ID NO: 2.

In some embodiments, the IL-2 variant has at least one amino acid substitution compared to SEQ ID NO: 1. The substitution(s) may occur at the amino acids involved in stability of IL-2. Specifically, the substitution may occur at one or more of positions 3, 13, 18, 19, 20, 22, 28, 32, 42, 52, 72, 71, 76, 78, 82, 84, 87, 88, 91, 92, 94, 110, 119, 122, 123, 125, 126 and 129 corresponding to SEQ ID NO: 1. In some specific embodiments, the amino acid sequence of the IL-2 variant is as set forth in SEQ ID NO: 3-13, 79-103 and 111-114.

In some embodiments, the IL-2 variant has at least one amino acid substitution compared to SEQ ID NO: 1. The substitution(s) may occur at the amino acids located the IL-2/Ra or IL-2Rβ/γc binding interface. Specifically, the substitution may occur at one or more of positions 110, 122 and 129 corresponding to SEQ ID NO: 1. Preferably, in relation to SEQ ID NO: 1, the IL-2 variant comprises a substitution at one or more positions selected from positions 32, 28, 52, 76, 78 and 82 which may facilitate the stabilization of the IL-2 variant, in combination with a substitution at one or more positions selected from positions 129, 110 and 122 which may attenuate the binding of IL-2 with its receptors.

In some embodiments, the IL-2 variant has at least one amino acid substitution compared to SEQ ID NO: 1. The substitution(s) may occur at the amino acids located the IL-2/Ra or IL-2Rβ/γc binding interface. Specifically, the substitution may occur at one or more of positions 110, 122 and 129 corresponding to SEQ ID NO: 1. Preferably, in relation to SEQ ID NO: 1, the IL-2 variant comprises a substitution at one or more positions selected from 3, 13, 18, 19, 20, 22, 42, 71, 84, 87, 88, 91, 92, 94, 119, 123 and 126, in combination with a substitution at one or more positions selected from positions 129, 110 and 122 which may attenuate the binding of IL-2 with its receptors.

In some embodiments, the IL-2 variant comprises one or more substitutions at positions 3, 13, 18, 19, 20, 22, 25, 28, 32, 37, 38, 41, 42, 43, 45, 52, 61, 62, 65, 68, 71, 72, 76, 78, 82, 84, 87, 88, 91, 92, 94, 95, 107, 110, 111, 119, 122, 123, 125, 126, 127, 128 and 129 of the amino acid sequences as set forth in SEQ ID NO: 1. In some embodiments, the IL-2 variant comprises a substitution at position 28 of the amino acid sequence as set forth in SEQ ID NO: 1, the original amino acid isoleucine (I) can be substituted by any amino acid other than cysteine, such as A, D, E, F, G, H, T, K, M, N, P, Q, R, S, L, V, W, Y, more specifically P.

In some embodiments, the IL-2 variant comprises a substitution at position 32 of the amino acid sequence as set forth in SEQ ID NO: 1, the original amino acid lysine (K) can be substituted by any amino acid other than cysteine, such as A, D, E, F, G, H, T, I, M, N, P, Q, R, S, L, V, W, Y, more specifically D.

In some embodiments, the IL-2 variant comprises a substitution at position 52 of the amino acid sequence as set forth in SEQ ID NO: 1, the original amino acid glutamic acid (E) can be substituted by any amino acid other than cysteine, such as A, D, F, G, H, T, K, M, N, P, Q, R, S, L, V, W, Y, more specifically G.

In some embodiments, the IL-2 variant comprises a substitution at position 76 of the amino acid sequence as set forth in SEQ ID NO: 1, the original amino acid lysine (K) can be substituted by any amino acid other than cysteine, such as A, D, E, F, G, H, T, I, M, N, P, Q, R, S, L, V, W, Y, more specifically R.

In some embodiments, the IL-2 variant comprises a substitution at position 78 of the amino acid sequence as set forth in SEQ ID NO: 1, the original amino acid phenylalanine (F) can be substituted by any amino acid other than cysteine, such as A, D, E, K, G, H, T, I, M, N, P, Q, R, S, L, V, W, Y, more specifically G.

In some embodiments, the IL-2 variant comprises a substitution at position 82 of the amino acid sequence as set forth in SEQ ID NO: 1, the original amino acid proline (P) can be substituted by any amino acid other than cysteine, such as A, D, E, K, G, H, T, I, M, N, F, Q, R, S, L, V, W, Y, more specifically Y.

In some embodiments, the IL-2 variant comprises a substitution at position 110 of the amino acid sequence as set forth in SEQ ID NO: 1, the original amino acid glutamic acid (E) can be substituted by any amino acid other than cysteine, such as A, D, E, F, G, H, T, K, M, N, P, Q, R, S, L, V, W, Y, more specifically R, I or T.

In some embodiments, the IL-2 variant comprises a substitution at position 122 of the amino acid sequence as set forth in SEQ ID NO: 1, the original amino acid isoleucine (I) can be substituted by any amino acid other than cysteine, such as A, D, E, F, G, H, K, M, N, P, Q, R, S, T, L, V, W, Y, more specifically Y, T or V.

In some embodiments, the IL-2 variant comprises a substitution at position 129 of the amino acid sequence as set forth in SEQ ID NO: 1, the original amino acid isoleucine (I) can be substituted by any amino acid other than cysteine and isoleucine, such as A, D, E, F, G, H, T, K, M, N, P, Q, R, S, L, V, W, Y, more specifically L, V, A, S or T.

In some embodiments, the IL-2 variant comprises a substitution at position 13 of the amino acid sequence as set forth in SEQ ID NO: 1, the original amino acid glutamine (Q) can be substituted by any amino acid other than cysteine, such as A, D, E, F, G, H, T, K, M, N, P, R, S, L, V, W, Y, I, more specifically D or R.

In some embodiments, the IL-2 variant comprises a substitution at position 84 of the amino acid sequence as set forth in SEQ ID NO: 1, the original amino acid aspartic acid (D) can be substituted by any amino acid other than cysteine, such as A, E, F, G, H, T, K, M, N, P, Q, R, S, L, V, W, Y, I, more specifically K or T.

In some embodiments, the IL-2 variant comprises a substitution at position 87 of the amino acid sequence as set forth in SEQ ID NO: 1, the original amino acid serine(S) can be substituted by any amino acid other than cysteine, such as A, E, F, G, H, T, K, M, N, P, Q, R, L, V, W, Y, I, more specifically K or I.

In some embodiments, the IL-2 variant comprises a substitution at position 88 of the amino acid sequence as set forth in SEQ ID NO: 1, the original amino acid asparagine (N) can be substituted by any amino acid other than cysteine, such as A, D, E, F, G, H, T, K, M, P, Q, R, S, L, V, W, Y, I, more specifically K.

In some embodiments, the IL-2 variant comprises a substitution at position 91 of the amino acid sequence as set forth in SEQ ID NO: 1, the original amino acid valine (V) can be substituted by any amino acid other than cysteine, such as A, D, E, F, G, H, T, K, M, N, P, Q, R, S, L, W, Y, I, more specifically E or S.

In some embodiments, the IL-2 variant comprises a substitution at position 92 of the amino acid sequence as set forth in SEQ ID NO: 1, the original amino acid isoleucine (I) can be substituted by any amino acid other than cysteine, such as A, D, E, F, G, H, T, K, M, N, P, Q, R, S, L, W, Y, more specifically D and R.

In some embodiments, the IL-2 variant comprises a substitution at position 94 of the amino acid sequence as set forth in SEQ ID NO: 1, the original amino acid leucine (L) can be substituted by any amino acid other than cysteine, such as A, D, E, F, G, H, T, K, M, N, P, Q, R, S, V, W, Y, I, more specifically D.

In some embodiments, the IL-2 variant comprises a substitution at position 95 of the amino acid sequence as set forth in SEQ ID NO: 1, the original amino acid glutamic acid (E) can be substituted by any amino acid other than cysteine, such as A, D, F, G, H, T, K, M, N, P, Q, R, S, L, V, W, Y, I, more specifically R or Y.

In some embodiments, the IL-2 variant comprises a substitution at position 119 of the amino acid sequence as set forth in SEQ ID NO: 1, the original amino acid asparagine (N) can be substituted by any amino acid other than cysteine, such as A, D, E, F, G, H, T, K, M, P, Q, R, S, L, V, W, Y, I, more specifically R or Q.

In some embodiments, the IL-2 variant comprises a substitution at position 123 of the amino acid sequence as set forth in SEQ ID NO: 1, the original amino acid threonine (T) can be substituted by any amino acid other than cysteine, such as A, D, E, F, G, H, K, M, N, P, Q, R, S, L, V, W, Y, I, more specifically K or V.

In some embodiments, the IL-2 variant comprises both a truncation at the C-terminal and one or more substitution(s) selected from positions 3, 13, 18, 19, 20, 22, 28, 32, 42, 52, 72, 76, 78, 82, 84, 87, 88, 91, 92, 94, 110, 119, 122, 125, 126 and 129.

In some embodiments, the IL-2 variant comprises a C-terminal truncation of 1, 2, 3, 4, 5, 6, 7, 8, 9, or 10 amino acid(s) and a substitution at position 28, such as I28P. Specifically, the IL-2 variant may comprise a C-terminal truncation of 4 or 5 amino acid(s) and a substitution of I28P.

In some embodiments, the IL-2 variant comprises a C-terminal truncation of 1, 2, 3, 4, 5, 6, 7, 8, 9, or 10 amino acid(s) and a substitution at position 32, such as K32D. Specifically, the IL-2 variant may comprise a C-terminal truncation of 4 or 5 amino acid(s) and a substitution of K32D.

In some embodiments, the IL-2 variant comprises a C-terminal truncation of 1, 2, 3, 4, 5, 6, 7, 8, 9, or 10 amino acid(s) and a substitution at position 52, such as E52G. Specifically, the IL-2 variant may comprise a C-terminal truncation of 4 or 5 amino acid(s) and a substitution of E52G.

In some embodiments, the IL-2 variant comprises a C-terminal truncation of 1, 2, 3, 4, 5, 6, 7, 8, 9, or 10 amino acid(s) and a substitution at position 76, such as K76R. Specifically, the IL-2 variant may comprise a C-terminal truncation of 4 or 5 amino acid(s) and a substitution of K76R.

In some embodiments, the IL-2 variant comprises a C-terminal truncation of 1, 2, 3, 4, 5, 6, 7, 8, 9, or 10 amino acid(s) and a substitution at position 78, such as F78G. Specifically, the IL-2 variant may comprise a C-terminal truncation of 4 or 5 amino acid(s) and a substitution of F78G.

In some embodiments, the IL-2 variant comprises a C-terminal truncation of 1, 2, 3, 4, 5, 6, 7, 8, 9, or 10 amino acid(s) and a substitution at position 82, such as P82Y. Specifically, the IL-2 variant may comprise a C-terminal truncation of 4 or 5 amino acid(s) and a substitution of P82Y.

In some embodiments, the IL-2 variant comprises a C-terminal truncation of 1, 2, 3, 4, 5, 6, 7, 8, 9, or 10 amino acid(s) and a substitution at position 110, such as E110R, E110I, or E110T. Specifically, the IL-2 variant may comprise a C-terminal truncation of 4 or 5 amino acid(s) and a substitution of E110R, E110I, or E110T.

In some embodiments, the IL-2 variant comprises a C-terminal truncation of 1, 2, 3, 4, 5, 6, 7, 8, 9, or 10 amino acid(s) and a substitution at position 122, such as I122Y, 1122T, or I122V.

Specifically, the IL-2 variant may comprise a C-terminal truncation of 4 or 5 amino acid(s) and a substitution of I122Y, I122T, or I122V.

In some embodiments, the IL-2 variant comprises a C-terminal truncation of 1, 2, 3, 4, 5, 6, 7, 8, 9, or 10 amino acid(s) and a substitution at position 129, such as I129L, I129V, I129A, I129S or I129T. Specifically, the IL-2 variant may comprise a C-terminal truncation of 4 or 5 amino acid(s) and a substitution of I129L, I129V, I129A, I129S or I129T.

In some embodiments, the IL-2 variant comprises a C-terminal truncation of 1, 2, 3, 4, 5, 6, 7, 8, 9, or 10 amino acid(s) and a substitution at position 42, such as F42A, F42I. Specifically, the IL-2 variant may comprise a C-terminal truncation of 4 or 5 amino acid(s) and a substitution of F42A or F42I.

In some embodiments, the IL-2 variant comprises a C-terminal truncation of 1, 2, 3, 4, 5, 6, 7, 8, 9, or 10 amino acid(s) and a substitution at position 38, such as R38W. Specifically, the IL-2 variant may comprise a C-terminal truncation of 4 or 5 amino acid(s) and a substitution of R38W.

In some embodiments, the IL-2 variant comprises a C-terminal truncation of 1, 2, 3, 4, 5, 6, 7, 8, 9, or 10 amino acid(s) and substitutions at position 42, 110 and 129, such as F42I, E110R and I129L. Specifically, the IL-2 variant may comprise a C-terminal truncation of 4 or 5 amino acid(s) and a substitution of F42I, E110R and I129L.

In some embodiments, the IL-2 variant comprises a C-terminal truncation of 1, 2, 3, 4, 5, 6, 7, 8, 9, or 10 amino acid(s) and substitutions at position 32, 42 and 129, such as K32D, F42I and I129L. Specifically, the IL-2 variant may comprise a C-terminal truncation of 4 or 5 amino acid(s) and a substitution of K32D, F42I and I129L.

In some embodiments, the IL-2 variant comprises a C-terminal truncation of 1, 2, 3, 4, 5, 6, 7, 8, 9, or 10 amino acid(s) and substitutions at position 13, 32, 42 and 129, such Q13D, K32D, F42I and I129L. Specifically, the IL-2 variant may comprise a C-terminal truncation of 4 or 5 amino acid(s) and a substitution of Q13D, K32D, F42I and I129L.

In some embodiments, the IL-2 variant comprises a C-terminal truncation of 1, 2, 3, 4, 5, 6, 7, 8, 9, or 10 amino acid(s) and substitutions at position 13, 32, 42 and 129, such Q13R, K32D, F42I and I129L. Specifically, the IL-2 variant may comprise a C-terminal truncation of 4 or 5 amino acid(s) and a substitution of Q13R, K32D, F421 and I129L.

In some embodiments, the IL-2 variant comprises a C-terminal truncation of 1, 2, 3, 4, 5, 6, 7, 8, 9, or 10 amino acid(s) and substitutions at position 84, 32, 42 and 129, such D84K, K32D, F42I and I129L. Specifically, the IL-2 variant may comprise a C-terminal truncation of 4 or 5 amino acid(s) and a substitution of D84K, K32D, F42I and I129L.

In some embodiments, the IL-2 variant comprises a C-terminal truncation of 1, 2, 3, 4, 5, 6, 7, 8, 9, or 10 amino acid(s) and substitutions at position 84, 32, 42 and 129, such D84T, K32D, F42I and I129L. Specifically, the IL-2 variant may comprise a C-terminal truncation of 4 or 5 amino acid(s) and a substitution of D84T, K32D, F42I and I129L.

In some embodiments, the IL-2 variant comprises a C-terminal truncation of 1, 2, 3, 4, 5, 6, 7, 8, 9, or 10 amino acid(s) and substitutions at position 87, 32, 42 and 129, such S87R, K32D, F42I and I129L. Specifically, the IL-2 variant may comprise a C-terminal truncation of 4 or 5 amino acid(s) and a substitution of S87R, K32D, F42I and I129L.

In some embodiments, the IL-2 variant comprises a C-terminal truncation of 1, 2, 3, 4, 5, 6, 7, 8, 9, or 10 amino acid(s) and substitutions at position 88, 32, 42 and 129, such N88K, K32D, F42I and I129L. Specifically, the IL-2 variant may comprise a C-terminal truncation of 4 or 5 amino acid(s) and a substitution of N88K, K32D, F42I and I129L.

In some embodiments, the IL-2 variant comprises a C-terminal truncation of 1, 2, 3, 4, 5, 6, 7, 8, 9, or 10 amino acid(s) and substitutions at position 91, 32, 42 and 129, such V91E, K32D, F42I and I129L. Specifically, the IL-2 variant may comprise a C-terminal truncation of 4 or 5 amino acid(s) and a substitution of V91E, K32D, F42I and I129L.

In some embodiments, the IL-2 variant comprises a C-terminal truncation of 1, 2, 3, 4, 5, 6, 7, 8, 9, or 10 amino acid(s) and substitutions at position 91, 32, 42 and 129, such V91S, K32D, F42I and I129L. Specifically, the IL-2 variant may comprise a C-terminal truncation of 4 or 5 amino acid(s) and a substitution of V91S, K32D, F421 and I129L.

In some embodiments, the IL-2 variant comprises a C-terminal truncation of 1, 2, 3, 4, 5, 6, 7, 8, 9, or 10 amino acid(s) and substitutions at position 92, 32, 42 and 129, such I92D, K32D, F42I and I129L. Specifically, the IL-2 variant may comprise a C-terminal truncation of 4 or 5 amino acid(s) and a substitution of I92D, K32D, F421 and I129L.

In some embodiments, the IL-2 variant comprises a C-terminal truncation of 1, 2, 3, 4, 5, 6, 7, 8, 9, or 10 amino acid(s) and substitutions at position 92, 32, 42 and 129, such I92R, K32D, F42I and I129L. Specifically, the IL-2 variant may comprise a C-terminal truncation of 4 or 5 amino acid(s) and a substitution of I92R, K32D, F42I and I129L.

In some embodiments, the IL-2 variant comprises a C-terminal truncation of 1, 2, 3, 4, 5, 6, 7, 8, 9, or 10 amino acid(s) and substitutions at position 94, 32, 42 and 129, such L94D, K32D, F42I and I129L. Specifically, the IL-2 variant may comprise a C-terminal truncation of 4 or 5 amino acid(s) and a substitution of L94D K32D, F421 and I129L.

In some embodiments, the IL-2 variant comprises a C-terminal truncation of 1, 2, 3, 4, 5, 6, 7, 8, 9, or 10 amino acid(s) and substitutions at position 95, 32, 42 and 129, such E95R, K32D, F42I and I129L. Specifically, the IL-2 variant may comprise a C-terminal truncation of 4 or 5 amino acid(s) and a substitution of E95R, K32D, F42I and I129L.

In some embodiments, the IL-2 variant comprises a C-terminal truncation of 1, 2, 3, 4, 5, 6, 7, 8, 9, or 10 amino acid(s) and substitutions at position 95, 32, 42 and 129, such E95S, K32D, F42I and I129L. Specifically, the IL-2 variant may comprise a C-terminal truncation of 4 or 5 amino acid(s) and a substitution of E95S, K32D, F42I and I129L.

In some embodiments, the IL-2 variant comprises a C-terminal truncation of 1, 2, 3, 4, 5, 6, 7, 8, 9, or 10 amino acid(s) and substitutions at position 119, 32, 42 and 129, such N119R, K32D, F42I and I129L. Specifically, the IL-2 variant may comprise a C-terminal truncation of 4 or 5 amino acid(s) and a substitution of N119R, K32D, F42I and I129L.

In some embodiments, the IL-2 variant comprises a C-terminal truncation of 1, 2, 3, 4, 5, 6, 7, 8, 9, or 10 amino acid(s) and substitutions at position 119, 32, 42 and 129, such N119Q, K32D, F42I and I129L. Specifically, the IL-2 variant may comprise a C-terminal truncation of 4 or 5 amino acid(s) and a substitution of N119Q, K32D, F42I and I129L.

In some embodiments, the IL-2 variant comprises a C-terminal truncation of 1, 2, 3, 4, 5, 6, 7, 8, 9, or 10 amino acid(s) and substitutions at position 123, 32, 42 and 129, such T123K, K32D, F42I and I129L. Specifically, the IL-2 variant may comprise a C-terminal truncation of 4 or 5 amino acid(s) and a substitution of T123K, K32D, F42I and I129L. In some embodiments, the IL-2 variant comprises a C-terminal truncation of 1, 2, 3, 4, 5, 6, 7, 8, 9, or 10 amino acid(s) and substitutions at position 123, 32, 42 and 129, such T123V, K32D, F42I and I129L. Specifically, the IL-2 variant may comprise a C-terminal truncation of 4 or 5 amino acid(s) and a substitution of T123V, K32D, F421 and I129L.

In some specific embodiments, the amino acid sequence of the IL-2 variant is selected from those as set forth in SEQ ID NOs: 2, 17, 29 and homologous sequences thereof with at least 95% identity, for example with at least 96% identity, at least 97% identity, at least 98% identity, or at least 99% identity or higher.

In some embodiments, the IL-2 variant comprises at least two substitutions occurred at one or more of positions 3, 13, 18, 19, 22, 28, 32, 42, 52, 72, 76, 78, 82, 84, 88, 91, 94, 110, 119, 122, 125, 126 and 129 corresponding to SEQ ID NO: 1. In some specific embodiments, the amino acid sequence of the IL-2 variant is selected from SEQ ID NOs: 3-13, 18-32, 52-56, 59-74, 76-78, 79-103 and homologous sequences thereof with at least 95% identity.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. MoI. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25 (17): 3389-3402. When utilizing BLAST and Gapped BLAST programs, the default parameters of the respective programs (e.g., XBLAST and NBLAST) can be used. See www.ncbi.nlm.nih.gov.

### IL-2-Comprising Fusion Proteins and Polypeptide Complexes

In some aspects, the present disclosure provides a fusion protein comprising IL-2 fused to a non-IL-2 moiety. The incorporation of non-IL-2 moiety, such as PEGs, functional analogs of PEG, lipids and long-lived serum proteins, may serve for the purpose of half-life extension. In some embodiments, the non-IL-2 moiety is an antibody Fc region. In some other embodiments, the non-IL-2 moiety is human serum albumin (HSA). In some other embodiments, the non-IL-2 moiety is an anti-HSA moiety. The generated fusion protein may be a monomer or dimer.

As will be appreciated by those in the art, both Fc and albumin fusions achieve extended half-lives not only by increasing the size of the fusion protein, but also by taking advantage of the body's natural recycling mechanism: the neonatal Fc receptor, FcRn. The pH-dependent binding of these proteins to FcRn prevents degradation of the fusion protein in the endosome. Fusion to antibody Fc can improve the solubility and stability of the fusion protein.

A major difference between Fc and HSA/anti-HSA moiety is the dimeric nature of Fc versus the monomeric structure of has/anti-HSA moiety, leading to presentation of a fused protein as a dimer or a monomer depending on the choice of fusion partner. The dimeric nature of a Fc fusion protein can produce an avidity effect where the target receptors are spaced closely enough together or are themselves dimers.

In addition, the fusion protein as disclosed herein may further attenuate affinity to IL-2Rβ/γc, to IL-2Rα, or to both and the combined IL-2Rα/β/γc complex, through steric hindrance, hydrogen bond, salt bridge, hydrophobic effects, or other intramolecular interactions that form.

In some embodiments, the present disclosure provides a polypeptide complex comprising an IL-2 moiety, an antigen-binding moiety and a full or partial hinge region plus an Fc region. The antigen-binding moiety may be a Fab, scFv, a nanobody (VHH) or a TCR.

In some embodiments, the antigen-binding portion is Fab. The IL-2 moiety may be constructed on a different chain from the Fab. Alternatively, the IL-2 moiety may be on the same heavy chain with the VH region of the Fab. Where the IL-2 moiety is on the same heavy chain with the VH region, the IL-2 moiety can be operably linked to the hinge region and Fc region (optionally via a linker), which in turn is operably linked to the VH region, i.e. the IL-2 moiety and the Fab are separated by intervening hinge region and Fc region. In some other embodiments, the IL-2 moiety and the Fab are on the same side (usually N terminal) of the Fc region.

In some embodiments, the antigen-binding portion is VHH. The IL-2 moiety may be constructed on a different chain from the VHH. Alternatively, the IL-2 moiety may be on the same heavy chain with the VHH. Where the IL-2 moiety is on the same chain with the VHH region, the IL-2 moiety can be operably linked to the hinge region and Fc region (optionally via a linker), which in turn is operably linked to the VHH region, i.e. the IL-2 moiety and the VHH are separated by intervening hinge region and Fc region. In some other embodiments, the IL-2 moiety and the VHH are on the same side (usually N terminal) of the Fc region.

In some embodiments, the antigen-binding portion is TCR. The IL-2 moiety may be constructed on a different chain from the TCR. Alternatively, the IL-2 moiety may be on the same heavy chain with one chain (alpha or beta chain) of the TCR. Where the IL-2 moiety is on the same chain with alpha or beta chain of the TCR region, the IL-2 moiety can be operably linked to the hinge region and Fc region (optionally via a linker), which in turn is operably linked to the TCR region, i.e. the IL-2 moiety and the TCR are separated by intervening hinge region and Fc region. In some other embodiments, the IL-2 moiety and the TCR are on the same side (usually N terminal) of the Fc region.

Depending on the desired biochemical (e.g. solubility and stability) and pharmacokinetics properties, different construction formats may be applied, for example, the polypeptide complex may comprise more than one IL-2 moiety or more than one antigen-binding moiety.

As depicted in FIG. 1(a), the polypeptide complex may comprise two heavy chains and one light chain, wherein the antigen-binding moiety is a Fab or TCR, and the first heavy chain comprises, from N-terminal to C-terminal: (a) the IL-2 moiety; (b) optionally, a linker; and (c) one chain of the hinge region and the Fc region;
the second heavy chain comprises, from N-terminal to C-terminal: (d) heavy chain of the Fab or TCR; and (e) the other chain of the hinge region and the Fc region, and
the light chain comprises light chain of the Fab or TCR.

As depicted in FIG. 1(b), the polypeptide complex may comprise two chains, wherein the antigen-binding moiety is a VHH, and the first chain comprises, from N-terminal to C-terminal: (a) the IL-2 moiety; (b) optionally, a linker; and (c) one chain of the hinge region and the Fc region;
the second chain comprises, from N-terminal to C-terminal: (d) the VHH; and (e) the other chain of the hinge region and the Fc region.

As depicted in FIG. 1(c), the polypeptide complex may comprise two chains, and each chain comprises, from N-terminal to C-terminal: (a) the IL-2 moiety; (b) optionally, a linker; and (c) one chain of the hinge region and the Fc region.

As depicted in FIG. 1(d), the polypeptide complex may comprise two heavy chains and two light chains, wherein the antigen-binding moiety is a Fab or TCR, and the first chain comprises, from N-terminal to C-terminal: (a) the IL-2 moiety; (b) optionally, a linker; and (c) one chain of the hinge region and the Fc region; (d) heavy chain of the Fab or TCR;
the light chain comprises light chain of the Fab or TCR.

As depicted in FIG. 1(e), the polypeptide complex may comprise two chains, wherein the antigen-binding moiety is a VHH, and the first chain comprises, from N-terminal to C-terminal: (a) the IL-2 moiety; (b) optionally, a linker; and (c) one chain of the hinge region and the Fc region; (d) the VHH.

As depicted in FIG. 1(f), the polypeptide complex may comprise two chains, wherein the antigen-binding moiety is a scFv, and the first chain comprises, from N-terminal to C-terminal: (a) the IL-2 moiety; (b) optionally, a linker; and (c) one chain of the hinge region and the Fc region; (d) the scFv.

As depicted in FIG. 1(g), the polypeptide complex may comprise two chains, wherein the antigen-binding moiety comprises 2 VHHs, and the first chain comprises, from N-terminal to C-terminal: (a) the IL-2 moiety; (b) optionally, a linker; and (c) one chain of the hinge region and the Fc region; and
the second chain comprises, from N-terminal to C-terminal: (d) the 2 VHHs in tandem; (e) optionally, a linker; and (f) the other chain of the hinge region and the Fc region.

The target antigen for the antigen-binding moiety can be selected from checkpoint molecules such as PD-1, PD-L1, PD-L2, CTLA-4, LAG3, TIM-3, A2aR, TIGIT, VISTA, or tumor associated antigens such as HER2, and BCMA, angiogenesis related factors such as VEGF and PDGF, among others. A numerous variety of antibodies against such antigens are already developed and familiar to those skilled in the art.

The antigen-binding moiety may be from or derived from antibodies which are already known, on the market, or developed de novo, such as any of the following antibodies: trastuzumab, pertuzumab, sacituzumab, abciximab, adalimumab, alefacept, alemtuzumab, basiliximab, belimumab, bezlotoxumab, bevacizuman. canakinumab, certolizumab pegol, cetuximab, daclizumab, denosumab, efalizumab, golimumab, gemtuzumab, infliximab, ipilimumab, ixekizumab, natalizumab, nivolumab, olaratumab, omalizumab, ofatumumab, palivizumab, panitumumab, pembrolizumab, rituximab, ranibizumab, tocilizumab, trastuzumab, secukinumab, and ustekinumab. The variable regions (or at least the CDR regions) of the antigen binding moieties may be same as those of the antibodies which are already known or developed de novo. By “derived from” it is meant that the variable regions are same as those in the parent antibody or have at least 80% homology (e.g. at least 85%, 90%, 95% or above) yet still retain the binding ability to the targeted antigen. For example, the variable regions from parental antibodies may be humanized, affinity matured, or glycosylation modified before being constructed into the polypeptide complexes as disclosed herein. The methods for modification of the variable regions, including CDRs and framework regions, are familiar to a person in the art.

### i) E44 Format

The polypeptide complex as disclosed herein may be constructed as an IL-2/Fab fusion proteins comprising a Fab moiety operably linked to one chain of a Fc region and an IL-2 moiety operably linked to the N terminal of other chain of the Fc region (optionally via a linker). A series of such polypeptide complexes in E44 format are provided herein.

In some embodiments, the IL-2 moiety is consisted of wild-type IL-2 protein. Specifically, the polypeptide complex comprises a first heavy chain as set forth in SEQ ID NO: 15, a second heavy chain as set forth in SEQ ID NO: 33, and a light chain as set forth in SEQ ID NO: 34 (W3XX115-T2U0.E44-1.uIgG4V322). In some embodiments, the IL-2 moiety is consisted of the IL-2 variant that differs from WT IL-2 by a C-terminal truncation (e.g. truncated by 1, 2, 3, 4, 5, 6, 7, 8 or 9 amino acids) as described above. Specifically, the polypeptide complex comprises a first heavy chain as set forth in SEQ ID NO: 17, a second heavy chain as set forth in SEQ ID NO: 33, and a light chain as set forth in SEQ ID NO: 34 (corresponding to W3XX115-T2U0.E44-6.uIgG4V322). In some embodiments, the IL-2 moiety is consisted of the IL-2 variant that differs from WT IL-2 by both a C-terminal truncation (e.g. truncated by 4 amino acids) and a substitution at position I129 as described above. Specifically, the polypeptide complex comprises a first heavy chain as set forth in SEQ ID NO: 18, a second heavy chain as set forth in SEQ ID NO: 33, and a light chain as set forth in SEQ ID NO: 34 (corresponding to W3XX115-T2U0.E44-26.uIgG4V322). In some embodiments, the IL-2 moiety is consisted of the IL-2 variant that differs from WT IL-2 by both a C-terminal truncation (e.g. truncated by 4 amino acids) and a substitution at position E110 as described above. Specifically, the polypeptide complex comprises a first heavy chain as set forth in SEQ ID NO: 19, a second heavy chain as set forth in SEQ ID NO: 33, and a light chain as set forth in SEQ ID NO: 34 (corresponding to W3XX115-T2U0.E44-40.uIgG4V322).

In some embodiments, the IL-2 moiety is consisted of the IL-2 variant that differs from WT IL-2 by both a C-terminal truncation (e.g. truncated by 4 amino acids) and a substitution at position I122 as described above. Specifically, the polypeptide complex comprises a first heavy chain as set forth in SEQ ID NO: 20, a second heavy chain as set forth in SEQ ID NO: 33, and a light chain as set forth in SEQ ID NO: 34 (corresponding to W3XX115-T2U0.E44-41.uIgG4V322). In some embodiments, the IL-2 moiety is consisted of the IL-2 variant that differs from WT IL-2 by both a C-terminal truncation (e.g. truncated by 4 amino acids) and a substitution at position 128 as described above. Specifically, the polypeptide complex comprises a first heavy chain as set forth in SEQ ID NO: 21, a second heavy chain as set forth in SEQ ID NO: 33, and a light chain as set forth in SEQ ID NO: 34 (corresponding to W3XX115-T2U0.E44-42.uIgG4V322). In some embodiments, the IL-2 moiety is consisted of the IL-2 variant that differs from WT IL-2 by both a C-terminal truncation (e.g. truncated by 4 amino acids) and a substitution at position K32 as described above. Specifically, the polypeptide complex comprises a first heavy chain as set forth in SEQ ID NO: 22, a second heavy chain as set forth in SEQ ID NO: 33, and a light chain as set forth in SEQ ID NO: 34 (corresponding to W3XX115-T2U0.E44-43.uIgG4V322). In some embodiments, the IL-2 moiety is consisted of the IL-2 variant that differs from WT IL-2 by both a C-terminal truncation (e.g. truncated by 4 amino acids) and a substitution at position E52 as described above. Specifically, the polypeptide complex comprises a first heavy chain as set forth in SEQ ID NO: 23, a second heavy chain as set forth in SEQ ID NO: 33, and a light chain as set forth in SEQ ID NO: 34 (corresponding to W3XX115-T2U0.E44-44.uIgG4V322). In some embodiments, the IL-2 moiety is consisted of the IL-2 variant that differs from WT IL-2 by both a C-terminal truncation (e.g. truncated by 4 amino acids) and a substitution at position K76 as described above. Specifically, the polypeptide complex comprises a first heavy chain as set forth in SEQ ID NO: 24, a second heavy chain as set forth in SEQ ID NO: 33, and a light chain as set forth in SEQ ID NO: 34 (corresponding to W3XX115-T2U0.E44-45.uIgG4V322). In some embodiments, the IL-2 moiety is consisted of the IL-2 variant that differs from WT IL-2 by both a C-terminal truncation (e.g. truncated by 4 amino acids) and a substitution at position F78 as described above. Specifically, the polypeptide complex comprises a first heavy chain as set forth in SEQ ID NO: 25, a second heavy chain as set forth in SEQ ID NO: 33, and a light chain as set forth in SEQ ID NO: 34 (corresponding to W3XX115-T2U0.E44-46.uIgG4V322).

In some embodiments, the IL-2 moiety is consisted of the IL-2 variant that differs from WT IL-2 by both a C-terminal truncation (e.g. truncated by 4 amino acids) and a substitution at position P82 as described above. Specifically, the polypeptide complex comprises a first heavy chain as set forth in SEQ ID NO: 26, a second heavy chain as set forth in SEQ ID NO: 33, and a light chain as set forth in SEQ ID NO: 34 (corresponding to W3XX115-T2U0.E44-47.uIgG4V322). In some embodiments, the IL-2 moiety is consisted of the IL-2 variant that differs from WT IL-2 by both a C-terminal truncation (e.g. truncated by 4 amino acids) and substitutions at position E110 and I129 as described above. Specifically, the polypeptide complex comprises a first heavy chain as set forth in SEQ ID NO: 27, a second heavy chain as set forth in SEQ ID NO: 33, and a light chain as set forth in SEQ ID NO: 34 (corresponding to W3XX115-T2U0.E44-48.uIgG4V322). In some embodiments, the IL-2 moiety is consisted of the IL-2 variant that differs from WT IL-2 by both a C-terminal truncation (e.g. truncated by 4 amino acids) and substitutions at position K32 and I129 as described above. Specifically, the polypeptide complex comprises a first heavy chain as set forth in SEQ ID NO: 28, a second heavy chain as set forth in SEQ ID NO: 33, and a light chain as set forth in SEQ ID NO: 34 (corresponding to W3XX115-T2U0.E44-49.uIgG4V322).

In some embodiments, the IL-2 moiety is consisted of the IL-2 variant that differs from WT IL-2 by both a C-terminal truncation (e.g. truncated by 4 amino acids) as described above. Specifically, the polypeptide complex comprises a first heavy chain as set forth in SEQ ID NO: 52, a second heavy chain as set forth in SEQ ID NO: 57, and a light chain as set forth in SEQ ID NO: 58 (corresponding to W3XX115-T2U3.E44-6.uIgG4V322). In some embodiments, the IL-2 moiety is consisted of the IL-2 variant that differs from WT IL-2 by both a C-terminal truncation (e.g. truncated by 4 amino acids) and substitutions at position I129 as described above. Specifically, the polypeptide complex comprises a first heavy chain as set forth in SEQ ID NO: 53, a second heavy chain as set forth in SEQ ID NO: 57, and a light chain as set forth in SEQ ID NO: 58 (corresponding to W3XX115-T2U3.E44-26.uIgG4V322). In some embodiments,

In some embodiments, the polypeptide the IL-2 moiety is consisted of the IL-2 variant that differs from WT IL-2 by both a C-terminal truncation (e.g. truncated by 4 amino acids) and substitutions at position I129 as described above. Specifically, the polypeptide complex comprises a first heavy chain as set forth in SEQ ID NO: 54, a second heavy chain as set forth in SEQ ID NO: 57, and a light chain as set forth in SEQ ID NO: 58 (corresponding to W3XX115-T2U3.E44-26.uIgG4V322). In some embodiments, the IL-2 moiety is consisted of the IL-2 variant that differs from WT IL-2 by both a C-terminal truncation (e.g. truncated by 4 amino acids) and substitutions at position R38W as described above. Specifically, the polypeptide complex comprises a first heavy chain as set forth in SEQ ID NO: 55, a second heavy chain as set forth in SEQ ID NO: 57, and a light chain as set forth in SEQ ID NO: 58 (corresponding to W3XX115-T2U3.E44-20.uIgG4V322). In some embodiments, the IL-2 moiety is consisted of the IL-2 variant that differs from WT IL-2 by both a C-terminal truncation (e.g. truncated by 4 amino acids) and substitutions at position F42I and I129 as described above. Specifically, the polypeptide complex comprises a first heavy chain as set forth in SEQ ID NO: 56, a second heavy chain as set forth in SEQ ID NO: 57, and a light chain as set forth in SEQ ID NO: 58 (corresponding to W3XX115-T2U3.E44-33.uIgG4V322).

### ii) Z20 Format

The polypeptide complex as disclosed herein may be constructed as a IL-2/Fc fusion proteins comprising a IL-2 moiety operably linked to the N terminal of each chain of the Fc region (optionally via a linker). In some embodiments, the IL-2 moiety is consisted of the IL-2 variant that differs from WT IL-2 by a C-terminal truncation (e.g. truncated by 1, 2, 3, 4, 5, 6, 7, 8 or 9 amino acids) as described above. Specifically, the polypeptide complex comprises a heavy chain as set forth in SEQ ID NOs: 29 (corresponding to W3XX115-T2.Z20-1.uIgG4V322).

In some embodiments, the IL-2 moiety is consisted of the IL-2 variant that differs from WT IL-2 by both a C-terminal truncation (e.g. truncated by 4 amino acids) and a substitution at position I129 as described above. Specifically, the polypeptide complex comprises a first heavy chain as set forth in SEQ ID NO: 30 (corresponding to W3XX115-T2.Z20-2.uIgG4V322).

In some embodiments, the IL-2 moiety is consisted of the IL-2 variant that differs from WT IL-2 by both a C-terminal truncation (e.g. truncated by 4 amino acids), a substitution at position I129 and a substitution at position E110 as described above. Specifically, the polypeptide complex comprises a first heavy chain as set forth in SEQ ID NO: 31 (corresponding to W3XX115-T2.Z20-4.uIgG4V322).

In some embodiments, the IL-2 moiety is consisted of the IL-2 variant that differs from WT IL-2 by both a C-terminal truncation (e.g. truncated by 4 amino acids), a substitution at position I129 and a substitution at position K32 as described above. Specifically, the polypeptide complex comprises a first heavy chain as set forth in SEQ ID NO: 32 (corresponding to W3XX115-T2.Z20-5.uIgG4V322).

iii) Z73 Format

The polypeptide complex as disclosed herein may be constructed as IL-2/VHH fusion proteins comprising two VHHs in tandem operably linked to one chain of a Fc region and a IL-2 moiety operably linked to the N terminal of other chain of the Fc region (optionally via a linker). A series of such polypeptide complexes in Z73 format are provided herein.

In some embodiments, the IL-2 moiety is consisted of the IL-2 variant that differs from WT IL-2 by both a C-terminal truncation (e.g. truncated by 4 amino acids) and substitutions at position F42I, E110R and I129 as described above. Specifically, the polypeptide complex comprises a first chain as set forth in SEQ ID NO: 59, a second chain as set forth in SEQ ID NO: 75 (corresponding to W3XX115-T2U10.Z73-50.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D and I129 as described above. Specifically, the polypeptide complex comprises a first chain as set forth in SEQ ID NO: 60, a second chain as set forth in SEQ ID NO: 75 (corresponding to W3XX115-T2U10.Z73-51.uIgG4V322).

In some embodiments, the IL-2 moiety is consisted of the IL-2 variant that differs from WT IL-2 by substitutions at position L18R, Q22E, F42I and Q126K as described above. Specifically, the polypeptide complex comprises a first chain as set forth in SEQ ID NO: 61, a second chain as set forth in SEQ ID NO: 75 (corresponding to W3XX115-T2U10.Z73-52.uIgG4V322).

In some embodiments, the IL-2 moiety is consisted of the IL-2 variant that differs from WT IL-2 by substitutions at position L19H, F42I, C125I and Q126E as described above. Specifically, the polypeptide complex comprises a first chain as set forth in SEQ ID NO: 62, a second chain as set forth in SEQ ID NO: 75 (corresponding to W3XX115-T2U10.Z73-53.uIgG4V322).

In some embodiments, the IL-2 moiety is consisted of the IL-2 variant that differs from WT IL-2 by substitutions at position T3A, D20N, F42I, N71K and Q125S as described above. Specifically, the polypeptide complex comprises a first chain as set forth in SEQ ID NO: 63, a second chain as set forth in SEQ ID NO: 75 (corresponding to W3XX115-T2U10.Z73-54.uIgG4V322).

In some embodiments, the IL-2 moiety is consisted of the IL-2 variant that differs from WT IL-2 by both a C-terminal truncation (e.g. truncated by 4 amino acids) and substitutions at position Q13D, F42I, K32D and I129 as described above. Specifically, the polypeptide complex comprises a first chain as set forth in SEQ ID NO: 64, a second chain as set forth in SEQ ID NO: 75 (corresponding to W3XX115-T2U10.Z73-55.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, D84K and I129 as described above. Specifically, the polypeptide complex comprises a first chain as set forth in SEQ ID NO: 65, a second chain as set forth in SEQ ID NO: 75 (corresponding to W3XX115-T2U10.Z73-56.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, S87R and I129 as described above. Specifically, the polypeptide complex comprises a first chain as set forth in SEQ ID NO: 66, a second chain as set forth in SEQ ID NO: 75 (corresponding to W3XX115-T2U10.Z73-57.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, N88K and I129 as described above. Specifically, the polypeptide complex comprises a first chain as set forth in SEQ ID NO: 67, a second chain as set forth in SEQ ID NO: 75 (corresponding to W3XX115-T2U10.Z73-58.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, V91E and I129 as described above. Specifically, the polypeptide complex comprises a first chain as set forth in SEQ ID NO: 68, a second chain as set forth in SEQ ID NO: 75 (corresponding to W3XX115-T2U10.Z73-59.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, I92R and I129 as described above. Specifically, the polypeptide complex comprises a first chain as set forth in SEQ ID NO: 69, a second chain as set forth in SEQ ID NO: 75 (corresponding to W3XX115-T2U10.Z73-60.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, I92D and I129 as described above. Specifically, the polypeptide complex comprises a first chain as set forth in SEQ ID NO: 70, a second chain as set forth in SEQ ID NO: 75 (corresponding to W3XX115-T2U10.Z73-61.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, L94D and I129 as described above. Specifically, the polypeptide complex comprises a first chain as set forth in SEQ ID NO: 71, a second chain as set forth in SEQ ID NO: 75 (corresponding to W3XX115-T2U10.Z73-62.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, E95R and I129 as described above. Specifically, the polypeptide complex comprises a first chain as set forth in SEQ ID NO: 72, a second chain as set forth in SEQ ID NO: 75 (corresponding to W3XX115-T2U10.Z73-63.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, N119R and I129 as described above. Specifically, the polypeptide complex comprises a first chain as set forth in SEQ ID NO: 73, a second chain as set forth in SEQ ID NO: 75 (corresponding to W3XX115-T2U10.Z73-64.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, T123K and I129 as described above. Specifically, the polypeptide complex comprises a first chain as set forth in SEQ ID NO: 74, a second chain as set forth in SEQ ID NO: 75 (corresponding to W3XX115-T2U10.Z73-65.uIgG4V322).

In some embodiments, the IL-2 moiety is consisted of the IL-2 variant that differs from WT IL-2 by substitutions at position L18R, Q22E and Q126K as described above. Specifically, the polypeptide complex comprises a first chain as set forth in SEQ ID NO: 76, a second chain as set forth in SEQ ID NO: 75 (corresponding to W3XX115-T2U10.Z73-66.uIgG4V322).

In some embodiments, the IL-2 moiety is consisted of the IL-2 variant that differs from WT IL-2 by substitutions at position L19H, C125I and Q126E as described above. Specifically, the polypeptide complex comprises a first chain as set forth in SEQ ID NO: 77, a second chain as set forth in SEQ ID NO: 75 (corresponding to W3XX115-T2U10.Z73-67.uIgG4V322).

In some embodiments, the IL-2 moiety is consisted of the IL-2 variant that differs from WT IL-2 by substitutions at position T3A, D20N, N71K and Q125S as described above. Specifically, the polypeptide complex comprises a first chain as set forth in SEQ ID NO: 78, a second chain as set forth in SEQ ID NO: 75 (corresponding to W3XX115-T2U10.Z73-68.uIgG4V322).

In some embodiments, the IL-2 moiety is consisted of the IL-2 variant that differs from WT IL-2 by both a C-terminal truncation (e.g. truncated by 4 amino acids) and substitutions at position Q13R, F42I, K32D and I129 as described above. Specifically, the polypeptide complex comprises a first chain as set forth in SEQ ID NO: 104, a second chain as set forth in SEQ ID NO: 75 (corresponding to W3XX115-T2U10.Z73-69.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, D84T and I129 as described above. Specifically, the polypeptide complex comprises a first chain as set forth in SEQ ID NO: 105, a second chain as set forth in SEQ ID NO: 75 (corresponding to W3XX115-T2U10.Z73-70.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, S87I and I129 as described above. Specifically, the polypeptide complex comprises a first chain as set forth in SEQ ID NO: 106, a second chain as set forth in SEQ ID NO: 75 (corresponding to W3XX115-T2U10.Z73-71.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, V91S and I129 as described above. Specifically, the polypeptide complex comprises a first chain as set forth in SEQ ID NO: 107, a second chain as set forth in SEQ ID NO: 75 (corresponding to W3XX115-T2U10.Z73-72.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, E95Y and I129 as described above. Specifically, the polypeptide complex comprises a first chain as set forth in SEQ ID NO: 108, a second chain as set forth in SEQ ID NO: 75 (corresponding to W3XX115-T2U10.Z73-73.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, N119Q and I129 as described above. Specifically, the polypeptide complex comprises a first chain as set forth in SEQ ID NO: 109, a second chain as set forth in SEQ ID NO: 75 (corresponding to W3XX115-T2U10.Z73-74.uIgG4V322).

In some embodiments, the IL-2 moiety is consisted of the IL-2 variant that differs from WT IL-2 by both a C-terminal truncation (e.g. truncated by 4 amino acids) and substitutions at position F42I, K32D, T123V and I129 as described above. Specifically, the polypeptide complex comprises a first chain as set forth in SEQ ID NO: 110, a second chain as set forth in SEQ ID NO: 75 (corresponding to W3XX115-T2U10.Z73-75.uIgG4V322).

### Fc Domain

The disclosure provides Fc-fusion proteins and polypeptide complexes comprising a Fc region and the human IL-2 variant as described above. The Fc domain may be a wild-type Fc or an Fc variant. A wild-type Fc may be a human IgG1, IgG2, IgG3 or IgG4 Fc. In some embodiments, the wild-type Fc is a human IgG1 Fc. The Fc variant comprises one or more amino acid residue modifications (e.g. substitutions, insertions and/or deletions) compared to the wild-type Fc (e.g, human IgG1, IgG2, IgG3 or IgG4 Fc). In some embodiments, the Fc variant comprises one or more amino acid residue modifications (e.g. substitutions, insertions and/or deletions) compared to wild-type human IgG1 Fc. In some embodiments, the Fc variant comprises one or more amino acid residue modifications (e.g. substitutions, insertions and/or deletions) compared to wild-type human IgG4 Fc.

There are many known mutations that exist for increasing or reducing ADCC, ADCP and CDC. One example of human IgG1 heavy chain mutations is LALA mutation, which prevents all effector function, i.e., essentially functions to ADCC, ADCP and CDC. The hIgGI LALA sequence includes two mutations, L234A and L235A (EU numbering), which suppress FcgR binding. The “EU numbering system” or “EU index” is generally used when referring to a residue in an immunoglobulin heavy chain constant region (e.g., the EU index reported in Kabat et al. Sequences of Proteins of Immunological Interest (5^{th} Ed.), US Dept. of Health and Human Services, PHS, NIH, NIH Publication no. 91-3242). The “EU numbering as in Kabat” or “EU index as in Kabat” refers to the residue numbering of the human IgG1 EU antibody. Unless stated otherwise herein, references to residue numbers in the constant domain of Fc regions means residue numbering by the EU numbering system.

Said one or more amino acid modifications comprised in the Fc variant may alter the binding to one or more FcγR receptors, alter the binding to FcRn receptors, etc. In certain embodiments, the Fc domain of the fusion proteins comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6 (1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70:3269-3277 (2010); and Hinton, P. et al, J. Immunology, 176:346-356 (2006).

The two chains of the Fc domain may associate together via a disulfide bond. In some embodiments, the Fc domain comprises one or more amino acid modifications (e.g. substitutions) in the interface of the Fc region to facilitate and/or promote heterodimerization. For example, the two chains of the Fc domain are engineered to comprise a “knob-into-hole” structure to promote heterodimerization, which includes introduction of a protuberance (“knob”) into a first Fc polypeptide and a cavity (“hole”) into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168. Specifically, the Fc domain may comprise at least one “knob” (protuberance) and at least one “hole” (cavity), wherein presence of the “knob” and “hole” enhances formation of a complex or heterodimer (for more detail see WO 2005/063816). In some embodiments, the Fc domain as disclosed herein comprises a first and a second Fc polypeptide chain, wherein the first and second polypeptide each comprises one or more mutations with respect to wild type human IgG1 Fc. The IL-2 domain may be fused to one chain of the Fc domain comprising a “knob” mutation while the V_{H} region of the antigen-binding portion is fused to the other chain comprising a “hole” mutation, or vice versa. In at least one embodiment, a “hole” mutation is Y349C, T366S, L368A, and/or Y407V, and a “knob” mutation is S354C and/or T366W.

In certain embodiments, the Fc domain of the fusion proteins comprise one or more amino acid substitution(s) that alters the antibody-dependent cellular cytotoxicity (ADCC) and/or complement dependent cytotoxicity (CDC). Certain amino acid residues at CH2 domain of the Fc region can be substituted to provide for reduced ADCC activity.

In some embodiments, the Fc domain is a IgG4 Fc variant that comprises a “FALA” mutation (i.e. F234A/L235A), which reduces the binding with Fc receptors or complement receptors. In some other embodiments, the Fc domain is a IgG4 Fc variant with truncated hinge region. In still some other embodiments, the Fc domain is a IgG4 Fc variant that comprises a S228P mutation, which may reduce IgG4 Fab-arm exchange.

In some specific embodiments, the Fc variant comprises two chains, wherein the amino acid sequence of the first chain has at least 80%, e.g 80%, 85%, 90%, 95% or more (e.g. 100%) sequence identity to wild-type human IgG1, IgG2, IgG4, or IgG4 with hinge truncation.

### Properties of the Polypeptide Complexes

The present disclosure provides affinity attenuated and potency reduced IL-2 variants and polypeptide complexes comprising the IL-2 variants. The IL-2 variants have the potential of regulated potency/toxicity, PK and PD, thus may serve as a novel immunotherapy agent with improved anti-tumor efficacy and autoimmune disease therapy.

The functionality of these IL-2 variants and IL-2 comprising polypeptide complexes may be assessed by in vitro or in vivo assays.

In some embodiments, the affinity of the IL-2 comprising polypeptide complexes to IL-2Rα or IL-2ß is determined in BIAcore binding assays.

Alternatively, the binding affinity of the IL-2 comprising polypeptide complexes is determined by FACS, using cell lines that express IL-2Rβ (intermediate affinity receptor) and/or IL-2Rα (high affinity receptor), such as HH cells, NK92 and MJ20 cells.

In some embodiments, the effect of the IL-2 variants and polypeptide complexes is evaluated by quantifying a signaling pathway measured by phosphorylation of certain factors, such as STAT5 phosphorylation. STAT5 plays an important role in the maintenance of normal immune function and homeostasis, both of which are regulated by specific members of IL-2 family of cytokines.

Regulatory T cells (Treg) play a critical role to maintain immune homeostasis and self-tolerance, and is crucial in controlling the development of allergies and autoimmune diseases. Activated CD8+ T cells are very important for immune defense against tumor. IL-2 is able to induce proliferation of Treg cells and activated CD8+ T by signaling through high affinity IL-2 receptor. In some embodiments, the effect of the IL-2 variants and polypeptide complexes is evaluated by assessing T cell activation, such as CD8+ T cell or Treg cell activation.

The IL-2 comprising polypeptide complexes of the present disclosure provide at least one of the following properties:
(a) attenuated binding affinity to at least one of IL-2Rα, IL-2Rβ/γc, and the combined IL-2Rα/B/γc complex;
(b) more moderate potency in simulating immune cell proliferation, such as CD8+ T cell and NK cell proliferation;
(c) reduced but retained potency in activating immune cells such as activated CD8+ T cells or Treg cells; and
(d) enhanced thermal stability, serum stability and in vivo PK;
(e) no obvious toxicity in vivo;
(f) prominent anti-tumor efficacy in mouse models.

### Nucleic Acid Molecules Encoding the IL-2 Variants

In some aspects, the disclosure is directed to an isolated nucleic acid molecule, comprising a nucleic acid sequence encoding the IL-2 variant or the Fc fusion protein as disclosed herein.

Nucleic acids of the disclosure can be obtained using standard molecular biology techniques. The isolated nucleic acid encoding the IL-2 variant can be operatively linked to another DNA molecule encoding an Fc domain. Similarly, a nucleic acid encoding the antigen-binding portion can be operatively linked to another DNA molecule encoding an Fc domain. DNA fragments encompassing these regions can be obtained by standard PCR amplification.

Once DNA fragments encoding IL-2 moiety, the antigen-binding portion and Fc domains (or constant regions) are obtained, these DNA fragments can be further manipulated by standard recombinant DNA techniques, for example incorporated into expression vectors as is known in the art. In some embodiments, nucleic acids encoding these DNA fragments are each contained within a single expression vector, generally under different or the same promoter control. In some other embodiments, nucleic acids encoding these DNA fragments are operably linked and contained in a single expression vector under the control of the same promoter. The term “operatively linked”, as used in this context, is intended to mean that the two DNA fragments are joined such that the amino acid sequences encoded by the two DNA fragments remain in-frame.

### Host Cells

Host cells as disclosed in the present disclosure may be any cell which is suitable for expressing the fusion proteins of the present disclosure, for instance, bacterial cells, yeast, mammalian cells. Mammalian host cells for expressing the fusion proteins of the present disclosure include Chinese Hamster Ovary (CHO cells) (including dhfr CHO cells, described in Urlaub and Chasin, (1980) Proc. Natl. Acad. ScL USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular, for use with NSO myeloma cells, another expression system is the GS gene expression system disclosed in WO 87/04462, WO 89/01036 and EP 338,841. When recombinant expression vectors encoding the antibody are introduced into mammalian host cells, the fusion proteins are produced by culturing the host cells for a period of time sufficient to allow for expression of the fusion protein in the host cells or, secretion of the fusion protein into the culture medium in which the host cells are grown. The fusion proteins can be recovered from the culture medium using standard protein purification methods.

### Pharmaceutical Compositions

In some aspects, the disclosure is directed to a pharmaceutical composition comprising the fusion protein or polypeptide complex as disclosed herein and a pharmaceutically acceptable carrier. In some aspects, the disclosure is directed to a pharmaceutical composition comprising a nucleic acid molecule encoding the fusion protein or polypeptide complex as disclosed herein and a pharmaceutically acceptable carrier.

### Components of the Compositions

The pharmaceutical composition may optionally contain one or more additional pharmaceutically active ingredients, such as an antibody. The pharmaceutical compositions of the disclosure also can be administered in a combination therapy with, for example, another immune-stimulatory agent, anti-cancer agent, an antiviral agent, or a vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, the composition may include one or more anti-oxidants such as methionine, reducing antibody or antigen binding fragment thereof that may be oxidized. The oxidation reduction may prevent or reduce a decrease in binding affinity, thereby enhancing protein stability and extended shelf life. Thus, in some embodiments, the present disclosure provides a composition comprising fusion proteins and one or more anti-oxidants such as methionine. The present disclosure further provides a variety of methods, wherein a fusion protein is mixed with one or more anti-oxidants, such as methionine, so that the fusion protein can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the polypeptide complexes of the disclosure may be administered in various ranges. These include about 100 μg/kg body weight to about 10 mg/kg body weight per dose; about 100 μg/kg body weight to about 1 mg/kg body weight per dose; about 1 μg/kg body weight to about 10 mg/kg body weight per dose. Other ranges include about 100 μg/kg body weight to about 200 μg/kg body weight per dose; about 200 μg/kg body weight to about 300 μg/kg body weight per dose; about 300 μg/kg body weight to about 400 μg/kg body weight per dose; about 400 μg/kg body weight to about 0.5 μg/kg body weight per dose; and about 0.5 mg/kg body weight to about 1 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the polypeptide complexes of the disclosure are preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the polypeptide complexes of the present disclosure will comprise multiple doses of the selected drug product over a period of weeks or months. More specifically, the polypeptide complexes of the present disclosure may be administered once every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

### Applications of the Disclosure

The polypeptide complexes, pharmaceutical compositions and methods of the present disclosure have numerous in vitro and in vivo utilities involving, for example, enhancement of immune response. For example, these molecules can be administered to cells in culture, in vitro or ex vivo, or to human subjects, e.g., in vivo, to enhance immunity in a variety of situations. The immune response can be modulated, for instance, augmented, stimulated or up-regulated.

For instance, the subjects include human patients in need of enhancement of an immune response. The methods are particularly suitable for treating human patients having a disorder that can be treated by augmenting an immune response (e.g., the NK/T-cell mediated immune response). In a particular embodiment, the methods are particularly suitable for treatment of cancer in vivo. To achieve enhancement of immunity, the polypeptide complexes can be administered alone or in combination with another therapy. When polypeptide complexes are administered together with another agent, the two can be administered in either order or simultaneously.

For instance, the subjects include human patients in need of suppression of an immune response. The methods are particularly suitable for treating human patients having a disorder that can be treated by suppressing an immune response (e.g., the Treg mediated immune suppression). In a particular embodiment, the methods are particularly suitable for treatment of autoimmune disease in vivo. To achieve suppression of immunity, the polypeptide complexes can be administered alone or in combination with another therapy. When polypeptide complexes are administered together with another agent, the two can be administered in either order or simultaneously.

### Treatment of Disorders Including Cancers

In some aspects, the present disclosure provides a method of treating a disorder or a disease in a mammal, which comprises administering to the subject (for example, a human) in need of treatment a therapeutically effective amount of the polypeptide complexes as disclosed herein. In some aspects, the present disclosure provides a method of treating a disorder or a disease in a mammal, which comprises administering to the subject (for example, a human) in need of treatment a therapeutically effective amount of a nucleic acid molecule encoding the polypeptide complexes as disclosed herein. The disorder or disease may be a cancer.

A variety of cancers, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer.

In some embodiments, examples of cancer include but not limited to B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In other embodiments, the disorder or disease may be autoimmune and inflammatory diseases, including but not limited to, type 1 diabetes, multiple sclerosis, lupus, rheumatoid arthritis, Systemic Lupus Erythematosus, autoimmune hepatitis, Antiphospholipid Syndrome, Wegener's Granulomatosis, Bullous Pemphigoid, Churg Strauss Syndrome, Thyroid diseases, including Graves' disease, Inflammatory bowel disease, Guillain-Barre syndrome, Psoriasis, Myasthenia gravis and Vasculitis.

Stimulation/Suppression of an Immune Response without Incurring Cytotoxicity

In some aspects, the disclosure also provides a method of enhancing (for example, stimulating) or suppressing an immune response in a subject comprising administering a polypeptide complex of the disclosure to the subject such that an immune response in the subject is enhanced while no undesired side effects are presented. For example, the subject is a mammal. In a specific embodiment, the subject is a human.

The term “enhancing an immune response” or its grammatical variations, means stimulating, evoking, increasing, improving, or augmenting any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of enhancement of immune response include increased CD4+T, especially helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example IL-15 production or IFN-γ production), regression of tumors, survival of tumor bearing animals, antibody production, immune cell proliferation, expression of cell surface markers, and cytotoxicity. Typically, methods of the disclosure enhance the immune response by a mammal when compared to the immune response by an untreated mammal or a mammal not treated using the methods as disclosed herein. In one embodiment, the immune response is cytokine production, particularly IFN-γ production or IL-12 production. In another embodiment, the immune response is enhanced B cell proliferation. In contrary, “suppressing an immune response” means reducing, alleviating, decreasing or lowering the response of a mammal's immune system, which is often desired in autoimmune diseases where the immune system is overactive. The polypeptide complexes as disclosed herein may be used alone as a monotherapy, or more often, used in combination with cell immunotherapies, targeted therapies, chemical therapies or radiotherapies.

### Application in Cellular Immunotherapies and Gene Therapy

In some embodiments, IL-2 fusion proteins and variants as disclosed herein are used in cellular immunotherapy with IL-2 variants secretion, the IL-2 variants gene are constructed into therapeutic engineered immune cells, including but not limited to Tumor-Infiltrating Lymphocyte (TIL), Engineered T Cell Receptor (TCR-T), Chimeric Antigen Receptor (CAR) T Cell, CAR NK Cell, CAR Macrophage Cell, Natural Killer (NK) Cell.

In some embodiments, IL-2 fusion proteins and variants as disclosed herein are used in gene therapy. The gene coding IL-2 variants are integrated into therapeutic vectors, such as lentivirus, AAV, poxvirus, herpes zoster virus, oncolytic virus and other RNA/DNA vectors.

Combined Use with Cellular Immunotherapies

In some embodiments, the IL-2 fusion proteins as disclosed herein are used in combination with a cellular immunotherapy, also known as adoptive cell therapy. As is generally known, cellular immunotherapy is a form of treatment that uses the cells of human body's immune system to eliminate cancer. Some of these approaches involve directly isolating our own immune cells and simply expanding their numbers (e.g. performed by activating and expanding the immune cells of patient outside of the body and infused into the patient), whereas others involve genetically engineering immune cells (via gene therapy) to enhance their cancer-fighting capabilities. Cellular immunotherapies can be deployed in different ways, including but not limited to Tumor-Infiltrating Lymphocyte (TIL) therapy, Engineered T Cell Receptor (TCR-T) therapy, Chimeric Antigen Receptor (CAR) T Cell therapy, CAR NK Cell therapy, CAR Macrophage Cell therapy, Natural Killer (NK) Cell therapy.

Combined Use with Gene Therapies

In some embodiments, the IL-2 fusion proteins as disclosed herein are used in combination with a gene therapy. The gene coding IL-2 variants may be delivered to a subject by therapeutic vectors, such as viruses, including lentivirus, AAV, poxvirus, herpes zoster virus, oncolytic virus. Transfer of gene may be performed through transformation where under specific conditions the gene is directly taken up by the bacterial cells, transduction where a bacteriophage is used to transfer the genetic material and lastly transfection that involves forceful delivery of gene using either viral or non-viral vectors. The non-viral transfection methods are subdivided into physical, chemical and biological. The physical methods include electroporation, biolistic, microinjection, laser, elevated temperature, ultrasound and hydrodynamic gene transfer. The chemical methods utilize calcium-phosphate, DAE-dextran, liposomes and nanoparticles for transfection. The biological methods are increasingly using viruses for gene transfer, these viruses could either integrate within the genome of the host cell conferring a stable gene expression, whereas few other non-integrating viruses are episomal and their expression is diluted proportional to the cell division.

Combined Use with Targeted Therapies and Chemotherapies

The heterodimeric and homodimeric fusion proteins as disclosed herein may be administered as the sole active ingredient or in conjunction with, e.g. as an adjuvant to or in combination to, other drugs e.g. anti-cancer agents, immunomodulating agents or other anti-inflammatory agents, e.g. for the treatment or prevention of diseases mentioned above.

The term “anti-cancer agent” or “anti-proliferative agent” means any agent that can be used to treat a cell proliferative disorder such as cancer, and includes, but is not limited to, therapeutic antibodies, cytotoxic agents, cytostatic agents, anti-angiogenic agents, debulking agents, chemotherapeutic agents, radiotherapy and radiotherapeutic agents, targeted anti-cancer agents, BRMs, cancer vaccines, cytokines, hormone therapies, radiation therapy and anti-metastatic agents and immunotherapeutic agents.

For example, the fusion proteins as described herein may be used in combination with a wide variety of monoclonal antibodies, such as antibodies against tumor related antigens, stroma related antigens or pathways, e.g. PD-1/PD-L1, TIM-3, LAG-3, VEGF, HER2, CTLA-4; antibodies against leukocyte receptors, e.g., MHC, CD2, CD3, CD4, CD7, CD8, CD25, CD28, CD40, CD45, CD58, CD80, CD86 or their ligands; CD3 engager antibodies, NK engager antibodies; ADCC enabling anti-Tumor associated antigens; monoclonal antibodies to TNF, among others. The antibodies may include but not limited to, abciximab, adalimumab, alefacept, alemtuzumab, basiliximab, belimumab, bezlotoxumab, canakinumab, certolizumab pegol, cetuximab, daclizumab, denosumab, efalizumab, golimumab, inflectra, ipilimumab, ixekizumab, natalizumab, nivolumab, olaratumab, omalizumab, palivizumab, panitumumab, pembrolizumab, rituximab, tocilizumab, trastuzumab, secukinumab, and ustekinumab.

The antibodies may be monospecific or multi-specific. For example, the antibodies may be trispecific antibodies (TrAbs or TrioMabs), which have two variable segments for antigen binding and an Fc component to recruit immune cells. One example of TrAb is Catumaxomab for treating EpCam positive gastric and ovarian tumors. The antibodies may also be bispecific T cell engager antibodies (BiTE), such as Blinatumomab, MEHD7945A, ABT-122, XmAb5871 etc.

In some embodiments, the heterodimeric and homodimeric fusion proteins as described herein may be used in combination with immunomodulatory compounds, e.g. a recombinant binding molecule having at least a portion of the extracellular domain of CTLA4 or a mutant thereof; adhesion molecule inhibitors, e.g. LFA-1 antagonists, ICAM-1 or -3 antagonists, VCAM-4 antagonists or VLA-4 antagonists; blockers of proinflammatory cytokines, IL-1 blockers; chemokines blockers; or a chemotherapeutic agent.

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI. Examples of anti-cancer agents that may be used in combination with the site-specific constructs of the present disclosure (either as a component of a site specific conjugate or in an unconjugated state) include, but are not limited to, e.g. paclitaxel, gemcitabine, cisplatinum, doxorubicin, 5-fluorouracil, capecitabine, combretastatin, leucovorin etc.

For example, the heterodimeric and homodimeric fusion proteins as described herein may be used in combination with DMARD, e.g. Gold salts, sulphasalazine, antimalarias, methotrexate, D-penicillamine, azathioprine, mycophenolic acid, cyclosporine A, tacrolimus, sirolimus, minocycline, lefiunomide, glococorticoids; a calcineurin inhibitor, e.g. cyclosporin A or FK 506; a modulator of lymphocyte recirculation, e.g. FTY720 and FTY720 analogs; a mTOR inhibitor, e.g. rapamycin, 40-O-(2-hydroxyethyl)-rapamycin, CCI779, ABT578, AP23573 or TAFA-93; an ascomycin having immunosuppressive properties, e.g. ABT-281, ASM981, etc.; corticosteroids; cyclo-phosphamide; azathioprene; methotrexate; lefiunomide; mizoribine; mycophenolic acid; myco-phenolate mofetil; 15-deoxyspergualine or an immunosuppressive homologue, analogue or derivative thereof; immunosuppressive

It will be appreciated that, in selected embodiments as discussed above, such anti-cancer agents may comprise conjugates and may be associated with the disclosed heterodimeric and homodimeric polypeptide complexes prior to administration. More specifically, in certain embodiments selected anti-cancer agents will be linked to the unpaired cysteines of the engineered polypeptide complexes to provide engineered conjugates as set forth herein. Accordingly, such engineered conjugates are expressly contemplated as being within the scope of the present disclosure. In other embodiments, the disclosed anti-cancer agents will be given in combination with site-specific conjugates comprising a different therapeutic agent as set forth above.

It will be easily appreciated that, the anti-cancer agents or immunomodulating agents to be used in combination with the heterodimeric and homodimeric polypeptide complexes as disclosed herein should be compatible with the polypeptide complexes, i.e. would not reduce, disturb, or eliminate the effect of the polypeptide complexes as disclosed herein, and preferably provide a coordinating or even synergistic effect.

### As an Immune Enhancing Component/Moiety in a Multi-Specific Antibody

The polypeptide complexes as disclosed herein may be associated with a second antigen-specific binding portion to form a multi-specific antibody complex. For example, an antigen-binding portion (e.g. comprising a heavy chain variable region and a light chain variable region) may be fused with the N terminal or C terminal of the IL-2 moiety. Such multi-specific antibody complex not only have a high affinity to a targeted antigen, but also the potency of IL-2 in promoting immune cell activation.

Combined Use with Radiotherapies

The present disclosure also provides for the combination of the heterodimeric and homodimeric polypeptide complexes thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

As an Immune Suppressing without Incurring Cytotoxicity

In some aspects, the disclosure also provides a method of suppressing an immune response in a subject comprising administering a polypeptide complex of the disclosure to the subject such that an immune response in the subject is reduced while no undesired side effects are presented. For example, the subject is a mammal. In a specific embodiment, the subject is a human.

The term “suppressing an immune response” or its grammatical variations, means reducing any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of suppression of immune response include increased Treg cell activity and proliferation. The suppression of immune response can be assessed using a number of in vitro or in vivo measurements known to those skilled in the art, including, but not limited to, Treg lymphocyte assays, release of cytokines (for example IL-15 production or IFN-γ production), regression of autoimmune disease, survival of autoimmune animals, self-antibody production, immune cell proliferation, and cytotoxicity. Typically, methods of the disclosure enhance the immune response by a mammal when compared to the immune response by an untreated mammal or a mammal not treated using the methods as disclosed herein. In one embodiment, the immune response is cytokine production, particularly IFN-γ production or IL-17 production. In another embodiment, the immune response is suppressing B cell activity.

The polypeptide complexes as disclosed herein may be used alone as a monotherapy, or more often, used in combination with cell immunotherapies, gene therapy, targeted therapies or chemical therapies.

### Pharmaceutical Packs and Kits

Pharmaceutical packs and kits comprising one or more containers, comprising one or more doses of the polypeptide complexes are also provided. In certain embodiments, a unit dosage is provided wherein the unit dosage contains a predetermined amount of a composition comprising, for example, the polypeptide complexes, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present disclosure also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the polypeptide complexes of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed polypeptide complexes, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the polypeptide complexes and any optional components to a patient, e.g., one or more needles, I. V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

The following Table A provides a description of the polypeptide complex constructs and Tables B-C provide the sequences of IL-2 variants and each chain of the polypeptide complex herein. The designation “W3XX115-T2U0.E44-[N].UIGG4V322” (may be abbreviated as “T2U0.E44-[N]”) indicates the structure of the polypeptide complexes: [n] is the numbering, “E44” INDICATES THE format as shown in FIG. 1 COMPRISING TWO HEAVY CHAINS AND ONE LIGHT CHAIN. The designation “W3XX115-T2.Z20-[n].uIgG4V322” (may be abbreviated as “T2.Z20-[n]” or “Z20-[n]”) indicates that the polypeptide complex is in “Z20” format as shown in FIG. 1, comprising TWO CHAINS EACH COMPRISING A IL-2. W3XX115-T2U10.Z73-[N].UIGG4V322 indicates that the polypeptide complex is in “Z73” format as shown in FIG. 1, comprising ONE CHAIN COMPRISING A IL-2 VARIANT AND THE OTHER CHAIN COMPRISING A VHH. “uIgG4V322” REFERS TO IGG4 FC WITH F234A/L235A MUTATION (EU NUMBERING). “uIgG1V320” REFERS TO IGG1 FC WITH L234A/L235A MUTATION. THE PREFIX “W3xx115-” CAN BE OMITTED THROUGHOUT THE SPECIFICATION FOR SIMPLICITY.

SEQ ID NOS: 1-14, 79-103 AND 111-114 REFER TO THE AMINO ACID SEQUENCES OF IL-2 DOMAIN; SEQ ID NOS: 15-34, 38-78 AND 104-110 REFER TO THE AMINO ACID SEQUENCES OF THE POLYPEPTIDE COMPLEXES, INCLUDING THE BENCHMARK ANTIBODIES USED IN THE EXAMPLES; SEQ ID Nos: 35-37 REFER TO THE ECD SEQUENCE OF IL-2RA, B AND T. “FUSED ON N TERMINAL WITH NO LINKER” INDICATES THAT THE IL-2 DOMAIN IS DIRECTLY LINKED TO THE HINGE REGION.

TABLE A
Structure of the IL-2 comprising polypeptide complexes

| | | | 2ND | |
|---|---|---|---|---|
| | | 1ST CHAIN | CHAIN | |
| | | (OR 1ST | (OR 2ND | |
| | | HEAVY | HEAVY | 3RD CHAIN (OR |
| | FORMAT | CHAIN) | CHAIN) | LIGHT CHAIN) |
| W3XX115- | E44 | IL-2 VARIANT + | HEAVY | LIGHT CHAIN |
| T2U0.E44- | | Fc | CHAIN OF | OF ANTIGEN |
| [N].UIGG4V322 | | SEQ ID Nos: | ANTIGEN | BINDING FAB |
| N = 6, 26, 40-49 | | 17-28 | BINDING | SEQ ID NO: |
| | | | FAB + FC | 34 |
| | | | SEQ ID | |
| | | | NO: 33 | |
| W3XX115- | E44 | IL-2 VARIANT + | HEAVY | LIGHT CHAIN |
| T2U3.E44- | | Fc | CHAIN OF | OF ANTIGEN |
| [N].UIGG4V322 | | SEQ ID Nos: | ANTIGEN | BINDING FAB |
| N = 6, 26, 15, 20, | | 52-56 | BINDING | SEQ ID NO: 58 |
| 33 | | | FAB + FC | |
| | | | SEQ ID | |
| | | | NO: 57 | |
| W3XX115- | Z20 | IL-2 VARIANT + | IL-2 | NONE |
| T2.Z20- | | Fc | VARIANT + | |
| [N].UIGG4V322 | | SEQ ID Nos: | Fc | |
| N = 1, 2, 4, 5 | | 29-32 | SEQ ID | |
| | | | Nos: 29-32 | |
| W3XX115- | Z73 | IL-2 VARIANT + | ANTIGEN | NONE |
| T2U10.Z73- | | Fc | BINDING | |
| [N].UIGG4V322 | | SEQ ID Nos: | VHH + Fc | |
| N = 50-75 | | 59-74, 76-78, | SEQ ID | |
| | | 104-110 | No: 75 | |

TABLE B
AMINO ACID SEQUENCES OF WILD-TYPE IL-2 AND IL-2 VARIANT SEQUENCES

| SEQ ID | | |
|---|---|---|
| NO: | Sequence | Description |
| 1 | APTSSSTKKTQLQLEHLLLDLQMILNGINNYKNPKLTRML | WT IL-2 |
| | TFKFYMPKKATELKHLQCLEEELKPLEEVLNLAQSKNFHL | |
| | RPRDLISNINVIVLELKGSETTFMCEYADETATIVEFLNRWI | |
| | TFCQSIISTLT | |
| 2 | APTSSSTKKTQLQLEHLLLDLQMILNGINNYKNPKLTRML | C terminal, 4 amino acid |
| | TFKFYMPKKATELKHLQCLEEELKPLEEVLNLAQSKNFHL | truncation |
| | RPRDLISNINVIVLELKGSETTFMCEYADETATIVEFLNRWI | |
| | TFCQSII | |
| 3 | APTSSSTKKTQLQLEHLLLDLQMILNGINNYKNPKLTRML | C terminal, 4 amino acid |
| | TFKFYMPKKATELKHLQCLEEELKPLEEVLNLAQSKNFHL | truncation and I129L |
| | RPRDLISNINVIVLELKGSETTFMCEYADETATIVEFLNRWI | substitution |
| | TFCQSIL | |
| 4 | APTSSSTKKTQLQLEHLLLDLQMILNGINNYKNPKLTRML | C terminal truncation of 4 |
| | TFKFYMPKKATELKHLQCLEEELKPLEEVLNLAQSKNFHL | amino acids and E110R |
| | RPRDLISNINVIVLELKGSETTFMCEYADRTATIVEFLNRWI | substitution |
| | TFCQSII | |
| 5 | APTSSSTKKTQLQLEHLLLDLQMILNGINNYKNPKLTRML | C terminal truncation of 4 |
| | TFKFYMPKKATELKHLQCLEEELKPLEEVLNLAQSKNFHL | amino acids and I122Y |
| | RPRDLISNINVIVLELKGSETTFMCEYADETATIVEFLNRW | substitution |
| | YTFCQSII | |
| 6 | APTSSSTKKTQLQLEHLLLDLQMILNGPNNYKNPKLTRML | C terminal truncation of 4 |
| | TFKFYMPKKATELKHLQCLEEELKPLEEVLNLAQSKNFHL | amino acids and I28P |
| | RPRDLISNINVIVLELKGSETTFMCEYADETATIVEFLNRWI | substitution |
| | TFCQSII | |
| 7 | APTSSSTKKTQLQLEHLLLDLQMILNGINNYDNPKLTRML | C terminal truncation of 4 |
| | TFKFYMPKKATELKHLQCLEEELKPLEEVLNLAQSKNFHL | amino acids and K32D |
| | RPRDLISNINVIVLELKGSETTFMCEYADETATIVEFLNRWI | substitution |
| | TFCQSII | |
| 8 | APTSSSTKKTQLQLEHLLLDLQMILNGINNYKNPKLTRML | C terminal truncation of 4 |
| | TFKFYMPKKATGLKHLQCLEEELKPLEEVLNLAQSKNFHL | amino acids and E52G |
| | RPRDLISNINVIVLELKGSETTFMCEYADETATIVEFLNRWI | substitution |
| | TFCQSII | |
| 9 | APTSSSTKKTQLQLEHLLLDLQMILNGINNYKNPKLTRML | C terminal truncation of 4 |
| | TFKFYMPKKATELKHLQCLEEELKPLEEVLNLAQSRNFHL | amino acids and K76R |
| | RPRDLISNINVIVLELKGSETTFMCEYADETATIVEFLNRWI | substitution |
| | TFCQSII | |
| 10 | APTSSSTKKTQLQLEHLLLDLQMILNGINNYKNPKLTRML | C terminal truncation of 4 |
| | TFKFYMPKKATELKHLQCLEEELKPLEEVLNLAQSKNGH | amino acids and F78G |
| | LRPRDLISNINVIVLELKGSETTFMCEYADETATIVEFLNR | substitution |
| | WITFCQSII | |
| 11 | APTSSSTKKTQLQLEHLLLDLQMILNGINNYKNPKLTRML | C terminal truncation of 4 |
| | TFKFYMPKKATELKHLQCLEEELKPLEEVLNLAQSKNFHL | amino acids and P82Y |
| | RYRDLISNINVIVLELKGSETTFMCEYADETATIVEFLNRW | substitution |
| | ITFCQSII | |
| 12 | APTSSSTKKTQLQLEHLLLDLQMILNGINNYKNPKLTRML | C terminal truncation of 4 |
| | TFKFYMPKKATELKHLQCLEEELKPLEEVLNLAQSKNFHL | amino acids, E110R and |
| | RPRDLISNINVIVLELKGSETTFMCEYADRTATIVEFLNRWI | I129L substitution |
| | TFCQSIL | |
| 13 | APTSSSTKKTQLQLEHLLLDLQMILNGINNYDNPKLTRML | C terminal truncation of 4 |
| | TFKFYMPKKATELKHLQCLEEELKPLEEVLNLAQSKNFHL | amino acids, K32D and |
| | RPRDLISNINVIVLELKGSETTFMCEYADETATIVEFLNRWI | I129L substitutions |
| | TFCQSIL | |
| 14 | APTSSSTKKTQLQLEHLLLDLQMILNGINNYKNPKLTRML | F42V/Y45A/L72G |
| | TVKFAMPKKATELKHLQCLEEELKPLEEVLNGAQSKNFH | |
| | LRPRDLISNINVIVLELKGSETTFMCEYADETATIVEFLNR | |
| | WITFCQSIISTLT | |
| 79 | APTSSSTKKTQLQLEHLLLDLQMILNGINNYKNPKLTRML | C terminal truncation of 4 |
| | TVKFYMPKKATELKHLQCLEEELKPLEEVLNLAQSKNFH | amino acids and F42I |
| | LRPRDLISNINVIVLELKGSETTFMCEYADETATIVEFLNR | substitution |
| | WITFCQSII | |
| 80 | APTSSSTKKTQLQLEHLLLDLQMILNGINNYKNPKLTRML | C terminal truncation of 4 |
| | TIKFYMPKKATELKHLQCLEEELKPLEEVLNLAQSKNFHL | amino acids, F42I and |
| | RPRDLISNINVIVLELKGSETTFMCEYADETATIVEFLNRWI | I129L substitutions |
| | TFCQSIL | |
| 81 | APTSSSTKKTQLQLEHLLLDLQMILNGINNYKNPKLTRML | C terminal truncation of 4 |
| | TIKFYMPKKATELKHLQCLEEELKPLEEVLNLAQSKNFHL | amino acids, E110R, F42I |
| | RPRDLISNINVIVLELKGSETTFMCEYADRTATIVEFLNRWI | and I129L substitutions |
| | TFCQSIL | |
| 82 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, K32D, F42I |
| | RPRDLISNINVIVLELKGSETTFMCEYADETATIVEFLNRWI | and I129L substitutions |
| | TFCQSIL | |
| 83 | APTSSSTKKTQLQLEHLRLDLEMILNGINNYKNPKLTRML | L18R, Q22E, F42I and |
| | TIKFYMPKKATELKHLQCLEEELKPLEEVLNLAQSKNFHL | Q126K substitutions |
| | RPRDLISNINVIVLELKGSETTFMCEYADETATIVEFLNRWI | |
| | TFCKSIISTLT | |
| 84 | APTSSSTKKTQLQLEHLLHDLQMILNGINNYKNPKLTRML | L19H, F42I, C125I and |
| | TIKFYMPKKATELKHLQCLEEELKPLEEVLNLAQSKNFHL | Q126E substitutions |
| | RPRDLISNINVIVLELKGSETTFMCEYADETATIVEFLNRWI | |
| | TFIESIISTLT | |
| 85 | APASSSTKKTQLQLEHLLLNLQMILNGINNYKNPKLTRML | T3A, D20N, F42I, N71K |
| | TIKFYMPKKATELKHLQCLEEELKPLEEVLKLAQSKNFHL | and C125S substitutions |
| | RPRDLISNINVIVLELKGSETTFMCEYADETATIVEFLNRWI | |
| | TFSQSIISTLT | |
| 86 | APTSSSTKKTQLDLEHLLLDLQMILNGINNYDNPKLTRML | C terminal truncation of 4 |
| | TIKFYMPKKATELKHLQCLEEELKPLEEVLNLAQSKNFHL | amino acids, Q13D, K32D, |
| | RPRDLISNINVIVLELKGSETTFMCEYADETATIVEFLNRWI | F42I and I129L |
| | TFCQSIL | substitutions |
| 87 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, D84K, K32D, |
| | RPRKLISNINVIVLELKGSETTFMCEYADETATIVEFLNRWI | F42I and I129L |
| | TFCQSIL | substitutions |
| 88 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, S87R, K32D, |
| | RPRDLIRNINVIVLELKGSETTFMCEYADETATIVEFLNRW | F42I and I129L |
| | ITFCQSIL | substitutions |
| 89 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, N88K, K32D, |
| | RPRDLISKINVIVLELKGSETTFMCEYADETATIVEFLNRWI | F42I and I129L |
| | TFCQSIL | substitutions |
| 90 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, V91E, K32D, |
| | RPRDLISNINEIVLELKGSETTFMCEYADETATIVEFLNRWI | F42I and I129L |
| | TFCQSIL | substitutions |
| 91 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, 192R, K32D, |
| | RPRDLISNINVRVLELKGSETTFMCEYADETATIVEFLNRW | F42I and I129L |
| | ITFCQSIL | substitutions |
| 92 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, 192D, K32D, |
| | RPRDLISNINVDVLELKGSETTFMCEYADETATIVEFLNRW | F42I and I129L |
| | ITFCQSIL | substitutions |
| 93 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, L94D, K32D, |
| | RPRDLISNINVIVDELKGSETTFMCEYADETATIVEFLNRW | F42I and I129L |
| | ITFCQSIL | substitutions |
| 94 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, E95R, K32D, |
| | RPRDLISNINVIVLRLKGSETTFMCEYADETATIVEFLNRWI | F42I and I129L |
| | TFCQSIL | substitutions |
| 95 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, N119R, |
| | RPRDLISNINVIVLELKGSETTFMCEYADETATIVEFLRRWI | K32D, F42I and I129L |
| | TFCQSIL | substitutions |
| 96 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, T123K, |
| | RPRDLISNINVIVLELKGSETTFMCEYADETATIVEFLNRWI | K32D, F421 and I129L |
| | KFCQSIL | substitutions |
| 97 | APTSSSTKKTQLRLEHLLLDLQMILNGINNYDNPKLTRML | C terminal truncation of 4 |
| | TIKFYMPKKATELKHLQCLEEELKPLEEVLNLAQSKNFHL | amino acids, Q13R, K32D, |
| | RPRDLISNINVIVLELKGSETTFMCEYADETATIVEFLNRWI | F42I and I129L |
| | TFCQSIL | substitutions |
| 98 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, D84T, K32D, |
| | RPRTLISNINVIVLELKGSETTFMCEYADETATIVEFLNRWI | F42I and I129L |
| | TFCQSIL | substitutions |
| 99 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, S87I, K32D, |
| | RPRDLIININVIVLELKGSETTFMCEYADETATIVEFLNRWI | F42I and I129L |
| | TFCQSIL | substitutions |
| 100 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, V91S, K32D, |
| | RPRDLISNINSIVLELKGSETTFMCEYADETATIVEFLNRWI | F42I and I129L |
| | TFCQSIL | substitutions |
| 101 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, E95Y, K32D, |
| | RPRDLISNINVIVLYLKGSETTFMCEYADETATIVEFLNRW | F42I and I129L |
| | ITFCQSIL | substitutions |
| 102 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, N119Q, |
| | RPRDLISNINVIVLELKGSETTFMCEYADETATIVEFLQRWI | K32D, F421 and I129L |
| | TFCQSIL | substitutions |
| 103 | APTSSSTKKTQLQLEHLLLDLQMILNGINNYDNPKLTRML | C terminal truncation of 4 |
| | TIKFYMPKKATELKHLQCLEEELKPLEEVLNLAQSKNFHL | amino acids, T123V, |
| | RPRDLISNINVIVLELKGSETTFMCEYADETATIVEFLNRWI | K32D, F421 and I129L |
| | VFCQSIL | substitutions |
| 111 | APTSSSTKKTQLQLEHLLLDLQMILNGINNYKNPKLTWML | C terminal truncation of 4 |
| | TFKFYMPKKATELKHLQCLEEELKPLEEVLNLAQSKNFHL | amino acids, R38W |
| | RPRDLISNINVIVLELKGSETTFMCEYADETATIVEFLNRWI | substitution |
| | TFCQSIL | |
| 112 | APTSSSTKKTQLQLEHLRLDLEMILNGINNYKNPKLTRML | L18R/Q22E/Q126K |
| | TFKFYMPKKATELKHLQCLEEELKPLEEVLNLAQSKNFHL | |
| | RPRDLISNINVIVLELKGSETTFMCEYADETATIVEFLNRWI | |
| | TFCKSIISTLT | |
| 113 | APTSSSTKKTQLQLEHLLHDLQMILNGINNYKNPKLTRML | L19H/C125I/Q126E |
| | TFKFYMPKKATELKHLQCLEEELKPLEEVLNLAQSKNFHL | |
| | RPRDLISNINVIVLELKGSETTFMCEYADETATIVEFLNRWI | |
| | TFIESIISTLT | |
| 114 | APASSSTKKTQLQLEHLLLNLQMILNGINNYKNPKLTRML | T3A/D20N/N71K/C125S |
| | TFKFYMPKKATELKHLQCLEEELKPLEEVLKLAQSKNFHL | |
| | RPRDLISNINVIVLELKGSETTFMCEYADETATIVEFLNRWI | |
| | TFSQSIISTLT | |

TABLE C
Amino acid sequences of each chain of the polypeptide complexes

| | | |
|---|---|---|
| 15 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNFHL | 1.uIgG4V322, 1st heavy |
| | RPRDLISNINVIVLELKGSETTFMCEYADETATIVEFLNRWI | chain |
| | TFCQSIISTLTESKYGPPCPPCPAPEAAGGPSVFLFPPKPKD | WT IL2 on N-terminal |
| | TLMISRTPEVTCVVVDVSQEDPEVQFNWYVDGVEVHNAK | with no linker |
| | TKPREEQFNSTYRVVSVLTVLHQDWLNGKEYKCKVSNK | |
| | GLPSSIEKTISKAKGQPREPQVYTLPPCQEEMTKNQVSLW | |
| | CLVKGFYPSDIAVEWESNGQPENNYKTTPPVLDSDGSFFL | |
| | YSRLTVDKSRWQEGNVFSCSVMHEALHNHYTQKSLSLSL | |
| | G | |
| 16 | APTSSSTKKTQLQLEHLLLDLQMILNGINNYKNPKLTRML | W3XX115-T3U0.E44- |
| | TVKFAMPKKATELKHLQCLEEELKPLEEVLNGAQSKNFH | 1.uIgG4V322, 1st heavy |
| | LRPRDLISNINVIVLELKGSETTFMCEYADETATIVEFLNR | chain |
| | WITFCQSIISTLT | IL2 variant on N-terminal |
| | ESKYGPPCPPCPAPEAAGGPSVFLFPPKPKDTLMISRTPEV | with no linker |
| | TCVVVDVSQEDPEVQFNWYVDGVEVHNAKTKPREEQFN | |
| | STYRVVSVLTVLHQDWLNGKEYKCKVSNKGLPSSIEKTIS | |
| | KAKGQPREPQVYTLPPCQEEMTKNQVSLWCLVKGFYPSD | |
| | IAVEWESNGQPENNYKTTPPVLDSDGSFFLYSRLTVDKSR | |
| | WQEGNVFSCSVMHEALHNHYTQKSLSLSLG | |
| 17 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNFHL | 6.uIgG4V322, 1st heavy |
| | RPRDLISNINVIVLELKGSETTFMCEYADETATIVEFLNRWI | chain |
| | TFCQSIIESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | fused on N-terminal with |
| | SIEKTISKAKGQPREPQVYTLPPCQEEMTKNQVSLWCLVK | no linker |
| | GFYPSDIAVEWESNGQPENNYKTTPPVLDSDGSFFLYSRL | |
| | TVDKSRWQEGNVFSCSVMHEALHNHYTQKSLSLSLG | |
| 18 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNFHL | 26.uIgG4V322, 1st heavy |
| | RPRDLISNINVIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion |
| | REEQFNSTYRVVSVLTVLHQDWLNGKEYKCKVSNKGLPS | and I129L, fused on N- |
| | SIEKTISKAKGQPREPQVYTLPPCQEEMTKNQVSLWCLVK | terminal with no linker |
| | GFYPSDIAVEWESNGQPENNYKTTPPVLDSDGSFFLYSRL | |
| | TVDKSRWQEGNVFSCSVMHEALHNHYTQKSLSLSLG | |
| 19 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNFHL | 40.uIgG4V322, 1st heavy |
| | RPRDLISNINVIVLELKGSETTFMCEYADRTATIVEFLNRWI | chain |
| | TFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | E110R, fused on N- |
| | STYRVVSVLTVLHQDWLNGKEYKCKVSNKGLPSSIEKTIS | terminal with no linker |
| | KAKGQPREPQVYTLPPCQEEMTKNQVSLWCLVKGFYPSD | |
| | IAVEWESNGQPENNYKTTPPVLDSDGSFFLYSRLTVDKSR | |
| | WQEGNVFSCSVMHEALHNHYTQKSLSLSLG | |
| 20 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNFHL | 41.uIgG4V322, 1st heavy |
| | RPRDLISNINVIVLELKGSETTFMCEYADRTATIVEFLNRWI | chain |
| | TFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | I122Y, fused on N- |
| | STYRVVSVLTVLHQDWLNGKEYKCKVSNKGLPSSIEKTIS | terminal with no linker |
| | KAKGQPREPQVYTLPPCQEEMTKNQVSLWCLVKGFYPSD | |
| | IAVEWESNGQPENNYKTTPPVLDSDGSFFLYSRLTVDKSR | |
| | WQEGNVFSCSVMHEALHNHYTQKSLSLSLG | |
| 21 | APTSSSTKKTQLQLEHLLLDLQMILNGPNNYKNPKLTRML | W3XX115-T2U0.E44- |
| | TFKFYMPKKATELKHLQCLEEELKPLEEVLNLAQSKNFHL | 42.uIgG4V322, 1st heavy |
| | RPRDLISNINVIVLELKGSETTFMCEYADETATIVEFLNRWI | chain |
| | TFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | I28P, fused on N-terminal |
| | STYRVVSVLTVLHQDWLNGKEYKCKVSNKGLPSSIEKTIS | with no linker |
| | KAKGQPREPQVYTLPPCQEEMTKNQVSLWCLVKGFYPSD | |
| | IAVEWESNGQPENNYKTTPPVLDSDGSFFLYSRLTVDKSR | |
| | WQEGNVFSCSVMHEALHNHYTQKSLSLSLG | |
| 22 | APTSSSTKKTQLQLEHLLLDLQMILNGINNYDNPKLTRML | W3XX115-T2U0.E44- |
| | TFKFYMPKKATELKHLQCLEEELKPLEEVLNLAQSKNFHL | 43.uIgG4V322, 1st heavy |
| | RPRDLISNINVIVLELKGSETTFMCEYADETATIVEFLNRWI | chain |
| | TFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | K32D, fused on N-terminal |
| | STYRVVSVLTVLHQDWLNGKEYKCKVSNKGLPSSIEKTIS | with no linker |
| | KAKGQPREPQVYTLPPCQEEMTKNQVSLWCLVKGFYPSD | |
| | LAVEWESNGQPENNYKTTPPVLDSDGSFFLYSRLTVDKSR | |
| | WQEGNVFSCSVMHEALHNHYTQKSLSLSLG | |
| 23 | APTSSSTKKTQLQLEHLLLDLQMILNGINNYKNPKLTRML | W3XX115-T2U0.E44- |
| | TFKFYMPKKATGLKHLQCLEEELKPLEEVLNLAQSKNFHL | 44.uIgG4V322, 1st heavy |
| | RPRDLISNINVIVLELKGSETTFMCEYADETATIVEFLNRWI | chain |
| | TFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | E52G, fused on N-terminal |
| | STYRVVSVLTVLHQDWLNGKEYKCKVSNKGLPSSIEKTIS | with no linker |
| | KAKGQPREPQVYTLPPCQEEMTKNQVSLWCLVKGFYPSD | |
| | IAVEWESNGQPENNYKTTPPVLDSDGSFFLYSRLTVDKSR | |
| | WQEGNVFSCSVMHEALHNHYTQKSLSLSLG | |
| 24 | APTSSSTKKTQLQLEHLLLDLQMILNGINNYKNPKLTRML | W3XX115-T2U0.E44- |
| | TFKFYMPKKATELKHLQCLEEELKPLEEVLNLAQSRNFHL | 45.uIgG4V322, 1st heavy |
| | RPRDLISNINVIVLELKGSETTFMCEYADETATIVEFLNRWI | chain |
| | TFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | K76R, fused on N-terminal |
| | STYRVVSVLTVLHQDWLNGKEYKCKVSNKGLPSSIEKTIS | with no linker |
| | KAKGQPREPQVYTLPPCQEEMTKNQVSLWCLVKGFYPSD | |
| | IAVEWESNGQPENNYKTTPPVLDSDGSFFLYSRLTVDKSR | |
| | WQEGNVFSCSVMHEALHNHYTQKSLSLSLG | |
| 25 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNGH | 46.uIgG4V322, 1st heavy |
| | LRPRDLISNINVIVLELKGSETTFMCEYADETATIVEFLNR | chain |
| | WITFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | F78G, fused on N-terminal |
| | STYRVVSVLTVLHQDWLNGKEYKCKVSNKGLPSSIEKTIS | with no linker |
| | KAKGQPREPQVYTLPPCQEEMTKNQVSLWCLVKGFYPSD | |
| | IAVEWESNGQPENNYKTTPPVLDSDGSFFLYSRLTVDKSR | |
| | WQEGNVFSCSVMHEALHNHYTQKSLSLSLG | |
| 26 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNFHL | 47.uIgG4V322, 1st heavy |
| | RYRDLISNINVIVLELKGSETTFMCEYADETATIVEFLNRW | chain |
| | ITFCQSII | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | P82Y, fused on N-terminal |
| | STYRVVSVLTVLHQDWLNGKEYKCKVSNKGLPSSIEKTIS | with no linker |
| | KAKGQPREPQVYTLPPCQEEMTKNQVSLWCLVKGFYPSD | |
| | LAVEWESNGQPENNYKTTPPVLDSDGSFFLYSRLTVDKSR | |
| | WQEGNVFSCSVMHEALHNHYTQKSLSLSLG | |
| 27 | APTSSSTKKTQLQLEHLLLDLQMILNGINNYKNPKLTRML | W3XX115-T2U0.E44- |
| | TFKFYMPKKATELKHLQCLEEELKPLEEVLNLAQSKNFHL | 48.uIgG4V322, 1st heavy |
| | RPRDLISNINVIVLELKGSETTFMCEYADETATIVEFLNRW | chain |
| | YTFCQSIL | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | E110R and I129L, fused on |
| | STYRVVSVLTVLHQDWLNGKEYKCKVSNKGLPSSIEKTIS | N-terminal with no linker |
| | KAKGQPREPQVYTLPPCQEEMTKNQVSLWCLVKGFYPSD | |
| | LAVEWESNGQPENNYKTTPPVLDSDGSFFLYSRLTVDKSR | |
| | WQEGNVFSCSVMHEALHNHYTQKSLSLSLG | |
| 28 | APTSSSTKKTQLQLEHLLLDLQMILNGINNYDNPKLTRML | W3XX115-T2U0.E44- |
| | TFKFYMPKKATELKHLQCLEEELKPLEEVLNLAQSKNFHL | 49.uIgG4V322, 1st heavy |
| | RPRDLISNINVIVLELKGSETTFMCEYADETATIVEFLNRWI | chain |
| | TFCQSIL | WT IL2 with C-terminal |
| | ESKYGPPCPPCPAPEAAGGPSVFLFPPKPKDTLMISRTPEV | four amino acids deletion, |
| | TCVVVDVSQEDPEVQFNWYVDGVEVHNAKTKPREEQFN | K32D and I129L, fused on |
| | STYRVVSVLTVLHQDWLNGKEYKCKVSNKGLPSSIEKTIS | N-terminal with no linker |
| | KAKGQPREPQVYTLPPCQEEMTKNQVSLWCLVKGFYPSD | |
| | LAVEWESNGQPENNYKTTPPVLDSDGSFFLYSRLTVDKSR | |
| | WQEGNVFSCSVMHEALHNHYTQKSLSLSLG | |
| 29 | APTSSSTKKTQLQLEHLLLDLQMILNGINNYKNPKLTRML | W3XX115-T2.Z20- |
| | TFKFYMPKKATELKHLQCLEEELKPLEEVLNLAQSKNFHL | 1.uIgG4V322, one chain |
| | RPRDLISNINVIVLELKGSETTFMCEYADETATIVEFLNRWI | WT IL2 with C-terminal |
| | TFCQSIIESKYGPPCPPCPAPEAAGGPSVFLFPPKPKDTLMI | four amino acids deletion, |
| | SRTPEVTCVVVDVSQEDPEVQFNWYVDGVEVHNAKTKP | fused on N-terminal with |
| | REEQFNSTYRVVSVLTVLHQDWLNGKEYKCKVSNKGLPS | no linker |
| | SIEKTISKAKGQPREPQVYTLPPSQEEMTKNQVSLTCLVKG | |
| | FYPSDIAVEWESNGQPENNYKTTPPVLDSDGSFFLYSRLT | |
| | VDKSRWQEGNVFSCSVMHEALHNHYTQKSLSLSLGK | |
| 30 | APTSSSTKKTQLQLEHLLLDLQMILNGINNYKNPKLTRML | W3XX115-T2.Z20- |
| | TFKFYMPKKATELKHLQCLEEELKPLEEVLNLAQSKNFHL | 2. uIgG4V322, one chain |
| | RPRDLISNINVIVLELKGSETTFMCEYADETATIVEFLNRWI | WT IL2 with C-terminal |
| | TFCQSILESKYGPPCPPCPAPEAAGGPSVFLFPPKPKDTLMI | four amino acids deletion |
| | SRTPEVTCVVVDVSQEDPEVQFNWYVDGVEVHNAKTKP | and I129L, fused on N- |
| | REEQFNSTYRVVSVLTVLHQDWLNGKEYKCKVSNKGLPS | terminal with no linker |
| | SIEKTISKAKGQPREPQVYTLPPSQEEMTKNQVSLTCLVKG | |
| | FYPSDIAVEWESNGQPENNYKTTPPVLDSDGSFFLYSRLT | |
| | VDKSRWQEGNVFSCSVMHEALHNHYTQKSLSLSLGK | |
| 31 | APTSSSTKKTQLQLEHLLLDLQMILNGINNYKNPKLTRML | W3XX115-T2.Z20- |
| | TFKFYMPKKATELKHLQCLEEELKPLEEVLNLAQSKNFHL | 4.uIgG4V322, one chain |
| | RPRDLISNINVIVLELKGSETTFMCEYADRTATIVEFLNRW | WT IL2 with C-terminal |
| | YTFCQSIL | four amino acids deletion, |
| | ESKYGPPCPPCPAPEAAGGPSVFLFPPKPKDTLMISRTPEV | E110R and I129L, fused on |
| | TCVVVDVSQEDPEVQFNWYVDGVEVHNAKTKPREEQFN | N-terminal with no linker |
| | STYRVVSVLTVLHQDWLNGKEYKCKVSNKGLPSSIEKTIS | |
| | KAKGQPREPQVYTLPPSQEEMTKNQVSLTCLVKGFYPSDI | |
| | AVEWESNGQPENNYKTTPPVLDSDGSFFLYSRLTVDKSR | |
| | WQEGNVFSCSVMHEALHNHYTQKSLSLSLGK | |
| 32 | APTSSSTKKTQLQLEHLLLDLQMILNGINNYDNPKLTRML | W3XX115-T2.Z20- |
| | TFKFYMPKKATELKHLQCLEEELKPLEEVLNLAQSKNFHL | 5.uIgG4V322, one chain |
| | RPRDLISNINVIVLELKGSETTFMCEYADETATIVEFLNRWI | WT IL2 with C-terminal |
| | TFCQSIL | four amino acids deletion, |
| | ESKYGPPCPPCPAPEAAGGPSVFLFPPKPKDTLMISRTPEV | K32D and I129L, fused on |
| | TCVVVDVSQEDPEVQFNWYVDGVEVHNAKTKPREEQFN | N-terminal with no linker |
| | STYRVVSVLTVLHQDWLNGKEYKCKVSNKGLPSSIEKTIS | |
| | KAKGQPREPQVYTLPPSQEEMTKNQVSLTCLVKGFYPSDI | |
| | AVEWESNGQPENNYKTTPPVLDSDGSFFLYSRLTVDKSR | |
| | WQEGNVFSCSVMHEALHNHYTQKSLSLSLGK | |
| 33 | EIQLQQSGPELVKPGASVKVSCKASGYSFTDYNMYWVKQ | E44 uIgG4V322 format, |
| | SHGESLEWIGYIDPYNGGTRYNQKFKGKATLTVDKSSSTA | 2^{nd} heavy chain, |
| | FMHLNSLTSEDSAVYYCARNGHWDGAWFAYWGQGTLV | comprising heavy chain of |
| | TVSAASTKGPSVFPLAPCSRSTSESTAALGCLVKDYFPEPV | a Fab |
| | TVSWNSGALTSGVHTFPAVLQSSGLYSLSSVVTVPSSSLG | |
| | TKTYTCNVDHKPSNTKVDKRVESKYGPPCPPCPAPEAAG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVCTLPPSQ | |
| | EEMTKNQVSLSCAVKGFYPSDIAVEWESNGQPENNYKTT | |
| | PPVLDSDGSFFLVSRLTVDKSRWQEGNVFSCSVMHEALH | |
| | NHYTQKSLSLSLG | |
| 34 | DIQMNQSPSSLSASLGDTITITCHASQNINVWLSWYQQKP | Light chain for E44 |
| | GNIPKLLIYKASNLHTGVPSRFSGSRSGTGFTLTISSLQPEDI | format, comprising light |
| | ATYYCHQGQSYPFTFGSGTKLEIKRTVAAPSVFIFPPSDEQ | chain of a Fab |
| | LKSGTASVVCLLNNFYPREAKVQWKVDNALQSGNSQESV | |
| | TEQDSKDSTYSLSSTLTLSKADYEKHKVYACEVTHQGLSS | |
| | PVTKSFNRGEC | |
| 38 | APTSSSTSSSTAEAQQQQQHLEQLRMDLEELLSRMENYRN | W3XX115-BMK7, E44 |
| | LKLPRMLTFKFYLPKQATELKDLOCLEDELGPLRHVLDLT | format, 1st heavy chain |
| | OSKSFQLEDAENFISNIRVTVVKLKGSDNTFECOFDDESAT | mouse IL2 with 41-46, 91, |
| | VVDFLRRWIAFCHSIISTSPOESKYGPPCPPCPAPEFLGGPS | 101, 141 & 169 amino acids |
| | VFLFPPKPKDTLMISRTPEVTCVVVDVSQEDPEVQFNWYV | deletion and L53R, |
| | DGVEVHNAKTKPREEQFNSTYRVVSVLTVLHQDWLNGK | Q162H, fused on N- |
| | EYKCKVSNKGLPSSIEKTISKAKGQPREPQVYTLPPCQEEM | terminal with no linker, |
| | TKNQVSLWCLVKGFYPSDIAVEWESNGQPENNYKTTPPV | FALA mutation on hlgG4 |
| | LDSDGSFFLYSRLTVDKSRWQEGNVFSCSVMHEALHNHY | Fc |
| | TQKSLSLSLG | |
| 39 | APTSSSTKKTQLQLEHLRLDLEMILNGINNYKNPKLTRML | W3XX115-BMK8, E44 |
| | TFKFYMPKKA | format, 1st heavy chain |
| | TELKHLQCLEEELKPLEEVLNLAQSKNFHLRPRDLISNINV | WT IL2 with L18R, Q22E, |
| | IVLELKGSETTFMCEYADETATIVEFLNRWITFCKSIISTLT | Q126K fused on N- |
| | ESKYGPPCPPCPAPEFLGGPSVFLFPPKPKDTLMISRTPEVT | terminal with no linker, |
| | CVVVDVSQEDPEVQFNWYVDGVEVHNAKTKPREEQFNS | FALA mutation on hlgG4 |
| | TYRVVSVLTVLHQDWLNGKEYKCKVSNKGLPSSIEKTISK | Fc |
| | AKGQPREPQVYTLPPCQEEMTKNQVSLWCLVKGFYPSDI | |
| | AVEWESNGQPENNYKTTPPVLDSDGSFFLYSRLTVDKSR | |
| | WQEGNVFSCSVMHEALHNHYTQKSLSLSLG | |
| 40 | QVQLVQSGAEVKKPGSSVKVSCKASGFTFTTYYISWVRQ | W3XX115-U3T3.F114- |
| | APGQGLEYLGYINMGSGGTNYNEKFKGRVTITADKSTST | 1.uIgG4V322, 1st heavy |
| | AYMELSSLRSEDTAVYYCAIIGYFDYWGQGTMVTVSSAS | chain |
| | TKGPSVFPLAPCSRSTSESTAALGCLVKDYFPEPVTVSWNS | IL2 variant on C-terminal |
| | GALTSGVHTFPAVLQSSGLYSLSSVVTVPSSSLGTKTYTCN | with no linker |
| | VDHKPSNTKVDKRVESKYGPPCPPCPAPEAAGGPSVFLFP | |
| | PKPKDTLMISRTPEVTCVVVDVSQEDPEVQFNWYVDGVE | |
| | VHNAKTKPREEQFNSTYRVVSVLTVLHQDWLNGKEYKC | |
| | KVSNKGLPSSIEKTISKAKGQPREPQVYTLPPCQEEMTKN | |
| | QVSLWCLVKGFYPSDIAVEWESNGQPENNYKTTPPVLDS | |
| | DGSFFLYSRLTVDKSRWQEGNVFSCSVMHEALHNHYTQK | |
| | SLSLSLGGGGGSGGGGSGGGGSGGGGSAPTSSSTKKTQLQ | |
| | LEHLLLDLQMILNGINNYKNPKLTRMLTAKFAMPKKATE | |
| | LKHLQCLEEELKPLEEVLNGAQSKNFHLRPRDLISNINVIV | |
| | LELKGSETTFMCEYADETATIVEFLNRWITFCQSIISTLT | |
| 41 | QVQLVQSGAEVKKPGSSVKVSCKASGFTFTTYYISWVRQ | W3XX115-U3T3.F114- |
| | APGQGLEYLGYINMGSGGTNYNEKFKGRVTITADKSTST | 1.uIgG4V322, 2nd heavy |
| | AYMELSSLRSEDTAVYYCAIIGYFDYWGQGTMVTVSSAS | chain, comprising heavy |
| | TKGPSVFPLAPCSRSTSESTAALGCLVKDYFPEPVTVSWNS | chain of a PD-1 binding |
| | GALTSGVHTFPAVLQSSGLYSLSSVVTVPSSSLGTKTYTCN | Fab (U3) |
| | VDHKPSNTKVDKRVESKYGPPCPPCPAPEAAGGPSVFLFP | |
| | PKPKDTLMISRTPEVTCVVVDVSQEDPEVQFNWYVDGVE | |
| | VHNAKTKPREEQFNSTYRVVSVLTVLHQDWLNGKEYKC | |
| | KVSNKGLPSSIEKTISKAKGQPREPQVCTLPPSQEEMTKNQ | |
| | VSLSCAVKGFYPSDIAVEWESNGQPENNYKTTPPVLDSDG | |
| | SFFLVSRLTVDKSRWQEGNVFSCSVMHEALHNHYTQKSL | |
| | SLSLGK | |
| 42 | DVVMTQSPLSLPVTLGQPASISCRSSQSLLDSDGGTYLYW | W3XX115-U3T3.F114- |
| | FQQRPGQSPRRLIYLVSTLGSGVPDRFSGSGSGTDFTLKISR | 1.uIgG4V322, light chain, |
| | VEAEDVGVYYCMQLTHWPYTFGQGTKLEIKRTVAAPSVF | comprising light chain of a |
| | IFPPSDEQLKSGTASVVCLLNNFYPREAKVQWKVDNALQS | PD-1 binding Fab (U3) |
| | GNSQESVTEQDSKDSTYSLSSTLTLSKADYEKHKVYACEV | |
| | THQGLSSPVTKSFNRGEC | |
| 43 | APTSSSTKKTQLQLEHLLLDLQMILNGINNYKNPKLTRML | W3XX115-BMK14 1st |
| | TFKFYMPKKATELKHLQCLEEELKPLEEVLNLAQSKNFHL | heavy chain |
| | RPRDLISDINVIVLELKGSETTFMCEYADETATIVEFLNRWI | IL2 variant on N-terminal |
| | TFCQSIISTLTESKYGPPCPPCPAPEAAGGPSVFLFPPKPKD | with no linker |
| | TLMISRTPEVTCVVVDVSQEDPEVQFNWYVDGVEVHNAK | |
| | TKPREEQFNSTYRVVSVLTVLHQDWLNGKEYKCKVSNK | |
| | GLPSSIEKTISKAKGQPREPQVYTLPPCQEEMTKNQVSLW | |
| | CLVKGFYPSDIAVEWESNGQPENNYKTTPPVLDSDGSFFL | |
| | YSRLTVDKSRWQEGNVFSCSVMHEALHNHYTQKSLSLSL | |
| | GK | |
| 44 | QVQLVQSGAEVKKPGSSVKVSCKASGGTFSSYAISWVRQ | W3XX115-BMK14, 2nd |
| | APGQGLEWMGLIIPMFDTAGYAQKFQGRVAITVDESTSTA | heavy chain |
| | YMELSSLRSEDTAVYYCARAEHSSTGTFDYWGQGTLVTV | |
| | SSASTKGPSVFPLAPCSRSTSESTAALGCLVKDYFPEPVTV | |
| | SWNSGALTSGVHTFPAVLQSSGLYSLSSVVTVPSSSLGTK | |
| | TYTCNVDHKPSNTKVDKRVESKYGPPCPPCPAPEAAGGPS | |
| | VFLFPPKPKDTLMISRTPEVTCVVVDVSQEDPEVQFNWYV | |
| | DGVEVHNAKTKPREEQFNSTYRVVSVLTVLHQDWLNGK | |
| | EYKCKVSNKGLPSSIEKTISKAKGQPREPQVCTLPPSQEEM | |
| | TKNQVSLSCAVKGFYPSDIAVEWESNGQPENNYKTTPPVL | |
| | DSDGSFFLVSRLTVDKSRWQEGNVFSCSVMHEALHNHYT | |
| | QKSLSLSLGK | |
| 45 | DIQMTQSPSSVSASVGDRVTITCRASQGISSWLAWYQQKP | Light chain for W3XX115- |
| | GKAPKLLISAASSLQSGVPSRFSGSGSGTDFTLTISSLQPED | BMK14 |
| | FATYYCQQANHLPFTFGGGTKVEIKRTVAAPSVFIFPPSDE | |
| | QLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |
| 46 | EVQLVESGGGLVKPGGSLELSCAASGFTFSSYWMSWVRQ | W327199-BMK1, 1st |
| | APEKGLEWVAAISPSGGSTYYADSVKGRFTISRDNAKNTL | heavy chain |
| | FLQMTSLRSEDTAMYYCAKESWGAYYDLWGQGTTVTVS | IL15 variant on C- |
| | SASTKGPSVFPLAPSSKSTSGGTAALGCLVKDYFPEPVTVS | terminal with linker |
| | WNSGALTSGVHTFPAVLQSSGLYSLSSVVTVPSSSLGTQT | |
| | YICNVNHKPSNTKVDKKVEPKSCDKTHTCPPCPAPEAAG | |
| | APSVFLFPPKPKDTLMISRTPEVTCVVVDVSHEDPEVKFN | |
| | WYVDGVEVHNAKTKPREEQYNSTYRVVSVLTVLHQDWL | |
| | NGKEYKCKVSNKALPAPIEKTISKAKGQPREPQVYTLPPC | |
| | REEMTKNQVSLSCAVKGFYPSDIAVEWESNGQPENNYKT | |
| | TPPVLDSDGSFFLVSKLTVDKSRWQQGNVFSCSVMHEAL | |
| | HNHYTQKSLSLSPGSGGGGSGGGGSGGGGAWVNVISDLK | |
| | KIEDLIQSMHIDATLYTESNVHPSCKVTAMKCFLLGLQRIS | |
| | LESGDASIHDTVENLIILANNSLSSNGNVTESGCKECEELE | |
| | EKNIKEFLQSFVHIVQMFINTS | |
| 47 | EVQLVESGGGLVKPGGSLELSCAASGFTFSSYWMSWVRQ | W327199-BMK1, 2nd |
| | APEKGLEWVAAISPSGGSTYYADSVKGRFTISRDNAKNTL | heavy chain |
| | FLQMTSLRSEDTAMYYCAKESWGAYYDLWGQGTTVTVS | |
| | SASTKGPSVFPLAPSSKSTSGGTAALGCLVKDYFPEPVTVS | |
| | WNSGALTSGVHTFPAVLQSSGLYSLSSVVTVPSSSLGTQT | |
| | YICNVNHKPSNTKVDKKVEPKSCDKTHTCPPCPAPEAAG | |
| | APSVFLFPPKPKDTLMISRTPEVTCVVVDVSHEDPEVKFN | |
| | WYVDGVEVHNAKTKPREEQYNSTYRVVSVLTVLHQDWL | |
| | NGKEYKCKVSNKALPAPIEKTISKAKGQPREPQVCTLPPSR | |
| | EEMTKNQVSLWCLVKGFYPSDIAVEWESNGQPENNYKTT | |
| | PPVLDSDGSFFLYSKLTVDKSRWQQGNVFSCSVMHEALH | |
| | NHYTQKSLSLSPGK | |
| 48 | DIVMTQSPSSLSVSAGDKVTMSCRASQGISSWLAWYQQK | Light chain for W327199- |
| | PWQPPKLLIYKASTLESGVPDRFTGSGSGTDFTLTISSVQA | BMK1 |
| | EDLAVYYCQQSYSTPWTFGGGTKLEIKGTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |
| 49 | QVQLVQSGAEVKKPGSSVKVSCKASGYTFTSYWINWVRQ | W327199-BMK2, 1st |
| | APGQGLEWMGNIYPGSSITNYAQKFQGRVTITADESTSTA | heavy chain |
| | YMELSSLRSEDTAVYYCARLTTGTFAYWGQGTLVTVSSA | |
| | STKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSCDKTHTCPPCPAPEAAGAP | |
| | SVFLFPPKPKDTLMISRTPEVTCVVVDVSHEDPEVKFNWY | |
| | VDGVEVHNAKTKPREEQYNSTYRVVSVLTVLHQDWLNG | |
| | KEYKCKVSNKALPAPIEKTISKAKGQPREPQVCTLPPSREE | |
| | MTKNQVSLWCLVKGFYPSDIAVEWESNGQPENNYKTTPP | |
| | VLDSDGSFFLYSKLTVDKSRWQQGNVFSCSVMHEALHNH | |
| | YTQKSLSLSPGK | |
| 50 | QVQLVQSGAEVKKPGSSVKVSCKASGYTFTSYWINWVRQ | W327199-BMK2, 2nd |
| | APGQGLEWMGNIYPGSSITNYAQKFQGRVTITADESTSTA | heavy chain |
| | YMELSSLRSEDTAVYYCARLTTGTFAYWGQGTLVTVSSA | IL15 variant on C- |
| | STKGPSVFPLAPSSKSTSGGTAALGCLVKDYFPEPVTVSW | terminal with no linker |
| | NSGALTSGVHTFPAVLQSSGLYSLSSVVTVPSSSLGTQTYI | |
| | CNVNHKPSNTKVDKKVEPKSCDKTHTCPPCPAPEAAGAP | |
| | SVFLFPPKPKDTLMISRTPEVTCVVVDVSHEDPEVKFNWY | |
| | VDGVEVHNAKTKPREEQYNSTYRVVSVLTVLHQDWLNG | |
| | KEYKCKVSNKALPAPIEKTISKAKGQPREPQVYTLPPCREE | |
| | MTKNQVSLSCAVKGFYPSDIAVEWESNGQPENNYKTTPP | |
| | VLDSDGSFFLVSKLTVDKSRWQQGNVFSCSVMHEALHNH | |
| | YTQKSLSLSPGSGGGGSGGGGSGGGGGWVNVISDLKKIE | |
| | DLIQSMHIDATLYTESNVHPSCKVTAMKCFLLGLQRISLES | |
| | GDASIHDTVQNLIILANNSLSSNGNVTESGCKECEELEEKN | |
| | IKEFLQSFVHIVQMFINTS | |
| 51 | DIQMTQSPSSLSASVGDRVTITCKSSQSLWDSGNQKNFLT | Light chain forW327199- |
| | WYQQKPGKAPKLLIYWTSYRESGVPSRFSGSGSGTDFTLT | BMK2 |
| | ISSLQPEDFATYYCONDYFYPLTFGGGTKVEIKRTVAAPSV | |
| | FIFPPSDEQLKSGTASVVCLLNNFYPREAKVQWKVDNALQ | |
| | SGNSQESVTEQDSKDSTYSLSSTLTLSKADYEKHKVYACE | |
| | VTHQGLSSPVTKSFNRGEC | |
| 52 | APTSSSTKKTQLQLEHLLLDLQMILNGINNYKNPKLTRML | W3XX115-T2U3.E44- |
| | TFKFYMPKKATELKHLQCLEEELKPLEEVLNLAQSKNFHL | 6.uIgG4V322, 1st heavy |
| | RPRDLISNINVIVLELKGSETTFMCEYADETATIVEFLNRWI | chain |
| | TFCQSIIESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | fused on N-terminal with |
| | SIEKTISKAKGQPREPQVYTLPPCQEEMTKNQVSLWCLVK | no linker |
| | GFYPSDIAVEWESNGQPENNYKTTPPVLDSDGSFFLYSRL | |
| | TVDKSRWQEGNVFSCSVMHEALHNHYTQKSLSLSLGK | |
| 53 | APTSSSTKKTQLQLEHLLLDLQMILNGINNYKNPKLTRML | W3XX115-T2U3.E44- |
| | TFKFYMPKKATELKHLQCLEEELKPLEEVLNLAQSKNFHL | 26.uIgG4V322, 1st heavy |
| | RPRDLISNINVIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion |
| | REEQFNSTYRVVSVLTVLHQDWLNGKEYKCKVSNKGLPS | and I129L, fused on N- |
| | SIEKTISKAKGQPREPQVYTLPPCQEEMTKNQVSLWCLVK | terminal with no linker |
| | GFYPSDIAVEWESNGQPENNYKTTPPVLDSDGSFFLYSRL | |
| | TVDKSRWQEGNVFSCSVMHEALHNHYTQKSLSLSLGK | |
| 54 | APTSSSTKKTQLQLEHLLLDLQMILNGINNYKNPKLTRML | W3XX115-T2U3.E44- |
| | TVKFYMPKKATELKHLQCLEEELKPLEEVLNLAQSKNFH | 15.uIgG4V322, 1st heavy |
| | LRPRDLISNINVIVLELKGSETTFMCEYADETATIVEFLNR | chain |
| | WITFCQSIIESKYGPPCPPCPAPEAAGGPSVFLFPPKPKDTL | WT IL2 with C-terminal |
| | MISRTPEVTCVVVDVSQEDPEVQFNWYVDGVEVHNAKT | four amino acids deletion |
| | KPREEQFNSTYRVVSVLTVLHQDWLNGKEYKCKVSNKG | and F42I, fused on N- |
| | LPSSIEKTISKAKGQPREPQVYTLPPCQEEMTKNQVSLWCL | terminal with no linker |
| | VKGFYPSDIAVEWESNGQPENNYKTTPPVLDSDGSFFLYS | |
| | RLTVDKSRWQEGNVFSCSVMHEALHNHYTQKSLSLSLGK | |
| 55 | APTSSSTKKTQLQLEHLLLDLQMILNGINNYKNPKLTWML | W3XX115-T2U3.E44- |
| | TFKFYMPKKATELKHLQCLEEELKPLEEVLNLAQSKNFHL | 20.uIgG4V322, 1st heavy |
| | RPRDLISNINVIVLELKGSETTFMCEYADETATIVEFLNRWI | chain |
| | TFCQSIIESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion |
| | REEQFNSTYRVVSVLTVLHQDWLNGKEYKCKVSNKGLPS | and R38W, fused on N- |
| | SIEKTISKAKGQPREPQVYTLPPCQEEMTKNQVSLWCLVK | terminal with no linker |
| | GFYPSDIAVEWESNGQPENNYKTTPPVLDSDGSFFLYSRL | |
| | TVDKSRWQEGNVFSCSVMHEALHNHYTQKSLSLSLGK | |
| 56 | APTSSSTKKTQLQLEHLLLDLQMILNGINNYKNPKLTWML | W3XX115-T2U3.E44- |
| | TFKFYMPKKATELKHLQCLEEELKPLEEVLNLAQSKNFHL | 33.uIgG4V322, 1st heavy |
| | RPRDLISNINVIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | F421 and I129L, fused on |
| | SIEKTISKAKGQPREPQVYTLPPCQEEMTKNQVSLWCLVK | N-terminal with no linker |
| | GFYPSDIAVEWESNGQPENNYKTTPPVLDSDGSFFLYSRL | |
| | TVDKSRWQEGNVFSCSVMHEALHNHYTQKSLSLSLGK | |
| 57 | QVQLVQSGAEVKKPGSSVKVSCKASGFTFTTYYISWVRQ | T2U3.E44 uIgG4V322 |
| | APGQGLEYLGYINMGSGGTNYNEKFKGRVTITADKSTST | format, 2^{nd} heavy chain, |
| | AYMELSSLRSEDTAVYYCAIIGYFDYWGQGTMVTVSSAS | comprising heavy chain of |
| | TKGPSVFPLAPCSRSTSESTAALGCLVKDYFPEPVTVSWNS | a PD-1 binding Fab (U3) |
| | GALTSGVHTFPAVLQSSGLYSLSSVVTVPSSSLGTKTYTCN | |
| | VDHKPSNTKVDKRVESKYGPPCPPCPAPEAAGGPSVFLFP | |
| | PKPKDTLMISRTPEVTCVVVDVSQEDPEVQFNWYVDGVE | |
| | VHNAKTKPREEQFNSTYRVVSVLTVLHQDWLNGKEYKC | |
| | KVSNKGLPSSIEKTISKAKGQPREPQVCTLPPSQEEMTKNQ | |
| | VSLSCAVKGFYPSDIAVEWESNGQPENNYKTTPPVLDSDG | |
| | SFFLVSRLTVDKSRWQEGNVFSCSVMHEALHNHYTQKSL | |
| | SLSLGK | |
| 58 | DVVMTQSPLSLPVTLGQPASISCRSSQSLLDSDGGTYLYW | Light chain for T2U3. E44 |
| | FQQRPGQSPRRLIYLVSTLGSGVPDRFSGSGSGTDFTLKISR | format, comprising light |
| | VEAEDVGVYYCMQLTHWPYTFGQGTKLEIKRTVAAPSVF | chain of a PD-1 binding |
| | IFPPSDEQLKSGTASVVCLLNNFYPREAKVQWKVDNALQS | Fab (U3) |
| | GNSQESVTEQDSKDSTYSLSSTLTLSKADYEKHKVYACEV | |
| | THQGLSSPVTKSFNRGEC | |
| 59 | APTSSSTKKTQLQLEHLLLDLQMILNGINNYKNPKLTRML | W3XX115-T2U10.Z73- |
| | TIKFYMPKKATELKHLQCLEEELKPLEEVLNLAQSKNFHL | 50.uIgG4V322, 1st heavy |
| | RPRDLISNINVIVLELKGSETTFMCEYADRTATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | E110R, F42I and I129L, |
| | SIEKTISKAKGQPREPQVYTLPPCQEEMTKNQVSLWCLVK | fused on N-terminal with |
| | GFYPSDIAVEWESNGQPENNYKTTPPVLDSDGSFFLYSRL | no linker |
| | TVDKSRWQEGNVFSCSVMHEALHNHYTQKSLSLSLGK | |
| 60 | APTSSSTKKTQLQLEHLLLDLQMILNGINNYDNPKLTRML | W3XX115-T2U10.Z73- |
| | TIKFYMPKKATELKHLQCLEEELKPLEEVLNLAQSKNFHL | 51.uIgG4V322, 1st heavy |
| | RPRDLISNINVIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I and I129L, |
| | SIEKTISKAKGQPREPQVYTLPPCQEEMTKNQVSLWCLVK | fused on N-terminal with |
| | GFYPSDIAVEWESNGQPENNYKTTPPVLDSDGSFFLYSRL | no linker |
| | TVDKSRWQEGNVFSCSVMHEALHNHYTQKSLSLSLGK | |
| 61 | APTSSSTKKTQLQLEHLRLDLEMILNGINNYKNPKLTRML | W3XX115-T2U10.Z73- |
| | TIKFYMPKKATELKHLQCLEEELKPLEEVLNLAQSKNFHL | 52.uIgG4V322, 1st heavy |
| | RPRDLISNINVIVLELKGSETTFMCEYADETATIVEFLNRWI | chain |
| | TFCKSIISTLTESKYGPPCPPCPAPEAAGGPSVFLFPPKPKD | WT IL2 with L18R, Q22E, |
| | TLMISRTPEVTCVVVDVSQEDPEVQFNWYVDGVEVHNAK | F42I and Q126K, fused on |
| | TKPREEQFNSTYRVVSVLTVLHQDWLNGKEYKCKVSNK | N-terminal with no linker |
| | GLPSSIEKTISKAKGQPREPQVYTLPPCQEEMTKNQVSLW | |
| | CLVKGFYPSDIAVEWESNGQPENNYKTTPPVLDSDGSFFL | |
| | YSRLTVDKSRWQEGNVFSCSVMHEALHNHYTQKSLSLSL | |
| | GK | |
| 62 | APTSSSTKKTQLQLEHLLHDLQMILNGINNYKNPKLTRML | W3XX115-T2U10.Z73- |
| | TIKFYMPKKATELKHLQCLEEELKPLEEVLNLAQSKNFHL | 53.uIgG4V322, 1st heavy |
| | RPRDLISNINVIVLELKGSETTFMCEYADETATIVEFLNRWI | chain |
| | TFIESIISTLTESKYGPPCPPCPAPEAAGGPSVFLFPPKPKDT | WT IL2 with L19H, F42I, |
| | LMISRTPEVTCVVVDVSQEDPEVQFNWYVDGVEVHNAKT | C125I and Q126E, fused |
| | KPREEQFNSTYRVVSVLTVLHQDWLNGKEYKCKVSNKG | on N-terminal with no |
| | LPSSIEKTISKAKGQPREPQVYTLPPCQEEMTKNQVSLWCL | linker |
| | VKGFYPSDIAVEWESNGQPENNYKTTPPVLDSDGSFFLYS | |
| | RLTVDKSRWQEGNVFSCSVMHEALHNHYTQKSLSLSLGK | |
| 63 | APASSSTKKTQLQLEHLLLNLQMILNGINNYKNPKLTRML | W3XX115-T2U10.Z73- |
| | TIKFYMPKKATELKHLQCLEEELKPLEEVLELAQSKNFHL | 54.uIgG4V322, 1st heavy |
| | RPRDLISNINVIVLELKGSETTFMCEYADETATIVEFLNRWI | chain |
| | TFSQSIISTLTESKYGPPCPPCPAPEAAGGPSVFLFPPKPKDT | WT IL2 with T3A, D20N, |
| | LMISRTPEVTCVVVDVSQEDPEVQFNWYVDGVEVHNAKT | F42I, N71K and C125S, |
| | KPREEQFNSTYRVVSVLTVLHQDWLNGKEYKCKVSNKG | fused on N-terminal with |
| | LPSSIEKTISKAKGQPREPQVYTLPPCQEEMTKNQVSLWCL | no linker |
| | VKGFYPSDIAVEWESNGQPENNYKTTPPVLDSDGSFFLYS | |
| | RLTVDKSRWQEGNVFSCSVMHEALHNHYTQKSLSLSLGK | |
| 64 | APTSSSTKKTQLDLEHLLLDLQMILNGINNYONPKLTRML | W3XX115-T2U10.Z73- |
| | TIKFYMPKKATELKHLQCLEEELKPLEEVLNLAQSKNFHL | 55.uIgG4V322, 1st heavy |
| | RPRDLISNINVIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, Q13D and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 65 | APTSSSTKKTQLQLEHLLLDLQMILNGINNYDNPKLTRML | W3XX115-T2U10.Z73- |
| | TIKFYMPKKATELKHLQCLEEELKPLEEVLNLAQSKNFHL | 56.uIgG4V322, 1st heavy |
| | RPRKLISNINVIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, D84K and |
| | SIEKTISKAKGQPREPQVYTLPPCQEEMTKNQVSLWCLVK | I129L, fuse on |
| | GFYPSDIAVEWESNGQPENNYKTTPPVLDSDGSFFLYSRL | N- |
| | TVDKSRWQEGNVFSCSVMHEALHNHYTQKSLSLSLGK | terminal with no linker |
| 66 | APTSSSTKKTQLQLEHLLLDLQMILNGINNYDNPKLTRML | W3XX115-T2U10.Z73- |
| | TIKFYMPKKATELKHLQCLEEELKPLEEVLNLAQSKNFHL | 57.uIgG4V322, 1st heavy |
| | RPRDLIRNINVIVLELKGSETTFMCEYADETATIVEFLNRW | chain |
| | ITFCQSILESKYGPPCPPCPAPEAAGGPSVFLFPPKPKDTLM | WT IL2 with C-terminal |
| | ISRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, S87R and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 67 | APTSSSTKKTQLQLEHLLLDLQMILNGINNYONPKLTRML | W3XX115-T2U10.Z73- |
| | TIKFYMPKKATELKHLQCLEEELKPLEEVLNLAQSKNFHL | 58.uIgG4V322, 1st heavy |
| | RPRDLISEINVIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, N88K and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 68 | APTSSSTKKTQLQLEHLLLDLQMILNGINNYDNPKLTRML | W3XX115-T2U10.Z73- |
| | TIKFYMPKKATELKHLQCLEEELKPLEEVLNLAQSKNFHL | 59.uIgG4V322, 1st heavy |
| | RPRDLISNINEIVLELKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, V91E and |
| | SIEKTISKAKGQPREPQVYTLPPCQEEMTKNQVSLWCLVK | I129L, fused |
| | GFYPSDIAVEWESNGQPENNYKTTPPVLDSDGSFFLYSRL | on N- |
| | TVDKSRWQEGNVFSCSVMHEALHNHYTQKSLSLSLGK | terminal with no linker |
| 69 | APTSSSTKKTQLQLEHLLLDLQMILNGINNYONPKLTRML | W3XX115-T2U10.Z73- |
| | TIKFYMPKKATELKHLQCLEEELKPLEEVLNLAQSKNFHL | 60.uIgG4V322, 1st heavy |
| | RPRDLISNINVRVLELKGSETTFMCEYADETATIVEFLNRW | chain |
| | ITFCQSILESKYGPPCPPCPAPEAAGGPSVFLFPPKPKDTLM | WT IL2 with C-terminal |
| | ISRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, 192R and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 70 | APTSSSTKKTQLQLEHLLLDLQMILNGINNYDNPKLTRML | W3XX115-T2U10.Z73- |
| | TIKFYMPKKATELKHLQCLEEELKPLEEVLNLAQSKNFHL | 61.uIgG4V322, 1st heavy |
| | RPRDLISNINVDVLELKGSETTFMCEYADETATIVEFLNRW | chain |
| | ITFCQSILESKYGPPCPPCPAPEAAGGPSVFLFPPKPKDTLM | WT IL2 with C-terminal |
| | ISRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, 192D and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 71 | APTSSSTKKTQLQLEHLLLDLQMILNGINNYONPKLTRML | W3XX115-T2U10.Z73- |
| | TIKFYMPKKATELKHLQCLEEELKPLEEVLNLAQSKNFHL | 62.uIgG4V322, 1st heavy |
| | RPRDLISNINVIVDELKGSETTFMCEYADETATIVEFLNRW | chain |
| | ITFCQSILESKYGPPCPPCPAPEAAGGPSVFLFPPKPKDTLM | WT IL2 with C-terminal |
| | ISRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, L94D and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 72 | APTSSSTKKTQLQLEHLLLDLQMILNGINNYONPKLTRML | W3XX115-T2U10.Z73- |
| | TIKFYMPKKATELKHLQCLEEELKPLEEVLNLAQSKNFHL | 63.uIgG4V322, 1st heavy |
| | RPRDLISNINVIVLRLKGSETTFMCEYADETATIVEFLNRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, E95R and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 73 | APTSSSTKKTQLQLEHLLLDLQMILNGINNYDNPKLTRML | W3XX115-T2U10.Z73- |
| | TIKFYMPKKATELKHLQCLEEELKPLEEVLNLAQSKNFHL | 64.uIgG4V322, 1st heavy |
| | RPRDLISNINVIVLELKGSETTFMCEYADETATIVEFLRRWI | chain |
| | TFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, N119R and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 74 | APTSSSTKKTQLQLEHLLLDLQMILNGINNYDNPKLTRML | W3XX115-T2U10.Z73- |
| | TIKFYMPKKATELKHLQCLEEELKPLEEVLNLAQSKNFHL | 65.uIgG4V322, 1st heavy |
| | RPRDLISNINVIVLELKGSETTFMCEYADETATIVEFLNRWI | chain |
| | KFCQSILESKYGPPCPPCPAPEAAGGPSVFLFPPKPKDTLMI | WT IL2 with C-terminal |
| | SRTPEVTCVVVDVSQEDPEVQFNWYVDGVEVHNAKTKP | four amino acids deletion, |
| | REEQFNSTYRVVSVLTVLHQDWLNGKEYKCKVSNKGLPS | K32D, F42I, T123K and |
| | SIEKTISKAKGQPREPQVYTLPPCQEEMTKNQVSLWCLVK | I129L, fused on N- |
| | GFYPSDIAVEWESNGQPENNYKTTPPVLDSDGSFFLYSRL | terminal with no linker |
| | TVDKSRWQEGNVFSCSVMHEALHNHYTQKSLSLSLGK | |
| 75 | QVQLVESGGGVVQPGGSLRLSCAASDSIDSLVNMGWYRQ | Z73 uIgG4V322 format, |
| | APGKQRELVALIATYITHYADFVKGRFTISRDNSKNTLYL | 2nd heavy chain, |
| | QMNSLRAEDTAVYYCYARNIIVDYWGQGTLVTVSSGGG | comprising a VHH |
| | GSGGGGSGGGGSQVQLVESGGGVVQPGGSLRLSCAASDS | |
| | IDSLVNMGWYRQAPGKQRELVALIATYITHYADFVKGRF | |
| | TISRDNSKNTLYLQMNSLRAEDTAVYYCYARNIIVDYWG | |
| | QGTLVTVSSESKYGPPCPPCPAPEAAGGPSVFLFPPKPKDT | |
| | LMISRTPEVTCVVVDVSQEDPEVQFNWYVDGVEVHNAKT | |
| | KPREEQFNSTYRVVSVLTVLHQDWLNGKEYKCKVSNKG | |
| | LPSSIEKTISKAKGQPREPQVCTLPPSQEEMTKNQVSLSCA | |
| | VKGFYPSDIAVEWESNGQPENNYKTTPPVLDSDGSFFLVS | |
| | RLTVDKSRWQEGNVFSCSVMHEALHNHYTQKSLSLSLG | |
| 76 | APTSSSTKKTQLQLEHLRLDLEMILNGINNYKNPKLTRML | W3XX115-T2U10.Z73- |
| | TFKFYMPKKATELKHLQCLEEELKPLEEVLNLAQSKNFHL | 66.uIgG4V322, 1st heavy |
| | RPRDLISNINVIVLELKGSETTFMCEYADETATIVEFLNRWI | chain |
| | TFCKSIISTLTESKYGPPCPPCPAPEAAGGPSVFLFPPKPKD | WT IL2 with L18R, Q22E |
| | TLMISRTPEVTCVVVDVSQEDPEVQFNWYVDGVEVHNAK | and Q126K, fused on N- |
| | TKPREEQFNSTYRVVSVLTVLHQDWLNGKEYKCKVSNK | terminal with no linker |
| | GLPSSIEKTISKAKGQPREPQVYTLPPCQEEMTKNQVSLW | |
| | CLVKGFYPSDIAVEWESNGQPENNYKTTPPVLDSDGSFFL | |
| | YSRLTVDKSRWQEGNVFSCSVMHEALHNHYTQKSLSLSL | |
| | GK | |
| 77 | APTSSSTKKTQLQLEHLLHDLQMILNGINNYKNPKLTRML | W3XX115-T2U10.Z73- |
| | TFKFYMPKKATELKHLQCLEEELKPLEEVLNLAQSKNFHL | 67.uIgG4V322, 1st heavy |
| | RPRDLISNINVIVLELKGSETTFMCEYADETATIVEFLNRWI | chain |
| | TFIESIISTLTESKYGPPCPPCPAPEAAGGPSVFLFPPKPKDT | WT IL2 with L19H, C125I |
| | LMISRTPEVTCVVVDVSQEDPEVQFNWYVDGVEVHNAKT | and Q126E, fused on N- |
| | KPREEQFNSTYRVVSVLTVLHQDWLNGKEYKCKVSNKG | terminal with no linker |
| | LPSSIEKTISKAKGQPREPQVYTLPPCQEEMTKNQVSLWCL | |
| | VKGFYPSDIAVEWESNGQPENNYKTTPPVLDSDGSFFLYS | |
| | RLTVDKSRWQEGNVFSCSVMHEALHNHYTQKSLSLSLGK | |
| 78 | APASSSTKKTQLQLEHLLLNLQMILNGINNYKNPKLTRML | W3XX115-T2U10.Z73- |
| | TEKFYMPKKATELKHLQCLEEELKPLEEVLKLAQSKNFHL | 68.uIgG4V322, 1st heavy |
| | RPRDLISNINVIVLELKGSETTFMCEYADETATIVEFLNRWI | chain |
| | TFSQSIISTLTESKYGPPCPPCPAPEAAGGPSVFLFPPKPKDT | WT IL2 with T3A, D20N, |
| | LMISRTPEVTCVVVDVSQEDPEVQFNWYVDGVEVHNAKT | N71K and C125S, fused on |
| | KPREEQFNSTYRVVSVLTVLHQDWLNGKEYKCKVSNKG | N-terminal with no linker |
| | LPSSIEKTISKAKGQPREPQVYTLPPCQEEMTKNQVSLWCL | |
| | VKGFYPSDIAVEWESNGQPENNYKTTPPVLDSDGSFFLYS | |
| | RLTVDKSRWQEGNVFSCSVMHEALHNHYTQKSLSLSLGK | |
| 104 | APTSSSTKKTQLRLEHLLLDLQMILNGINNYDNPKLTRML | W3xx115-T2U10.Z73- |
| | TIKFYMPKKATELKHLQCLEEELKPLEEVLNLAQSKNFHL | 69.uIgG4V322, 1st heavy |
| | RPRDLISNINVIVLELKGSETTFMCEYADETATIVEFLNRWI | chain |
| | TFCQSILESKYGPPCPPCPAPEAAGGPSVFLFPPKPKDTLMI | WT IL2 with C terminal |
| | SRTPEVTCVVVDVSQEDPEVQFNWYVDGVEVHNAKTKP | truncation of 4 amino |
| | REEQFNSTYRVVSVLTVLHQDWLNGKEYKCKVSNKGLPS | acids, Q13R, K32D, F42I |
| | SIEKTISKAKGQPREPQVYTLPPCQEEMTKNQVSLWCLVK | and I129L substitutions |
| | GFYPSDIAVEWESNGQPENNYKTTPPVLDSDGSFFLYSRL | |
| | TVDKSRWQEGNVFSCSVMHEALHNHYTQKSLSLSLGK | |
| 105 | APTSSSTKKTQLQLEHLLLDLQMILNGINNYDNPKLTRML | W3xx115-T2U10.Z73- |
| | TIKFYMPKKATELKHLQCLEEELKPLEEVLNLAQSKNFHL | 70.uIgG4V322, 1st heavy |
| | RPRTLISNINVIVLELKGSETTFMCEYADETATIVEFLNRWI | chain |
| | TFCQSILESKYGPPCPPCPAPEAAGGPSVFLFPPKPKDTLMI | WT IL2 with C terminal |
| | SRTPEVTCVVVDVSQEDPEVQFNWYVDGVEVHNAKTKP | truncation of 4 amino |
| | REEQFNSTYRVVSVLTVLHQDWLNGKEYKCKVSNKGLPS | acids, D84T, K32D, F42I |
| | SIEKTISKAKGQPREPQVYTLPPCQEEMTKNQVSLWCLVK | and I129L substitutions |
| | GFYPSDIAVEWESNGQPENNYKTTPPVLDSDGSFFLYSRL | |
| | TVDKSRWQEGNVFSCSVMHEALHNHYTQKSLSLSLG | |
| 106 | APTSSSTKKTQLQLEHLLLDLQMILNGINNYDNPKLTRML | W3xx115-T2U10.Z73- |
| | TIKFYMPKKATELKHLQCLEEELKPLEEVLNLAQSKNFHL | 71.uIgG4V322, 1st heavy |
| | RPRDLIININVIVLELKGSETTFMCEYADETATIVEFLNRWI | chain |
| | TFCQSILESKYGPPCPPCPAPEAAGGPSVFLFPPKPKDTLMI | WT IL2 with C terminal |
| | SRTPEVTCVVVDVSQEDPEVQFNWYVDGVEVHNAKTKP | truncation of 4 amino |
| | REEQFNSTYRVVSVLTVLHQDWLNGKEYKCKVSNKGLPS | acids, S87I, K32D, F42I |
| | SIEKTISKAKGQPREPQVYTLPPCQEEMTKNQVSLWCLVK | and I129L substitutions |
| | GFYPSDIAVEWESNGQPENNYKTTPPVLDSDGSFFLYSRL | |
| | TVDKSRWQEGNVFSCSVMHEALHNHYTQKSLSLSLGK | |
| 107 | APTSSSTKKTQLQLEHLLLDLQMILNGINNYDNPKLTRML | W3xx115-T2U10.Z73- |
| | TIKFYMPKKATELKHLQCLEEELKPLEEVLNLAQSKNFHL | 72.uIgG4V322, 1st heavy |
| | RPRDLISNINSIVLELKGSETTFMCEYADETATIVEFLNRWI | chain |
| | TFCQSILESKYGPPCPPCPAPEAAGGPSVFLFPPKPKDTLMI | WT IL2 with C terminal |
| | SRTPEVTCVVVDVSQEDPEVQFNWYVDGVEVHNAKTKP | truncation of 4 amino |
| | REEQFNSTYRVVSVLTVLHQDWLNGKEYKCKVSNKGLPS | acids, V91S, K32D, F42I |
| | SIEKTISKAKGQPREPQVYTLPPCQEEMTKNQVSLWCLVK | and I129L substitutions |
| | GFYPSDIAVEWESNGQPENNYKTTPPVLDSDGSFFLYSRL | |
| | TVDKSRWQEGNVFSCSVMHEALHNHYTQKSLSLSLGK | |
| 108 | APTSSSTKKTQLQLEHLLLDLQMILNGINNYDNPKLTRML | W3xx115-T2U10.Z73- |
| | TIKFYMPKKATELKHLQCLEEELKPLEEVLNLAQSKNFHL | 73.uIgG4V322, 1st heavy |
| | RPRDLISNINVIVLYLKGSETTFMCEYADETATIVEFLNRW | chain |
| | ITFCQSILESKYGPPCPPCPAPEAAGGPSVFLFPPKPKDTLM | WT IL2 with C terminal |
| | ISRTPEVTCVVVDVSQEDPEVQFNWYVDGVEVHNAKTKP | truncation of 4 amino |
| | REEQFNSTYRVVSVLTVLHQDWLNGKEYKCKVSNKGLPS | acids, E95Y, K32D, F42I |
| | SIEKTISKAKGQPREPQVYTLPPCQEEMTKNQVSLWCLVK | and I129L substitutions |
| | GFYPSDIAVEWESNGQPENNYKTTPPVLDSDGSFFLYSRL | |
| | TVDKSRWQEGNVFSCSVMHEALHNHYTQKSLSLSLGK | |
| 109 | APTSSSTKKTQLQLEHLLLDLQMILNGINNYDNPKLTRML | W3xx115-T2U10.Z73- |
| | TIKFYMPKKATELKHLQCLEEELKPLEEVLNLAQSKNFHL | 74.uIgG4V322, 1st heavy |
| | RPRDLISNINVIVLELKGSETTFMCEYADETATIVEFLQRWI | chain |
| | TFCQSILESKYGPPCPPCPAPEAAGGPSVFLFPPKPKDTLMI | WT IL2 with C terminal |
| | SRTPEVTCVVVDVSQEDPEVQFNWYVDGVEVHNAKTKP | truncation of 4 amino |
| | REEQFNSTYRVVSVLTVLHQDWLNGKEYKCKVSNKGLPS | acids, N119Q, K32D, F42I |
| | SIEKTISKAKGQPREPQVYTLPPCQEEMTKNQVSLWCLVK | and I129L substitutions |
| | GFYPSDIAVEWESNGQPENNYKTTPPVLDSDGSFFLYSRL | |
| | TVDKSRWQEGNVFSCSVMHEALHNHYTQKSLSLSLGK | |
| 110 | APTSSSTKKTQLQLEHLLLDLQMILNGINNYDNPKLTRML | W3xx115-T2U10.Z73- |
| | TIKFYMPKKATELKHLQCLEEELKPLEEVLNLAQSKNFHL | 75.uIgG4V322, 1st heavy |
| | RPRDLISNINVIVLELKGSETTFMCEYADETATIVEFLNRWI | chain |
| | VFCQSILESKYGPPCPPCPAPEAAGGPSVFLFPPKPKDTLMI | WT IL2 with C terminal |
| | SRTPEVTCVVVDVSQEDPEVQFNWYVDGVEVHNAKTKP | truncation of 4 amino |
| | REEQFNSTYRVVSVLTVLHQDWLNGKEYKCKVSNKGLPS | acids, T123V, K32D, F42I |
| | SIEKTISKAKGQPREPQVYTLPPCQEEMTKNQVSLWCLVK | and I129L substitutions |
| | GFYPSDIAVEWESNGQPENNYKTTPPVLDSDGSFFLYSRL | |
| | TVDKSRWQEGNVFSCSVMHEALHNHYTQKSLSLSLGK | |

TABLE D
Amino acid sequences of IL2R proteins

| | | |
|---|---|---|
| 35 | ELCDDDPPEIPHATFKAMAYKEGTMLNCECKRGFRRIKSG | W3XX115- |
| | SLYMLCTGNSSHSSWDNQCQCTSSATRNTTKQVTPQPEE | hPro1.ECD.His, IL2Rα |
| | QKERKTTEMQSPMQPVDQASLPGHCREPPPWENEATERI | ECD |
| | YHFVVGQMVYYQCVQGYRALHRGPAESVCKMTHGKTR | |
| | WTQPQLICTGEMETSQFPGEEKPQASPEGRPESETSCHHH | |
| | HHH | |
| 36 | AVNGTSQFTCFYNSRANISCVWSQDGALQDTSCQVHAWP | W369-hPro2.ECD.His, |
| | DRRRWNQTCELLPVSQASWACNLILGAPDSQKLTTVDIVT | IL2Rβ ECD |
| | LRVLCREGVRWRVMAIQDFKPFENLRLMAPISLQVVHVE | |
| | THRCNISWEISQASHYFERHLEFEARTLSPGHTWEEAPLLT | |
| | LKQKQEWICLETLTPDTQYEFQVRVKPLQGEFTTWSPWS | |
| | QPLAFRTKPAALGKDLEGGGGSGGGGHHHHHH | |
| 37 | LNTTILTPNGNEDTTADFFLTTMPTDSLSVSTLPLPEVQCF | W369-hPro1.ECD.His, IL- |
| | VFNVEYMNCTWNSSSEPQPTNLTLHYWYKNSDNDKVQK | 2Rγ ECD |
| | CSHYLFSEEITSGCQLQKKEIHLYQTFVVQLQDPREPRRQA | |
| | TQMLKLQNLVIPWAPENLTLHKLSESQLELNWNNRFLNH | |
| | CLEHLVQYRTDWDHSWTEQSVDYRHKFSLPSVDGQKRY | |
| | TFRVRSRFNPLCGSAQHWSEWSHPIHWGSNTSKENLEGG | |
| | GGSGGGGHHHHHH | |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, IL-2 Variants and Fusion Proteins

### 1.1 Preparation of Materials

Information on the commercially available materials used in the Examples is provided in Table 1.

TABLE 1

| Materials | Vendor | Cat |
|---|---|---|
| ExpiFectamine ™ 293 | Invitrogen | A14525 |
| Transfection Kit | | |
| Opti-MEM | Invitrogen | 31985070 |
| Expi293 Expression Medium | Invitrogen | A1435101 |
| ExpiFectamine ™ 293 cell | Invitrogen | A14635 |
| Ni column | GE healthcare | 173712 |
| Protein A column | GE healthcare | 175438 |
| HPLC-SEC | TOSOH | 0008541 |
| NuPAGE4%-12% Bis-Tris Gel | Thermo Fisher | NP0322BOX |
| ExpiFectamine ™ 293 | Invitrogen | A14525 |
| Transfection Kit | | |
| Opti-MEM | Invitrogen | 31985070 |
| Expi293 Expression Medium | Invitrogen | A1435101 |
| HH cell | ATCC | CRL2105 |
| NK92 cell | ATCC | CRL2105 |
| PE-Anti Human CD4 | Invitrogen | 12-0049-42 |
| FITC-Anti Human CD3 | Bio legend | 317306.00 |
| APC-Cy7 Mouse Anti-Human | BD Biosciences | Pharmingen- |
| CD8 | | 557834 |
| Alexa Fluor 647 mouse anti-Stat5 | BD Biosciences | 562076 |
| (pY694) | | |
| APC-Anti Human Foxp3 | BioLegend | 17-4776-42 |
| PE-Anti Human CD25 clone | BioLegend | 302606 |
| BC96 | | |
| BD Phosflow ™ Fix Buffer I | BD Biosciences | BDB557870 |
| Phosflow Perm Buffer III | BD Biosciences | BDB558050 |
| Treg expansion kit, human | Miltenyi | 130-095-345 |
| Easy Sep Human CD4 + CD127 low | Stemcell | 18063 |
| CD25+ Regulatory T cell | | |
| isolation Kit | | |
| Recombinant Human Interleukin-2 | Si Huan Sheng | |
| Injecition | Wu | |
| Goat anti-human IgG Fc R-PE | Jackson | 109-115-098 |
| EasySep. Human CD8+ T Cell | Stemcell | 17953 |
| Isolation Kit | | |
| BV510 anti-mouse CD45, 30-F11 | BioLegend | 103138 |
| BV786 anti-mouse CD3, 500A1 | BD Biosciences | 740854 |
| BV421 anti-mouse CD4, RM4-4 | BD Biosciences | 740008 |
| BUV661 anti-mouse CD8, 53-6.7 | BD Biosciences | 740854 |
| PE-Cy7 anti-mouse CD25, PC61 | BioLegend | 102016 |
| BV650 anti-mouse CD44, IM7 | BD Biosciences | 740455 |
| BUV395 anti-mouse CD62L, | BD Biosciences | 740218 |
| MEL-14 | | |
| PE-Cyanine5 anti-mouse NK1.1 | BioLegend | 103138 |
| AF700 anti-Ki67, 16A8 | BioLegend | 652420 |
| PE anti-mouse Foxp3, MF-14 | BioLegend | 126404 |
| FITC anti-mouse GranzymeB, GB11 | BioLegend | 515403 |
| APC anti-mouse Tbet, 4B10 | BioLegend | 644814 |

### 1.2 Generation of IL-2/Fc Polypeptide Complexes Comprising Wild Type IL-2 or IL-2 Variants Construction of Plasmids for Expression of IL-2/Fc Polypeptide Complexes

THE STRUCTURES OF THE IL-2/FC POLYPEPTIDE COMPLEXES ARE AS SHOWN IN FIG. 1. THE E44 FORMAT INDICATES ONE IL-2 DOMAIN IS OPERABLY LINKED AT THE N TERMINAL OF ONE CHAIN OF THE FC REGION, AND A FAB IS OPERABLY LINKED TO THE N TERMINAL OF THE OTHER CHAIN OF THE FC REGION. THE Z20 FORMAT INDICATES THAT TWO IL-2 DOMAINS ARE EACH OPERABLY LINKED AT THE N TERMINAL OF ONE CHAIN OF THE FC REGION. THE Z73 FORMAT INDICATES ONE IL-2 DOMAIN IS OPERABLY LINKED AT THE N TERMINAL OF ONE CHAIN OF THE FC REGION, AND TWO VHHS ARE OPERABLY LINKED TO THE N TERMINAL OF THE OTHER CHAIN OF THE FC REGION.

Polynucleotides encoding the VL, VH, Ck, CH1 and VHH of antigen-binding moieties derived from antibodies, were respectively amplified by PCR from DNA templates. Polynucleotides encoding wild type IL-2 and IL-2 variants were synthesized by Sangon Biotech Inc. Polynucleotides encoding native light chain sequences of the antibodies were inserted into a linearized vector containing a CMV promoter and a kappa or lambda signal peptide. The DNA fragments of anti-target VH-CH1 and wild-type IL-2 or IL-2 variants were inserted into a linearized vector, which contains the constant region CH2-CH3 of human IgG1 or IgG4 with or without a (G4S) n linker, or hinge truncated IgG4 according to the formats. The vector contains a CMV promoter and a human antibody heavy chain signal peptide.

Benchmark antibodies: W3xx115-BMK7, W3xx115-BMK8, W3xx115-BMK14 are benchmark polypeptide complexes in E44 format comprising specific IL-2 variants. W3xx115-BMK7 comprises an IL-2 variant with 101, 141& 169 amino acids deletion and L53R+Q162H substitutions (SyntheKine). W3xx115-BMK8 comprises an IL-2 variant with L18R, Q22E and Q126K substitutions (SyntheKine). W3xx115-BMK14 comprises an IL-2 variant with N88D substitution. W327199-BMK1 and W327199-BMK2 are benchmark polypeptide complexes in F114 format comprising IL-15 instead of IL-2 domains.

THE GENERATED IL-2/FC POLYPEPTIDE COMPLEXES AND THEIR CORRESPONDING IL-2 FORMAT (WT OR VARIANT) ARE LISTED IN TABLE 2. THE ID OR DESIGNATION OF THE IL-2/FC POLYPEPTIDE COMPLEXES INDICATE THEIR STRUCTURE.

TABLE 2
IL-2/Fc polypeptide complexes

| | | Linkage between | Antigen- |
|---|---|---|---|
| | IL-2 moiety | IL-2 moiety and | binding |
| Protein ID | (compared to WT IL-2) | Fc | moiety |
| W3xx115-T2U0.E44-6.uIgG4V322 | C terminal truncation: 4 amino | no linker | Fab |
| | acids (SEQ ID NO: 2) | | |
| W3xx115-T2U0.E44-26.uIgG4V322 | C terminal truncation of 4 amino | no linker | Fab |
| | acids and I129L substitution | | |
| | (SEQ ID NO: 3) | | |
| W3xx115-T2U0.E44-40.uIgG4V322 | C terminal truncation of 4 amino | no linker | Fab |
| | acids and E110R substitution | | |
| | (SEQ ID NO: 4) | | |
| W3xx115-T2U0.E44-41.uIgG4V322 | C terminal truncation of 4 amino | no linker | Fab |
| | acids and I122Y substitution | | |
| | (SEQ ID NO: 5) | | |
| W3xx115-T2U0.E44-42.uIgG4V322 | C terminal truncation of 4 amino | no linker | Fab |
| | acids and I28P substitution | | |
| | (SEQ ID NO: 6) | | |
| W3xx115-T2U0.E44-43.uIgG4V322 | C terminal truncation of 4 amino | no linker | Fab |
| | acids and K32D substitution | | |
| | (SEQ ID NO: 7) | | |
| W3xx115-T2U0.E44-44.uIgG4V322 | C terminal truncation of 4 amino | no linker | Fab |
| | acids and E52G substitution | | |
| | (SEQ ID NO: 8) | | |
| W3xx115-T2U0.E44-45.uIgG4V322 | C terminal truncation of 4 amino | no linker | Fab |
| | acids and K76R substitution | | |
| | (SEQ ID NO: 9) | | |
| W3xx115-T2U0.E44-46.uIgG4V322 | C terminal truncation of 4 amino | no linker | Fab |
| | acids and F78G substitution | | |
| | (SEQ ID NO: 10) | | |
| W3xx115-T2U0.E44-47.uIgG4V322 | C terminal truncation of 4 amino | no linker | Fab |
| | acids and P82Y substitution | | |
| | (SEQ ID NO: 11) | | |
| W3xx115-T2U0.E44-48.uIgG4V322 | C terminal truncation of 4 amino | no linker | Fab |
| | acids, E110R and I129L | | |
| | substitution (SEQ ID NO: 12) | | |
| W3xx115-T2U0.E44-49.uIgG4V322 | C terminal truncation of 4 amino | no linker | Fab |
| | acids, K32D and I129L | | |
| | substitution (SEQ ID NO: 13) | | |
| W3xx115-T2U0.E44-1.uIgG4V322 | WT (SEQ ID NO: 1) | no linker | Fab |
| W3xx115-T3U0.E44-1.uIgG4V322 | F42V/Y45A/L72G | no linker | Fab |
| | (SEQ ID NO: 14) | | |
| W3xx115-T2.Z20-1.uIgG4V322 | C terminal truncation: 4 amino | no linker | None |
| | acids, bivalent (SEQ ID NO: 2) | | |
| W3xx115-T2.Z20-2.uIgG4V322 | C terminal truncation of 4 amino | no linker | None |
| | acids and I129L substitution, | | |
| | bivalent (SEQ ID NO: 3) | | |
| W3xx115-T2.Z20-4.uIgG4V322 | C terminal truncation of 4 amino | no linker | None |
| | acids, E110R and I129L | | |
| | substitution, bivalent | | |
| | (SEQ ID NO: 12) | | |
| W3xx115-T2.Z20-5.uIgG4V322 | C terminal truncation of 4 amino | no linker | None |
| | acids, K32D and I129L | | |
| | substitution, bivalent | | |
| | (SEQ ID NO: 13) | | |
| W3xx115-BMK7 | Mouse surrogate of BMK8 | no linker | Fab |
| | (SyntheKine) | | |
| W3xx115-BMK8 | L18R, Q22E, Q126K | no linker | Fab |
| | (SyntheKine) | | |
| W3xx115-BMK14 | N88D | no linker | Fab |
| W3XX115-U3T3.F114-1.uIgG4V322 | F42V/Y45A/L72G | (G4S)2 | Fab |
| W3xx115-T2U3.E44-6.uIgG4V322 | C terminal truncation of 4 amino | no linker | Fab |
| | acids (SEQ ID NO: 2) | | |
| W3xx115-T2U3.E44-26.uIgG4V322 | C terminal truncation of 4 amino | no linker | Fab |
| | acids and I129L substitution | | |
| | (SEQ ID NO: 3) | | |
| W3xx115-T2U3.E44-15.uIgG4V322 | C terminal truncation of 4 amino | no linker | Fab |
| | acids and F42I substitution | | |
| | (SEQ ID NO: 79) | | |
| W3xx115-T2U3.E44-20.uIgG4V322 | C terminal truncation of 4 amino | no linker | Fab |
| | acids and R38W substitution | | |
| | (SEQ ID NO: 111) | | |
| W3xx115-T2U3.E44-33.uIgG4V322 | C terminal truncation of 4 amino | no linker | Fab |
| | acids, F42I and I129L | | |
| | substitution (SEQ ID NO: 80) | | |
| W3xx115-T2U10.Z73-50.uIgG4V322 | C terminal truncation of 4 amino | no linker | VHH |
| | acids, F42I, E110R and I129L | | |
| | substitution (SEQ ID NO: 81) | | |
| W3xx115-T2U10.Z73-51.uIgG4V322 | C terminal truncation of 4 amino | no linker | VHH |
| | acids, K32D, F42I and I129L | | |
| | substitution (SEQ ID NO: 82) | | |
| W3xx115-T2U10.Z73-52.uIgG4V322 | L18R/Q22E/F42I/Q126K | no linker | VHH |
| | (SEQ ID NO: 83) | | |
| W3xx115-T2U10.Z73-53.uIgG4V322 | L19H/F42I/C125I/Q126E | no linker | VHH |
| | (SEQ ID NO: 84) | | |
| W3xx115-T2U10.Z73-54.uIgG4V322 | T3A/D20N/F42I/N71K/C125S | no linker | VHH |
| | (SEQ ID NO: 85) | | |
| W3xx115-T2U10.Z73-55.uIgG4V322 | C terminal truncation of 4 amino | no linker | VHH |
| | acids, Q13D, K32D, F42I and | | |
| | I129L substitution | | |
| | (SEQ ID NO: 86) | | |
| W3xx115-T2U10.Z73-56.uIgG4V322 | C terminal truncation of 4 amino | no linker | VHH |
| | acids, D84K, K32D, F42I and | | |
| | I129L substitution | | |
| | (SEQ ID NO: 87) | | |
| W3xx115-T2U10.Z73-57.uIgG4V322 | C terminal truncation of 4 amino | no linker | VHH |
| | acids, S87R, K32D, F42I and | | |
| | I129L substitution | | |
| | (SEQ ID NO: 88) | | |
| W3xx115-T2U10.Z73-58.uIgG4V322 | C terminal truncation of 4 amino | no linker | VHH |
| | acids, N88K, K32D, F42I and | | |
| | I129L substitution | | |
| | (SEQ ID NO: 89) | | |
| W3xx115-T2U10.Z73-59.uIgG4V322 | C terminal truncation of 4 amino | no linker | VHH |
| | acids, V91E, K32D, F42I and | | |
| | I129L substitution | | |
| | (SEQ ID NO: 90) | | |
| W3xx115-T2U10.Z73-60.uIgG4V322 | C terminal truncation of 4 amino | no linker | VHH |
| | acids, 192R, K32D, F42I and | | |
| | I129L substitution | | |
| | (SEQ ID NO: 91) | | |
| W3xx115-T2U10.Z73-61.uIgG4V322 | C terminal truncation of 4 amino | no linker | VHH |
| | acids, 192D, K32D, F42I and | | |
| | I129L substitution | | |
| | (SEQ ID NO: 92) | | |
| W3xx115-T2U10.Z73-62.uIgG4V322 | C terminal truncation of 4 amino | no linker | VHH |
| | acids, L94D, K32D, F42I and | | |
| | I129L substitution | | |
| | (SEQ ID NO: 93) | | |
| W3xx115-T2U10.Z73-63.uIgG4V322 | C terminal truncation of 4 amino | no linker | VHH |
| | acids, E95R, K32D, F42I and | | |
| | I129L substitution | | |
| | (SEQ ID NO: 94) | | |
| W3xx115-T2U10.Z73-64.uIgG4V322 | C terminal truncation of 4 amino | no linker | VHH |
| | acids, N119R, K32D, F42I and | | |
| | I129L substitution | | |
| | (SEQ ID NO: 95) | | |
| W3xx115-T2U10.Z73-65.uIgG4V322 | C terminal truncation of 4 amino | no linker | VHH |
| | acids, T123K, K32D, F42I and | | |
| | I129L substitution | | |
| | (SEQ ID NO: 96) | | |
| W3xx115-T2U10.Z73-66.uIgG4V322 | L18R/Q22E/Q126K | no linker | VHH |
| | (SEQ ID NO: 112) | | |
| W3xx115-T2U10.Z73-67.uIgG4V322 | L19H/C125I/Q126E | no linker | VHH |
| | (SEQ ID NO: 113) | | |
| W3xx115-T2U10.Z73-68.uIgG4V322 | T3A/D20N/N71K/C125S | no linker | VHH |
| | (SEQ ID NO: 114) | | |
| W3xx115-T2U10.Z73-69.uIgG4V322 | C terminal truncation of 4 amino | no linker | VHH |
| | acids, Q13R, K32D, F42I and | | |
| | I129L substitution | | |
| | (SEQ ID NO: 97) | | |
| W3xx115-T2U10.Z73-70.uIgG4V322 | C terminal truncation of 4 amino | no linker | VHH |
| | acids, D84T, K32D, F42I and | | |
| | I129L substitution | | |
| | (SEQ ID NO: 98) | | |
| W3xx115-T2U10.Z73-71.uIgG4V322 | C terminal truncation of 4 amino | no linker | VHH |
| | acids, S87I, K32D, F42I and | | |
| | I129L substitution | | |
| | (SEQ ID NO: 99) | | |
| W3xx115-T2U10.Z73-72.uIgG4V322 | C terminal truncation of 4 amino | no linker | VHH |
| | acids, V91S, K32D, F42I and | | |
| | I129L substitution | | |
| | (SEQ ID NO: 100) | | |
| W3xx115-T2U10.Z73-73.uIgG4V322 | C terminal truncation of 4 amino | no linker | VHH |
| | acids, E95Y, K32D, F42I and | | |
| | I129L substitution | | |
| | (SEQ ID NO: 101) | | |
| W3xx115-T2U10.Z73-74.uIgG4V322 | C terminal truncation of 4 amino | no linker | VHH |
| | acids, N119Q, K32D, F42I and | | |
| | I129L substitution | | |
| | (SEQ ID NO: 102) | | |
| W3xx115-T2U10.Z73-75.uIgG4V322 | C terminal truncation of 4 amino | no linker | VHH |
| | acids, T123V, K32D, F42I and | | |
| | I129L substitution | | |
| | (SEQ ID NO: 103) | | |

### Generation of Il-2/Fc Polypeptide Complexes in Expi293 Cells

Expi293 cells (Thermofisher, A14635) or ExpiCHO cells (Thermofisher, A29133) were prepared for protein expression, and diluted with pre-warmed Expi293 Expression Medium. The transfection reagents were composed of A and B. Where, the regent A was prepared by adding plasmids into pre-warmed Opti-MEM, and reagent B was prepared by adding transfection reagent to Opti-MEM as well. Then reagent A and B were mixed gently and allowed to incubate for 20 minutes at room temperature. For the transfection procedure, the mixture above was added to cells followed by incubation on shaker in 37° C., 8% CO2, and rotating at 120 rpm for 18-20 hours. After transfection, Enhancer 1 and Enhancer 2 were added to the medium with culture for another 5 days to harvest supernatant.

### Purification of Il-2/Fc Polypeptide Complexes

The supernatant of Expi293 cells or ExpiCHO cells as described above were collected and filtered for purification using Protein A column (GE Healthcare, Cat. 175438) or Protein G column (GE Healthcare, Cat. 170618). The concentration of purified Fc-polypeptide complexes was determined by absorbance at 280 nm. The molecular weight and purity were tested by SDS-PAGE and SEC-HPLC, respectively.

The Fc polypeptide complexes of IL-2 and its variants were generated with purity over 90%, indicating they are intact and well-assembled molecules under physiological condition.

### 1.3 Generation of IL-2Rα, IL-2Rβ and IL-2Rγ

Polynucleotides encoding extracellular domains of IL2Rα (CD25, SEQ ID NO: 135) and IL2Rβ (CD122, SEQ ID NO: 136) and IL2Rγ (CD132, SEQ ID NO: 137), with 6×His tag on C-terminal, were synthesized by Sangon Biotech Inc. The vector with CMV promoter, were then transfected into Expi293 cells. The supernatant of transfected Expi293 cells was collected as described above and filtered for purification using Ni-column (GE Healthcare, Cat. 173712). The concentration of purified His-tagged proteins was determined by absorbance at 280 nm, and molecular weight and purity were tested by SDS-PAGE and SEC-HPLC, respectively.

### Example 2

### In Vitro Characterization of IL-2 Variants

2.1 pSTAT5 Activation Assay of Human CD8+ T Cells

STAT5 is a downstream signal maker strictly associated with T cell activation. Human resting CD8+ T in PBMC were analyzed for STAT5 phosphorylation following 30 minutes' incubation with IL-2 polypeptide complexes comprising WT IL-2 or IL-2 variants. At the end of the treatment, PBMC were immediately fixed by BD Phosflow Fix Buffer I and then incubated with pre-chilled BD Phosflow Perm Buffer III. After incubation and fixation, cells were stained with anti-CD3, anti-CD4 and anti-CD8 antibodies for 30 minutes at room temperature. After that, cells were treated with Perm Buffer III for permeabilization, and allowed for staining with anti-pSTAT5 antibody for 30 minutes. Lymphocytes were first gated on the basis of SSC and FSC, and then gated based on CD3 followed by CD4 and CD8 expression to identify CD8+ T cells. Finally, the phosphorylation level of STAT5 in the CD8+ T populations was determined.

Primary human CD8+ T (expressing intermediate affinity receptor, IL-2Rβ/γc) were activated by IL-2 polypeptide complexes comprising IL-2 variants, which is reflected as STAT5 phosphorylation in different degree (FIG. 2, Table 3). T2U0.E44-46 showed 7.6-fold potency reduction relative to T2U0.E44-1 (WT IL-2), followed by T2U0.E44-44 (11-fold), T2U0.E44-43 (17-fold), T2U0.E44-6 (18-fold), T2U0.E44-47 (27-fold), T2U0.E44-45 (31-fold) and T2U0.E44-40 (37-fold). The efficacy (maximum MFI) of T2U0.E44-40 induced activation was weaker than others. Truncation and together with substitutions decreased IL-2 affinity to IL-2Rβ/γc complex, and stabilization design did not disturb in vitro potency of IL-2 variants.

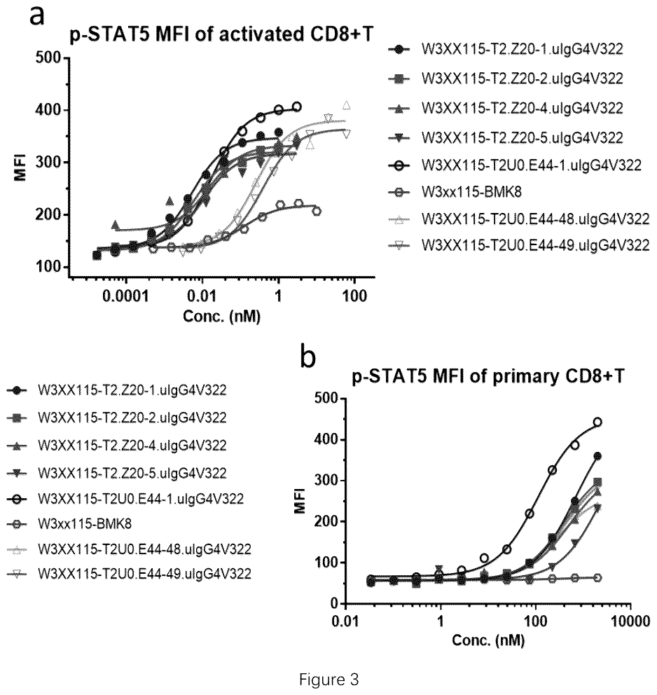

E110R or K32D was combined with truncation and I129L into IL-2 muteins to further attenuate potency to IL-2Rβ/γc complex. As shown in Table 4 and FIG. 3b, IL-2 variants of T2U0.E44-48 and T2U0.E44-49 showed comparable attenuation of 2.7 and 3.8 folds to W3XX115-T2U0.E44-1.uIgG4V322.

TABLE 3
Summarized potency of IL-2 variants in primary CD8+ T

| | | Human Primary CD8+ T | |
|---|---|---|---|
| | | EC₅₀ | Potency |
| No. | Design | (nM) | reduction |
| W3XX115-T2U0.E44-1.uIgG4V322 | / | 7.65 | 1 |
| W3XX115-T2U0.E44-6.uIgG4V322 | Del STLT | 145 | 18.3 |
| W3XX115-T2U0.E44-40.uIgG4V322 | Del STLT, E110R | 282 | 36.9 |
| W3XX115-T2U0.E44-43.uIgG4V322 | Del STLT, K32D | 135 | 17.6 |
| W3XX115-T2U0.E44-44.uIgG4V322 | Del STLT, E52G | 86.2 | 11.3 |
| W3XX115-T2U0.E44-45.uIgG4V322 | Del STLT, K76R | 240 | 31.4 |
| W3XX115-T2U0.E44-46.uIgG4V322 | Del STLT, F78G | 58.4 | 7.6 |
| W3XX115-T2U0.E44-47.uIgG4V322 | Del STLT, P82Y | 211 | 27.6 |

2.2 pSTAT5 Activation Assay of Human Activated CD8+ T Cells

Human CD8+ T cells from fresh PBMC was isolated by EasySep™ Human CD8+ T Cell Isolation Kit (Stemcell-17953). Then human T cell activation/expansion kit (Miltenyi 130-091-441) was used for human CD8+T expansion. Human activated CD8+ T cells (expressing high affinity receptor, IL-2Rα/β/γc) were also analyzed for STAT5 phosphorylation following 30 minutes' incubation with the indicated IL-2 variants. The expanded human activated CD8+ T cells were operated in the same method as described above for CD8+ T cells.

In FIG. 3a and Table 4, monovalent IL-2 variants showed potency reduction in activated CD8+T, T2U0.E44-48 and 49 showed 15-fold and 22-fold potency reduction relative to T2U0.E44-1, just a little stronger than BMK8 in efficiency.

Bivalent IL-2 variants showed extreme potency recovery in activated CD8+T, and potency was comparable or even stronger than WT IL-2 with reduction fold of 0.3-0.9 (Table 4). But the potency of bivalent IL-2 in primary CD8+T was similar to its monovalent variants or even much weaker. Therefore, the potency ratio of IL-2 variants in activated primary CD8+T was sufficiently different in monovalent and bivalent IL-2, bivalent IL-2 variants showed over 10,000 potency ratios of activated CD8+T/primary CD8+T, sharing similar characteristics with BMK8 (o). As a result, bivalent Z20-1, Z20-2, Z20-4 and Z20-5 were biased for IL-2Rα (i.e. CD25) binding, stronger than WT IL-2. The result implied that dimer IL-2 variants were more promising in anti-tumor effect as IL-2Rα-binding is believed to be necessary for IL-2 to exert its biological function.

TABLE 4
Summarized potency of IL-2 variants in activated and primary CD8+ T

| | | Human Activated | | Human Primary | |
|---|---|---|---|---|---|
| | | CD8+ T | | CD8+ T | |
| | | EC₅₀ | Potency | EC₅₀ | Potency |
| No. | Design | (nM) | reduction | (nM) | reduction |
| W3XX115-T2.Z20-1.uIgG4V322 | Del STLT, dimer | 0.005 | 0.3 | 684 | 6.1 |
| W3XX115-T2.Z20-2.uIgG4V322 | Del STLT, I129L, dimer | 0.006 | 0.3 | 401 | 3.5 |
| W3XX115-T2.Z20-4.uIgG4V322 | Del STLT, I129L, E110R, | 0.015 | 0.9 | 511 | 4.5 |
| | dimer | | | | |
| W3XX115-T2.Z20-5.uIgG1V320 | Del STLT, I129L, K32D, | 0.010 | 0.6 | 2383 | 21 |
| | dimer | | | | |
| W3XX115-T2U0.E44-1.uIgG4V322 | WT | 0.017 | 1.0 | 113 | 1.0 |
| W3XX115-BMK8 | L18R, Q22E, Q126K | 0.12 | 7.2 | / | 0.0 |
| W3XX115-T2U0.E44-48.uIgG4V322 | Del STLT, I129L, E110R | 0.25 | 14.7 | 300 | 2.7 |
| W3XX115-T2U0.E44-49.uIgG4V322 | Del STLT, I129L, K32D | 0.38 | 22.2 | 425 | 3.8 |

### Example 3

### In Vitro and In Vivo Stability of IL-2 Variants

### 3.1 Differential Scanning Fluorimetry (DSF)

A DSF assay was performed using 7500 Fast Real-Time PCR system (Applied Biosystems). Briefly, 19 μL of protein solution was mixed with 1 μl of 62.5×SYPRO Orange solution (TheromFisher-S6650) and added to a 96 well plate. The plate was heated from 26° C. to 95° C. at a rate of 2° C./min and the resulting fluorescence data was collected. The data was analyzed automatically by its operation software and Tm was calculated by taking the maximal value of negative derivative of the resulting fluorescence data with respect to temperature. Ton can be roughly determined as the temperature of negative derivative plot beginning to decrease from a pre-transition baseline.

The thermo-stability of IL-2 variants was shown in Table 5, monovalent IL-2 variants showed acceptable Tm1. W3XX115-T2U0.E44-49.uIgG4V322 showed higher Tm than W3XX115-T2U0.E44-26.uIgG4V322, indicating the K32D mutation can stabilize IL-2 molecule and improve its thermal stability. Bivalent IL-2 variants of Z20-1 and Z20-2 showed low Tm1 compared with its monovalent protein. Notably, Z20-5 showed significantly improved thermal stability with Tm1 of 62.6° C. It demonstrates that K32D mutation in Z20-5 could stabilize bivalent IL-2 compared to Z20-2, which was consistent with differential of T2U0.E44-49 and T2U0.E44-26.

TABLE 5
DSF result of IL-2 variants polypeptide complexes

| | | Tm1 |
|---|---|---|
| IL-2 variants | Design | (° C.) |
| W3XX115-T2U0.E44-6.uIgG4V322 | Del STLT, | 60.4 |
| W3XX115-T2U0.E44-26.uIgG4V322 | Del STLT, I129L | 60.6 |
| W3XX115-T2U0.E44-49.uIgG4V322 | Del STLT, I129L, K32D | 63.0 |
| W3XX115-T2.Z20-1.uIgG4V322 | Del STLT, dimer | 56.1 |
| W3XX115-T2.Z20-2.uIgG4V322 | Del STLT, I129L, dimer | 57.2 |
| W3XX115-T2.Z20-5.uIgG4V322 | Del STLT, I129L, K32D, dimer | 62.6 |

### 3.2 Serum Stability Assay

The stability of bivalent IL-2 variants were further determined in mouse serum. In serum stability assay, Z20-1, Z20-4, Z20-5 and BMK8 were diluted in mouse serum in 1:9. After storage at 37 and 4° C. for 4, 7, 10, 14 days, samples were collected and frozen in liquid nitrogen immediately then stored at −80° C. Once all samples were ready, potency determination in activated CD8+T were used to test the samples.

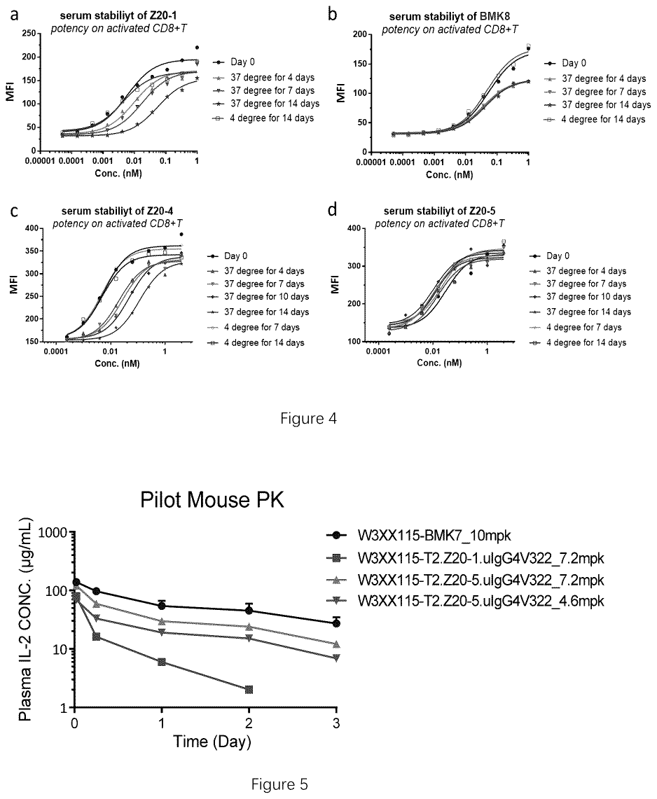

As shown in FIG. 4, after incubation in mouse serum at 37° C., potency of Z20-1 and Z20-4 progressively reduced from 4 to 14 days. Monovalent variant of BMK8 was more stable in mouse serum, with mild efficiency reduction (maximum MFI) at 37° C. By comparison, Z20-5 kept potency and efficiency after incubation for 14 days at 37° C., with its cell activating capability unchanged over time as shown in FIG. 4d. These data demonstrate that K32D mutation in Z20-5 variants stabilizes IL-2 and significantly improves its serum stability. All variants in mouse serum could keep potency at 4° C.

### 3.3 Pharmacokinetics (PK)

To investigate in vivo pharmacokinetics of bivalent IL-2 variants, mouse PK study was conducted. Female C57BL/6 mice (8 to 9 week) from Charles River were randomly assigned to two dosing groups, and injected intravenously with IL-2 variants at different doses. Sample collection for immunology and pharmacokinetics analysis was also performed. Blood samples were collected into tubes without additive and placed on ice until they were processed. All samples were processed within 2 hours of collection.

Drug serum concentrations were measured by ELISA. Briefly, ELISA plate was coated with 1 μg/mL goat anti-human IgG Fc (Southern Biotech, 2049-01), and IL-2 variants concentration in plasma was detected by goat anti-human IgG Fc (Southern Biotech, SB-2049-08) followed by HRP-Streptavidin (Thermo-21127) and TMB substrate (Life Technologies, 002023). The absorbance of the wells was measured at (450-540) nm with a multiwall plate reader (SpectraMax® M5e). Generate standard curve according to the standard samples, and analyze serum samples with SoftMax.

As shown in FIG. 5, K32D mutation in Z20-5 noticeably led to significantly extended serum half-life than Z20-1. Z20-1 showed fast clearance in the distribution and the elimination phase. Z20-5 (7.2 mg/kg) and BMK7 (10 mg/kg) showed similar Co and much slower clearance. Additionally, Z20-5 also showed linear and dose-responsive pharmacokinetics, without causing toxicity and no ADA production. The PK results were consistent with DLS and serum stability data. K32D substitution of bivalent IL-2 variants contributed to extended drug exposure and improved pharmacodynamics.

### 3.4 Modeling of Z20-5

To demonstrate how K32D stabilized Z20-5, full-length sequence of Z20-5 was used for modeling analysis by Discovery Studio and Gromacs, along with reported crystal structure of IL-2.

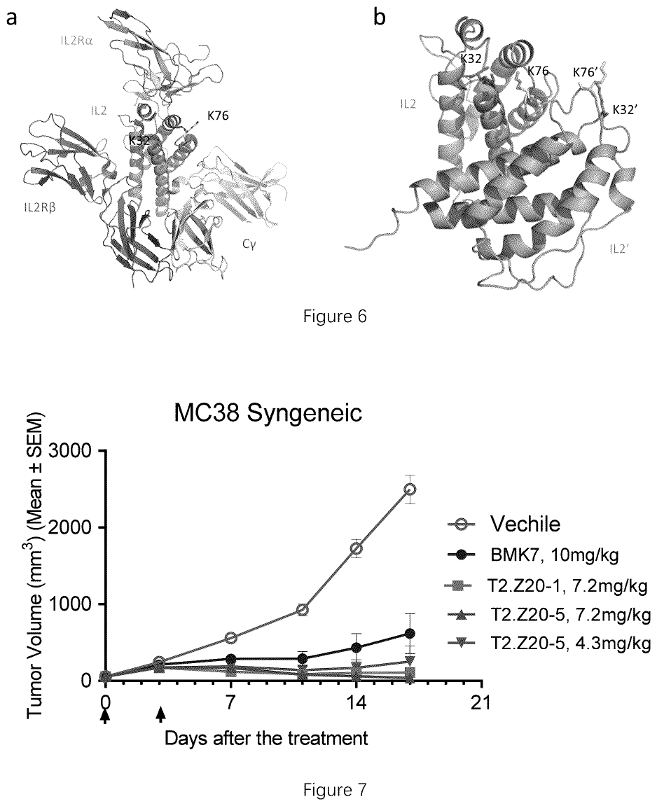

The diagram was shown in FIG. 6a, a K to D substitution at position 32 (K32D) forms a salt-bridge with K76, thus stabilized the IL-2 monomer and resulted in improved stability and pharmacokinetics. The interaction of K32D and K76 also induces minor conformational change in helix A of IL-2, thus affected the binding of IL-2 to the common γ chain, which contributed to further affinity and activity attenuation.

For bivalent IL-2 variants (FIG. 6b), the K32D mutation to the opposite charge can form salt-bridge to the K76 of the same IL-2 moiety. This, along with the resulting conformational change, can reduce the tendency of IL-2 variant aggregation in the Z20 format, thus stabilized bivalent format, which leads to improved serum stability (FIG. 4), prolonged half-life (FIG. 5) and better thermo-stability (Table 5).

### Example 4

### In Vivo Pharmacology of IL-2 Variants

All the procedures related to animal handling, care and the treatment in the study were performed according to the guidelines approved by the Institutional Animal Care and Use Committee (IACUC) of LARC of WuXi Biologics following the guidance of the Association for Assessment and Accreditation of Laboratory Animal Care (AAALAC).

### 4.1 Anti-Tumor Efficacy

In order to explore anti-tumor effect of Z20-5, the stable bivalent IL-2 variant, MC38 syngeneic model were used. The MC38 cells were implanted (s.c.) into the right flanks of C57BL/6N mice. When tumors reach about 60-80 mm³ in volume, tumor bearing mice were randomly divided into different groups, dosing with IL-2 variants at day 0 and day 3. Tumor sizes were measured in two dimensions using a caliper, and the volume was expressed in mm³. Results were represented by mean and the standard error (Mean±SEM). Statistical analysis was operated by two-way ANOVA, P<0.05 was considered as statistically significant.

Firstly, all IL-2 variants retaining CD25-binding (Z20-1, Z20-5, BMK7) showed significant anti-tumor effect in MC38 syngeneic model, as shown in FIG. 7. This demonstrated that IL-2Rα binding is indispensable for IL-2 variants, which relies on stimulating activated CD8+ T to exert anti-tumor effects.

For bivalent IL-2 variants, Z20-1 and Z20-5 at high dose (7.2 mg/kg, equivalent to 10 mg/kg of BMK7) showed better anti-tumor effect than BMK7, with TGIs of 101% and 98% over 77%. Furthermore, Z20-5 in medium dose of 4.3 mg/kg also induced better tumor inhibition than BMK7 in 10 mg/kg. In addition, though the average TGI of Z20-5 was similar to Z20-1 at the same dose, Z20-5 induced 100% complete response (CR) after treatment, while the CR rate of Z20-1 was 60% at day 17. These results demonstrated that the improved stability and prolonged PK led to greater anti-tumor response.

### 4.2 Toxicity of IL-2 Variants

For toxicity study, lungs from 3 mice per group were collected at end of life from the MC38 study in 4.1. Lungs were fixed in formalin, embedded in paraffin and tested with IHC for inflammatory cell infiltration.

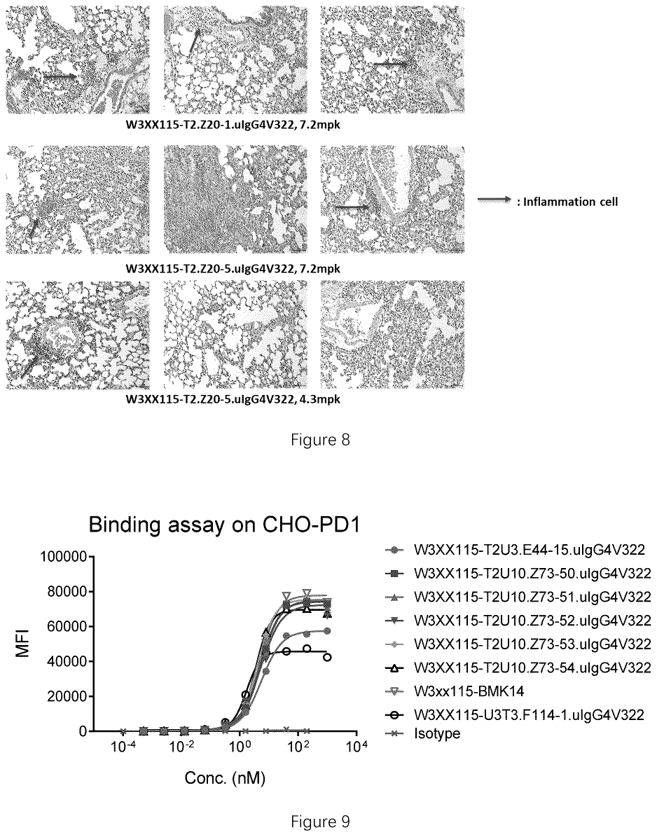

As shown in FIG. 8, there were minimal inflammatory cell infiltration in lungs of all treatment groups. These data showed that Z20-5 has limited toxicity, and the improved stability and prolonged PK does not lead to cumulative toxicity.

Based on PK, efficacy and toxicity results, Z20-5 was a stable bivalent IL-2 variant with prolonged PK and good anti-tumor effect.

### Example 5

### In Vitro and In Vivo Characterization of PD-1/IL-2 Fusion Proteins

### 5.1 PD-1 Binding Assay

CHO-PD1 engineered cell line was used to determine PD-1 binding profile. Cells were incubated with PD-1/IL-2 fusion proteins at the indicated concentrations for 30 min at 4° C. to avoid internalization. Binding was detected by 2^{nd} antibody of PE-anti-human Fc at 4° C. for another 30 min. The median fluorescence intensity (MFI) on CHO-PD1 cells was measured using FACS.

As shown in FIG. 9, PD-1/IL-2 fusion proteins in Z73 format showed comparable PD-1 binding profile with W3XX115-BMK14. W3XX115-U3T3.F114-1.uIgG4V322 and W3XX115-T2U3.E44-15.uIgG4V322 also showed similar affinity to PD-1/IL-2 fusion proteins in Z73 format, although the maximum binding MFI were a little lower which might be dominated by different PD-1 sequences.

5.2 pSTAT5 Activation Assay of PD-1/IL-2 Fusion Proteins

STAT5 analysis were described as above, the phosphorylation level of STAT5 in the primary and activated CD8+T populations was determined. Activated CD8+ T cells were isolated from fresh PBMC and activated by human T cell activation/expansion kit (Miltenyi 130-091-441) for 5-7 days. The activated CD8+ T cells were CD25 positive and PD-1 positive.

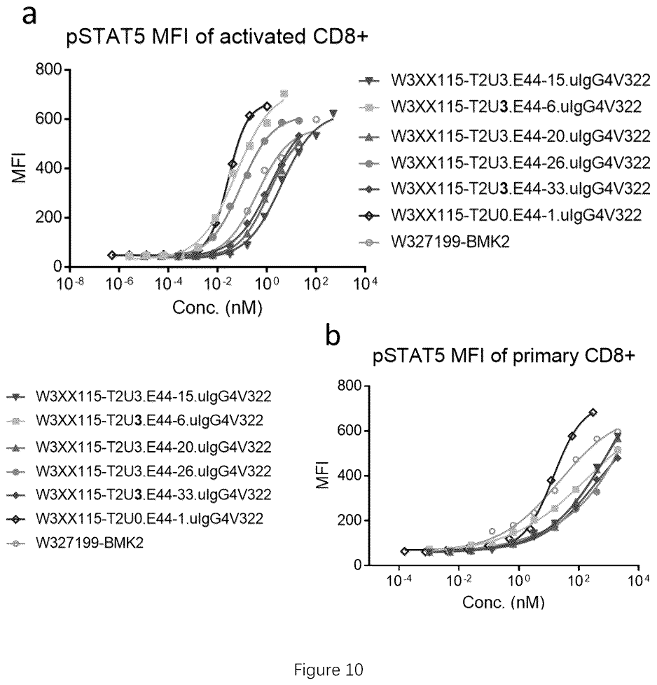

The results were shown in FIG. 10 and Table 6, PD-1/IL-2 fusion proteins in E44 format showed potency reduction in different degree on primary CD8+ T cells (PD-1−) which was generated by IL-2Rβγ attenuation. On activated CD8+ T cells (PD-1+), all PD-1/IL-2 fusion proteins were more potent than that on primary CD8+ T cells (PD-1−). T2U3.E44-6 and 26 (preserved IL-2Rα binding) showed strong potency on activated CD8+ T cells versus primary cells, with 3473-fold and 4205-fold PD-1+/PD-1 ratio, which was dominated by both IL-2Rα and PD-1 binding. Besides, T2U3.E44-20 with weak IL-2Rα binding also showed bias on PD-1+activated CD8+ T cells, and the ratio was 669. For IL-2 variants without IL-2Rα binding, T2U3.E44-15 and 33 showed PD-1+/PD-1-ratio of 496 and 283, the bias was dominated by PD-1 alone.

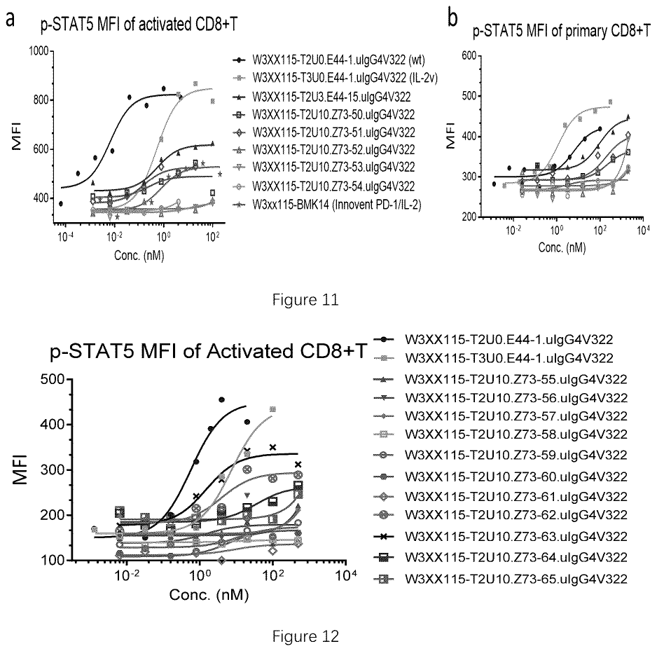

Besides, IL-2 variants of PD-1/IL-2 in Z73 format was designed as abolished IL-2Rα binding, that means activated CD8+ T cell-bias was dominated by PD-1 anchor. T2U10.Z73-50/51/52/53/54 showed attenuated potency on primary and activated CD8+ T cells in varying degrees (FIG. 11 and Table 7). And PD-1 fusion derived better potency on activated CD8+ T cells.

More IL-2 variants were designed and fused with PD-1 antibody. T2U10.Z73-55 to T2U10.Z73-64 showed further attenuation on IL-2Rβγ as shown in FIG. 12 and Table 8.

TABLE 6
Summarized potency of PD-1/IL-2 variants in E44 format on activated and primary CD8+T

| | | Human Primary | | Human Activated | | |
|---|---|---|---|---|---|---|
| | | CD8+ T | | CD8+ T | | |
| | | | Potency | | Potency | PD-1+/ |
| PD-1/IL-2 fusion protein | IL-2 Design | EC₅₀ (nM) | reduction | EC₅₀ (nM) | reduction | PD-1- |
| W3XX115-T2U0.E44-1.uIgG4V322 | WT | 14.24 | 1.0 | 0.027 | 1.0 | 527 |
| W327199-BMK2 | Weak IL-15 | 22.73 | 1.6 | 0.41 | 15.2 | 55 |
| W3XX115-T2U3.E44-6.uIgG4V322 | βγ attenuation | 191 | 13.4 | 0.055 | 2.0 | 3473 |
| W3XX115-T2U3.E44-26.uIgG4V322 | βγ attenuation | 370 | 25.9 | 0.088 | 3.2 | 4205 |
| W3XX115-T2U3.E44-15.uIgG4V322 | Non-α, βγ attenuation | 1873 | 132 | 3.78 | 139 | 496 |
| W3XX115-T2U3.E44-20.uIgG4V322 | weak α, βγ attenuation | 809 | 56.8 | 1.21 | 44.3 | 669 |
| W3XX115-T2U3.E44-33.uIgG4V322 | Non-α, βγ attenuation | 373 | 26.2 | 1.32 | 48.6 | 283 |

TABLE 7
Summarized potency of PD-1/IL-2 variants in Z73 format on activated and primary CD8+ T

| | | Human Activated | | Human Primary | |
|---|---|---|---|---|---|
| | | CD8+ T | | CD8+ T | |
| | | EC₅₀ | Potency | EC₅₀ | Potency |
| PD-1/IL-2 fusion protein | IL-2 Design | (nM) | reduction | (nM) | reduction |
| W3XX115-T2U0.E44-1.uIgG4V322 | WT | 6.0 | 1.0 | 0.007 | 1.0 |
| W3XX115-T3U0.E44-1.uIgG4V322 | F42V/Y45A/L72G | 1.14 | 0.2 | 0.521 | 79 |
| W3xx115-BMK14 | N88D | NA | / | 0.788 | 120 |
| W3XX115-T2U3.E44-15.uIgG4V322 | del C terminal STLT, F42I | 92 | 15 | 0.534 | 81 |
| W3XX115-T2U10.Z73-50.uIgG4V322 | del C terminal STLT, | 213 | 36 | 0.246 | 38 |
| | F42I/E110R/I129L | | | | |
| W3XX115-T2U10.Z73-51.uIgG4V322 | del C terminal STLT, | 185 | 31 | 0.153 | 23 |
| | K32D/F42I/I129L | | | | |
| W3XX115-T2U10.Z73-52.uIgG4V322 | L18R/Q22E/F42I/Q126K | NA | / | NA | / |
| W3XX115-T2U10.Z73-53.uIgG4V322 | L19H/F42I/C125I Q126E | NA | / | NA | / |
| W3XX115-T2U10.Z73-54.uIgG4V322 | T3A//D20N/F42I/N71K/C125S | NA | / | NA | / |

TABLE 8
Summarized potency of PD-1/non-α IL-2 variants in
activated and primary CD8+ T

| | Human Activated |
|---|---|
| | CD8+ T |
| PD-1/IL-2 fusion protein | Potency reduction |
| W3XX115-T2U0.E44-1.uIgG4V322 | 1.0 |
| W3XX115-T3U0.E44-1.uIgG4V322 | 12.4 |
| W3XX115-T2U10.Z73-55.uIgG4V322 | NA |
| W3XX115-T2U10.Z73-56.uIgG4V322 | 2.5 |
| W3XX115-T2U10.Z73-57.uIgG4V322 | NA |
| W3XX115-T2U10.Z73-58.uIgG4V322 | 26.1 |
| W3XX115-T2U10.Z73-59.uIgG4V322 | 18.9 |
| W3XX115-T2U10.Z73-60.uIgG4V322 | 10.0 |
| W3XX115-T2U10.Z73-61.uIgG4V322 | 14.2 |
| W3XX115-T2U10.Z73-62.uIgG4V322 | 6.5 |
| W3XX115-T2U10.Z73-63.uIgG4V322 | 2.5 |
| W3XX115-T2U10.Z73-64.uIgG4V322 | 58.7 |
| W3XX115-T2U10.Z73-65.uIgG4V322 | NA |

### 5.3 Anti-Tumor Efficacy

In order to explore anti-tumor effect of PD-1/IL-2 fusion proteins, CT-26 syngeneic model were used. The CT-26 cells were implanted (s.c.) into the right flanks of Balb.c mice. When tumors reach about 60-80 mm³ in volume, tumor bearing mice were randomly divided into different groups, dosing with PD-1/IL-2 fusion proteins at day 0 and day 3. Tumor sizes were measured in two dimensions using a caliper, and the volume was expressed in mm³. Results were represented by mean and the standard error (Mean±SEM). Statistical analysis was operated by two-way ANOVA, P<0.05 was considered to be statistically significant.

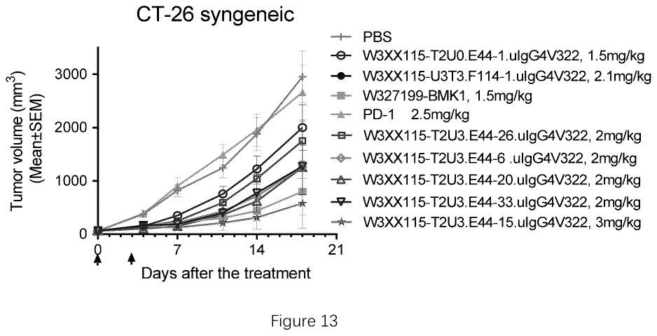

CT-26 was a PD-1 resistant model as shown in FIG. 13, and exhibited partial response to IL-2 (W3XX115-T2U0.E44-1.uIgG4V322). PD-1 fusion IL-2 or IL-15 showed enhanced tumor suppression than IL-2 alone, where T2U3.E44-6/20/33 held similar effect and better than T2U3.E44-26. T2U3.E44-15 showed the most significant anti-tumor effect, even slightly better than W327199-BMK1. As a result, PD-1 binding moiety fused with non-α IL-2 generated efficient tumor suppression.

Those skilled in the art will further appreciate that the present invention may be embodied in other specific forms without departing from the spirit or central attributes thereof. In that the foregoing description of the present disclosure provides only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1.-44. (canceled)

45. A composition comprising a polypeptide complex or a nucleic acid molecule(s) encoding the polypeptide complex as active ingredient, and an excipient,
wherein the polypeptide complex comprises an interleukin-2 (IL-2) variant domain, a first dimerization domain and a second dimerization domain,
wherein the IL-2 variant domain comprises a truncation of 4 amino acids from the C terminal with reference to SEQ ID NO: 1, and
wherein the first dimerization domain and the second dimerization domain associate together to form a dimer.

46. The composition of claim 45, wherein the polypeptide complex or the nucleic acid molecule encoding the polypeptide complex is less than 90%, less than 80%, less than 70%, less than 60% or less than 50% by weight of the composition.

47. The composition of claim 45, wherein the IL-2 variant domain comprises a K32D substitution as compared to SEQ ID NO: 1.

48. The composition of claim 47, wherein the IL-2 variant domain further comprises one of the following groups of substitutions:
(a) an I129L substitution as compared to SEQ ID NO: 1;
(b) an F42I substitution as compared to SEQ ID NO: 1;
(c) I129L and F42I substitutions, optionally further comprising a substitution selected from Q13D, D84K, S87R, N88K, V91E, I92R, I92D, L94D, E95R, N119R, T123K, Q13R, D84T, S87I, V91S, E95Y, N119Q, and T123V as compared to SEQ ID NO: 1.

49. The composition of claim 45, wherein the IL-2 variant domain comprises or consists of the amino acid sequence as shown in any of SEQ ID Nos: 13, 12, 2-3, 79-82, 86-103, 4-11 and 111.

50. The composition of claim 45, wherein the polypeptide complex comprises:
(a) two IL-2 variant domains in two chains respectively, each chain comprises from N terminal to C terminal one IL-2 variant domain operably linked to one dimerization domain;
(b) one IL-2 variant domain and one antigen-binding moiety in Fab format, the polypeptide complex comprises two heavy chains and one light chain, wherein from N-terminal to C-terminal:
the first heavy chain comprises the IL-2 variant domain operably linked to the first dimerization domain,
the second heavy chain comprises the heavy chain of the Fab operably linked to the second dimerization domain, and
the light chain comprises the light chain of the Fab;
(c) one IL-2 variant domain and two antigen-binding moieties in VHH format, the polypeptide complex comprises two chains, wherein from N-terminal to C-terminal:
the first chain comprises the IL-2 variant domain operably linked to the first dimerization domain, and
the second chain comprises two VHHs in tandem operably linked to the second dimerization domain;
(d) one IL-2 variant domain and one antigen-binding moiety in VHH format, the polypeptide complex comprises two chains, wherein from N-terminal to C-terminal:
the first chain comprises the IL-2 variant domain operably linked to the first dimerization domain, and
the second chain comprises the VHH operably linked to the second dimerization domain;
(e) two IL-2 variant domains and two antigen-binding moieties in Fab format, the polypeptide complex comprises two heavy chains and two light chains, wherein from N-terminal to C-terminal:
each heavy chain comprises the IL-2 variant domain operably linked to the first or second dimerization domain, which is operably linked to the heavy chain of the Fab, and
each light chain comprises the light chain of the Fab; or
(f) two IL-2 variant domains and two antigen-binding moieties in VHH or scFv format, the polypeptide complex comprises two chains, wherein from N-terminal to C-terminal:
each chain comprises the IL-2 variant domain operably linked to the first or second dimerization domain, which is operably linked to the VHH or scFv.

51. The composition of claim 50, wherein the antigen-binding moiety specifically binds to an antigen selected from a Tumor associated antigen (TAA), an I/O checkpoint, a tumor microenvironment target, an autoimmune associated target and an inflammatory disease associated target.

52. The composition of claim 45, wherein the first dimerization domain is one chain of an immunoglobulin Fc region, and the second dimerization domain is the other chain of the immunoglobulin Fc region, wherein the Fc region is a IgG4, IgG1, IgG2 or IgG3 Fc region, and optionally comprises one or more substitutions compared to wild type human Fc to promote heterodimerization or homodimerization, to extend half-life, to alter effector functions or to remove N-glycosylation.

53. The composition of claim 45, wherein the polypeptide complex comprises:
(a) an amino acid sequence of any of SEQ ID NOs: 32, 29, 30 and 31;
(b) a first heavy chain comprising the amino acid sequence of any of SEQ ID NOs: 52-56, a second heavy chain comprising the amino acid sequence of SEQ ID NO: 57, and a light chain comprising the amino acid sequence of SEQ ID NO: 58;
(c) a first chain comprising the amino acid sequence of any of SEQ ID NOs: 59-74, 76-78 and 104-110, and a second chain comprising the amino acid sequence of SEQ ID NO: 75; or
(d) the first heavy chain comprises the amino acid sequence of any of SEQ ID Nos: 17-28, the second heavy chain comprises the amino acid sequence of SEQ ID NO: 33, and the light chain comprises the amino acid sequence of SEQ ID NO: 34.

54. An interleukin-2 (IL-2) variant, wherein the IL-2 variant comprises the amino acid sequence of the IL-2 variant domain of claim 45.

55. A polypeptide complex, wherein the polypeptide complex comprises an interleukin-2 (IL-2) variant domain, a first dimerization domain and a second dimerization domain,
wherein the IL-2 variant domain comprises the IL-2 variant of claim 54, and
wherein the first dimerization domain and the second dimerization domain associate together to form a dimer.

56. The polypeptide complex of claim 55, comprising one IL-2 variant domain and two antigen-binding moieties in VHH format, wherein the polypeptide complex comprises two chains, from N-terminal to C-terminal:
the first chain comprises the IL-2 variant domain operably linked to the first dimerization domain; and
the second chain comprises two VHHs in tandem operably linked to the second dimerization domain.

57. The polypeptide complex of claim 56, wherein:
the first chain comprises the amino acid sequence of any of SEQ ID Nos: 59-74, 76-78 and 104-110;
the second chain comprises the amino acid sequence of SEQ ID NO: 75.

58. An isolated nucleic acid molecule, comprising a nucleic acid sequence(s) encoding the IL-2 variant of claim 54.

59. A vector or host cell comprising the nucleic acid molecule of claim 58.

60. A method for treating or preventing a cancer, an autoimmune or an inflammatory disease or modulating an immune response which is NK cell, CD8+ cell, or CD4+ T cell (especially Treg) related in a subject, comprising administering an effective amount of the composition of claim 45 to the subject.

61. The method of claim 60, which further comprises administering an additional anti-tumor therapy, such as cell immunotherapy including tumor-infiltrating lymphocyte (TIL) therapy, T cell receptor (TCR) therapy, chimeric antigen receptor (CAR) T cell therapy, macrophage cell therapy, and NK cell therapy, targeted therapy, chemotherapy and gene therapy (e.g. a gene therapy that uses lentivirus, AAV, poxvirus, herpes zoster virus, oncolytic virus, or other RNA/DNA vectors).

62. The method of claim 60, wherein the cancer is selected from colon cancer, breast cancer, lung cancer (such as NSCLC), ovarian cancer, melanoma, bladder cancer, renal cell carcinoma, liver cancer, prostate cancer, stomach cancer, pancreatic cancer, lymphoma (such as non-Hodgkin's lymphoma and diffuse large B-cell lymphoma), leukemia (such as chronic lymphocytic leukemia) and multiple myeloma, optionally the cancer is a PD-1 related cancer.

63. The method of claim 60, wherein the autoimmune or inflammatory disease is selected from inflammatory bowel disease, multiple sclerosis, rheumatoid arthritis, systemic lupus erythematosus, aplastic anemia, coeliac disease, type1 diabetes, graves' disease, psoriasis, scleroderma.

64. A kit comprising a container comprising the composition of claim 45.
